(19) United States
(12) Patent Application Publication
(10) Pub. No.: US 2017/0354175 A1
(43) Pub. Date: Dec. 14, 2017

(54) SWEETENER COMPOSITION

(71) Applicant: Senomyx, Inc., San Diego, CA (US)

(72) Inventors: Donald S. Karanewsky, Escondido, CA (US); Catherine Tachdjian, San Diego, CA (US); Guy Servant, San Diego, CA (US); Tanya Ditschun, San Diego, CA (US)

(21) Appl. No.: 15/595,428

(22) Filed: May 15, 2017

(51) Int. Cl. A23L 27/30; A23L 27/20; C07D 285/22

(52) U.S. Cl. CPC A23L 27/30; A23L 27/205; C07D 285/22

(57) ABSTRACT

Formulations comprising one or more sweetener and one or more flavor modifying compound are provided herein, wherein the formulation is provided for use in ingestible products, such as food or beverage products or pharmaceutical, or for use in non-comestible products, such as cosmetic or hygienic products.

### BACKGROUND

### Field

The present disclosure relates to the fields of chemistry and flavor modifying compounds and sweeteners in foods, beverages, animal feed, pharmaceuticals, and other ingestible compositions. More specifically, the present disclosure relates to product combinations that include a sweet tasting compound and a flavor modifying compound.

### Background Description

The taste system provides sensory information about the chemical composition of the external world. Taste transduction is one of the most sophisticated forms of chemical-triggered sensation in animals. Signaling of taste is found throughout the animal kingdom, from simple metazoans to the most complex of vertebrates. Mammals are believed to recognize five basic taste modalities: sweet, bitter, sour, salty, and umami (the taste of monosodium glutamate, or savory taste), through specific chemosensory receptors and ion channels located at the surface of taste receptor cells located in taste buds.

Obesity, diabetes, and cardiovascular disease are health concerns on the rise globally, but are growing at alarming rates in the United States. Sugar and calories are key components that can be limited to render a positive nutritional effect on health. High-intensity sweeteners can provide the sweetness of sugar, with various taste qualities. Because they are many times sweeter than sugar, much less of the sweetener is required to replace the sugar. As a result, many industries are using sweeteners in place of sugar.

However, sweeteners such as saccharin and 6-methyl-1,2,3-oxathiazin-4(3H)-one-2,2-dioxide potassium salt (acesulfame potassium) are commonly characterized as having undesirable bitter and/or metallic aftertastes. Products prepared with 2,4-dihydroxybenzoic acid are claimed to display reduced undesirable aftertastes associated with sweeteners, and do so at concentrations below those concentrations at which their own tastes are perceptible. In contrast, some high-intensity sweeteners, notably sucralose (1,6-dichloro-1,6-dideoxy-β-D-fructofuranosyl-4-chloro-4-deoxy-α-D-galacto-pyranoside) and aspartame (N-L-α-aspartyl-L-phenylalanine methyl ester), display clean sweet tastes very similar to that of sugar (S. G. Wiet and G. A. Miller, Food Chemistry, 58(4):305-311 (1997)). In other words, these compounds are not characterized as having bitter or metallic aftertastes.

Some high intensity sweeteners such as sucralose and aspartame are reported to have sweetness delivery problems, for example, delayed onset and lingering of sweetness (S. G. Wiet, et al., J. Food Sci., 58(3):599-602, 666 (1993)).

Hence, there is a need for new sweetening compounds, sweet taste enhancers and modifiers, and compositions containing such compounds, enhancers, and modifiers with improved taste.

### SUMMARY

Some embodiments provided herein relate to a formulation including one or more sweetener in combination with one or more flavor modifying compound.

In some embodiments, the sweetener is a compound with sweet flavor including without any limitation any natural or synthesized sweet flavor compound that elicits a perception of sweetness. In some embodiments, the sweetener is a non-caloric sweetener, a reduced calorie sweetener, or a non-target caloric sweetener.

In some embodiments, the flavor modifying compound is a flavor modifying compound as disclosed in U.S. Application Publication No. 2008/0306093, entitled “Modulation of Chemosensory Receptors and Ligands Associated Therewith”, filed Jun. 8, 2007, which is incorporated herein by reference in its entirety. In some embodiments, the flavor modifying compound is an embodiment of a flavor modifying compound as disclosed in U.S. Application Publication No. 2014/0094453, entitled “Sweet flavor modifier”, filed Dec. 4, 2014, which is incorporated herein by reference in its entirety. In some embodiments, the flavor modifying compound is an embodiment of a flavor modifying compound as disclosed in U.S. Application Publication No. 2014/0235624, entitled “Sweet flavor modifier”, filed Feb. 19, 2014, which is incorporated herein by reference in its entirety. In some embodiments, the flavor modifying compound is an embodiment of a flavor modifying compound as disclosed in U.S. Application Publication No. 2015/0376176, entitled “Sweet flavor modifier”, filed Aug. 14, 2015, which is incorporated herein by reference in its entirety.

Some embodiments provided herein relate to a formulation having a sweetener and one or more flavor modifying compound.

In some embodiments, the flavor modifying compound is a compound having a cyclic thiadiazine core. In some embodiments, the flavor modifying compound is defined by the compound of:

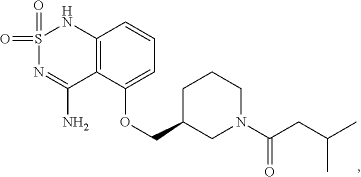 [SMILES: CC(C)CC(=O)N1CCC[C@H](COc2cccc3c2C(N)=NS(=O)(=O)N3)C1]

or a salt or solvate thereof.

In some embodiments, the flavor modifying compound is defined by the compound of:

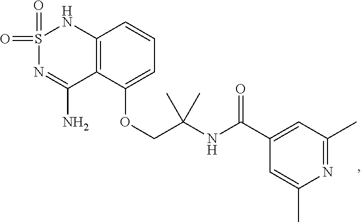 [SMILES: Cc1cc(C(=O)NC(C)(C)COc2cccc3c2C(N)=NS(=O)(=O)N3)cc(C)n1]

or a salt or solvate thereof.

In some embodiments, a flavor modifying compound is a compound selected from the group consisting of:

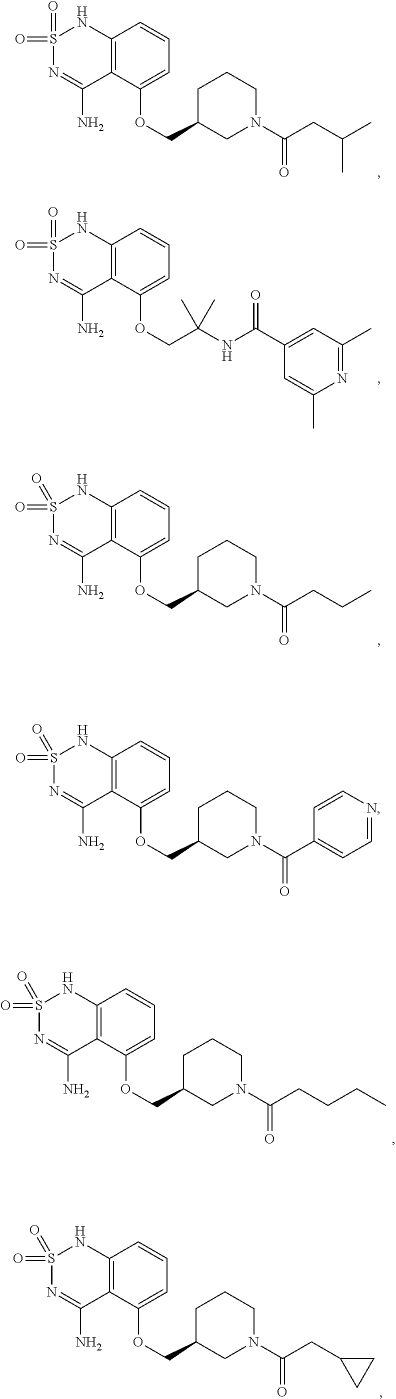

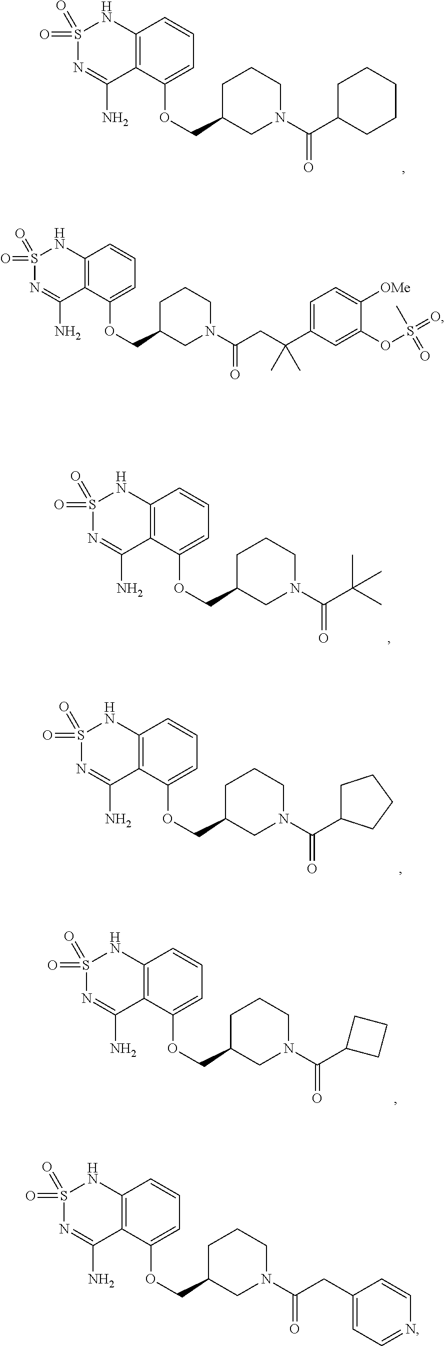

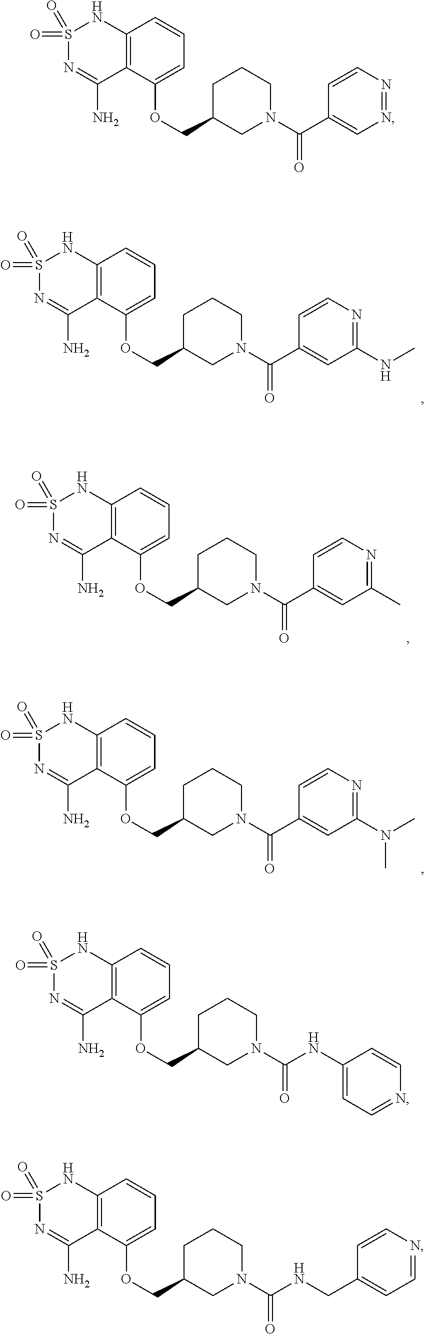

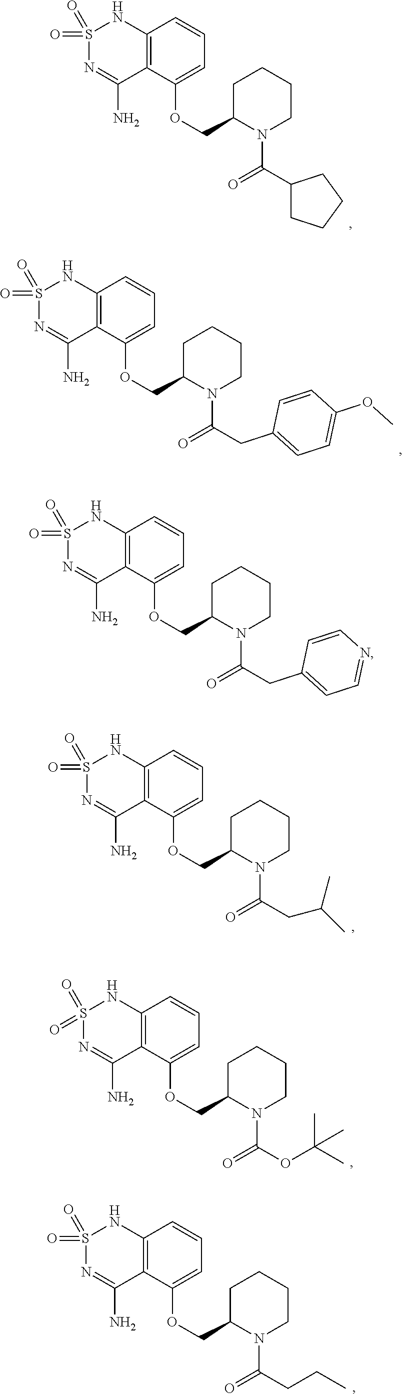

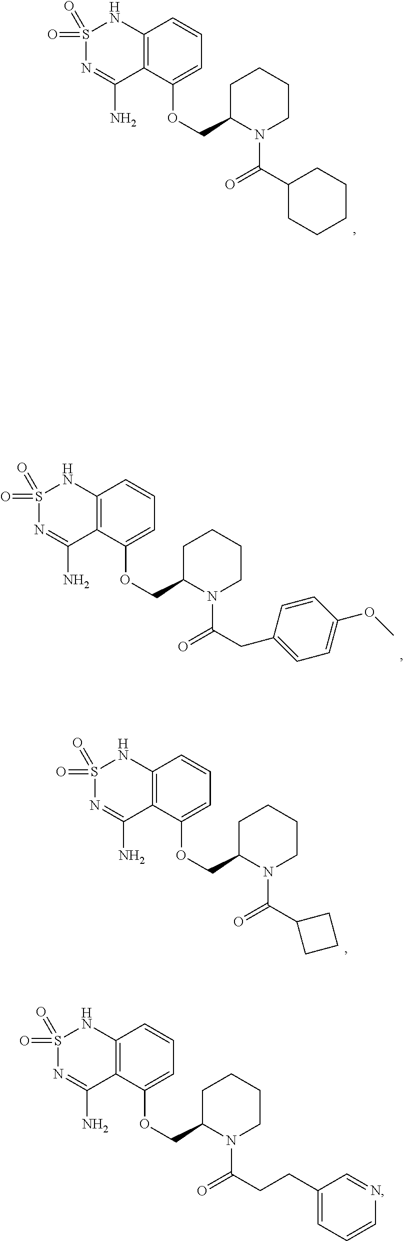

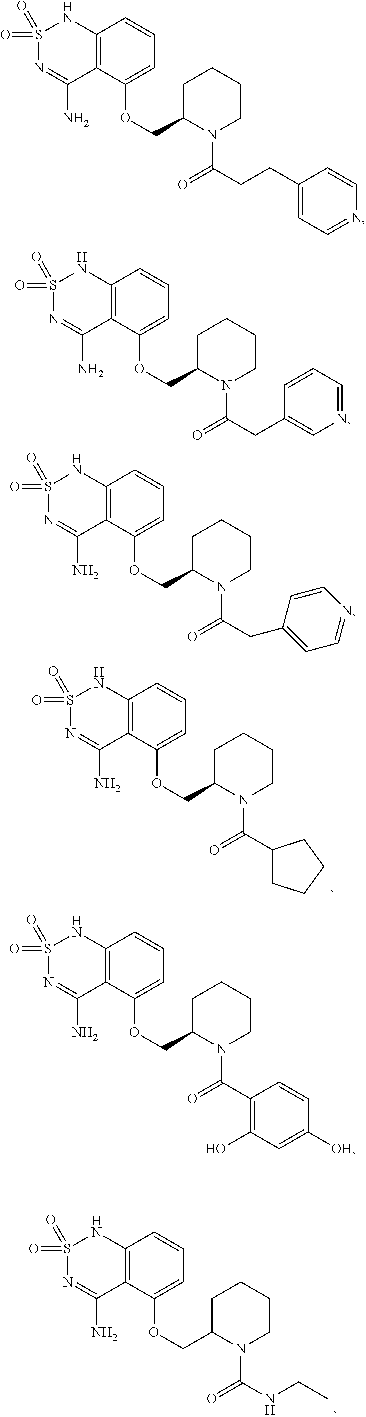 [SMILES: NC1=NS(=O)(=O)Nc2cccc(OC[C@H]3CCCCN3C(=O)CCc3ccncc3)c21 ; NC1=NS(=O)(=O)Nc2cccc(OC[C@H]3CCCCN3C(=O)Cc3cccnc3)c21 ; NC1=NS(=O)(=O)Nc2cccc(OC[C@H]3CCCCN3C(=O)Cc3ccncc3)c21 ; NC1=NS(=O)(=O)Nc2cccc(OC[C@H]3CCCCN3C(=O)C3CCCC3)c21 ; NC1=NS(=O)(=O)Nc2cccc(OC[C@H]3CCCCN3C(=O)c3ccc(O)cc3O)c21 ; CCNC(=O)N1CCCCC1COc1cccc2c1C(N)=NS(=O)(=O)N2]

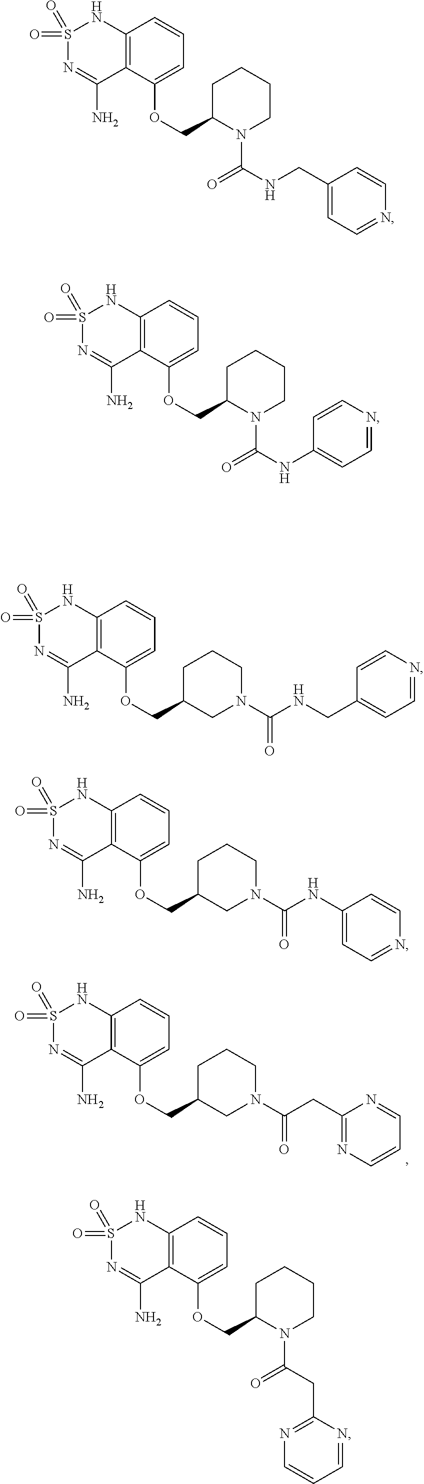 [SMILES: NC1=NS(=O)(=O)Nc2cccc(OC[C@H]3CCCCN3C(=O)NCc3ccncc3)c21 ; NC1=NS(=O)(=O)Nc2cccc(OC[C@H]3CCCCN3C(=O)Nc3ccncc3)c21 ; NC1=NS(=O)(=O)Nc2cccc(OC[C@H]3CCCN(C(=O)NCc4ccncc4)C3)c21 ; NC1=NS(=O)(=O)Nc2cccc(OC[C@H]3CCCN(C(=O)Nc4ccncc4)C3)c21 ; NC1=NS(=O)(=O)Nc2cccc(OC[C@H]3CCCN(C(=O)Cc4ncccn4)C3)c21 ; NC1=NS(=O)(=O)Nc2cccc(OC[C@H]3CCCCN3C(=O)Cc3ncccn3)c21]

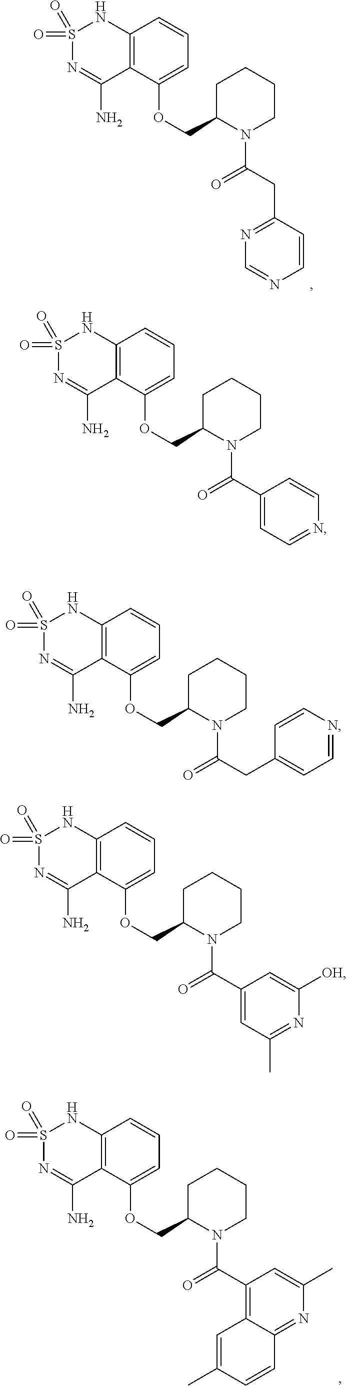 [SMILES: NC1=NS(=O)(=O)Nc2cccc(OC[C@H]3CCCCN3C(=O)Cc3ccncn3)c21 ; "," ; NC1=NS(=O)(=O)Nc2cccc(OC[C@H]3CCCCN3C(=O)c3ccncc3)c21 ; NC1=NS(=O)(=O)Nc2cccc(OC[C@H]3CCCCN3C(=O)Cc3ccncc3)c21 ; Cc1cc(C(=O)N2CCCC[C@@H]2COc2cccc3c2C(N)=NS(=O)(=O)N3)cc(O)n1 ; Cc1ccc2nc(C)cc(C(=O)N3CCCC[C@@H]3COc3cccc4c3C(N)=NS(=O)(=O)N4)c2c1]

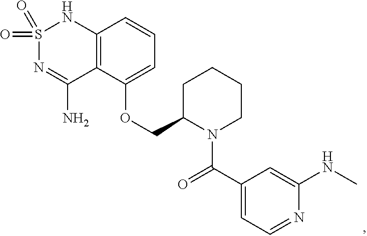 [SMILES: CNc1cc(C(=O)N2CCCC[C@@H]2COc2cccc3c2C(N)=NS(=O)(=O)N3)ccn1]

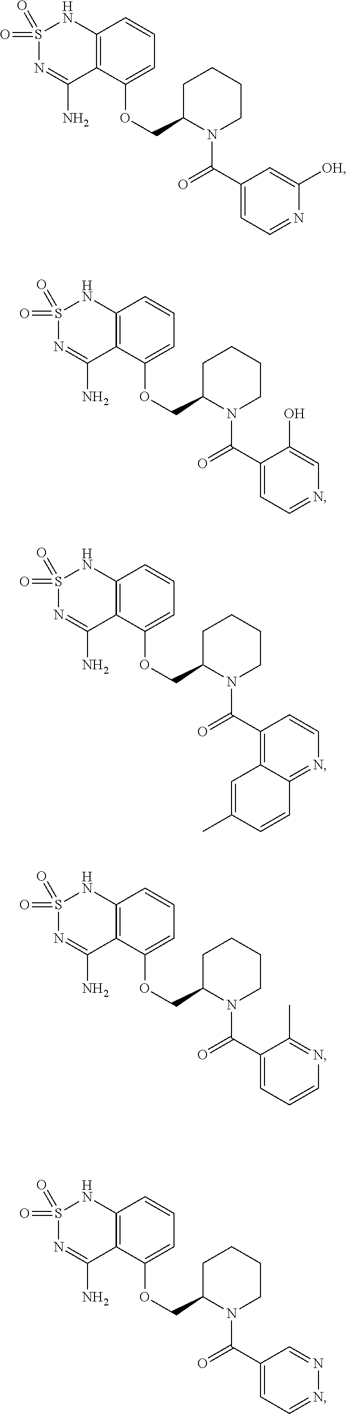 [SMILES: NC1=NS(=O)(=O)Nc2cccc(OC[C@H]3CCCCN3C(=O)c3ccnc(O)c3)c21 ; NC1=NS(=O)(=O)Nc2cccc(OC[C@H]3CCCCN3C(=O)c3ccncc3O)c21 ; Cc1ccc2nccc(C(=O)N3CCCC[C@@H]3COc3cccc4c3C(N)=NS(=O)(=O)N4)c2c1 ; Cc1ncccc1C(=O)N1CCCC[C@@H]1COc1cccc2c1C(N)=NS(=O)(=O)N2 ; NC1=NS(=O)(=O)Nc2cccc(OC[C@H]3CCCCN3C(=O)c3ccnnc3)c21]

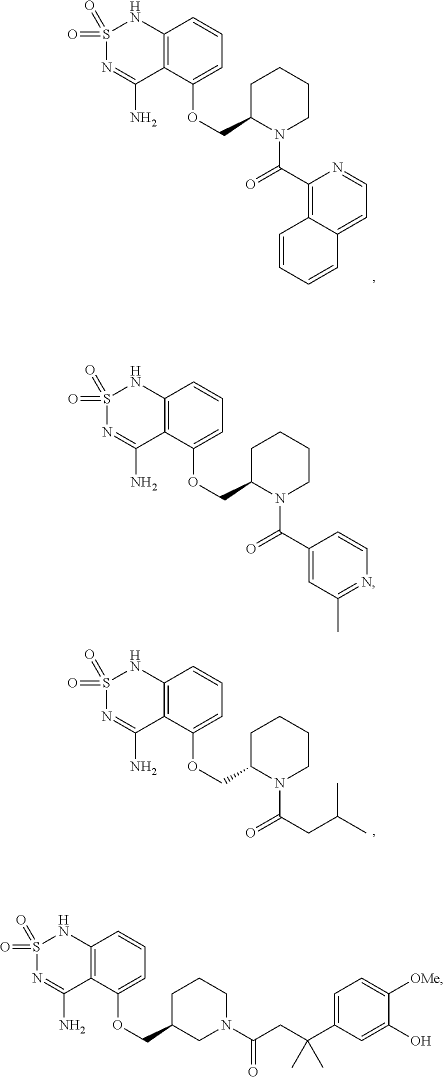 [SMILES: NC1=NS(=O)(=O)Nc2cccc(OC[C@H]3CCCCN3C(=O)c3nccc4ccccc34)c21 ; Cc1cc(C(=O)N2CCCC[C@@H]2COc2cccc3c2C(N)=NS(=O)(=O)N3)ccn1 ; CC(C)CC(=O)N1CCCC[C@H]1COc1cccc2c1C(N)=NS(=O)(=O)N2 ; COc1ccc(C(C)(C)CC(=O)N2CCC[C@H](COc3cccc4c3C(N)=NS(=O)(=O)N4)C2)cc1O]

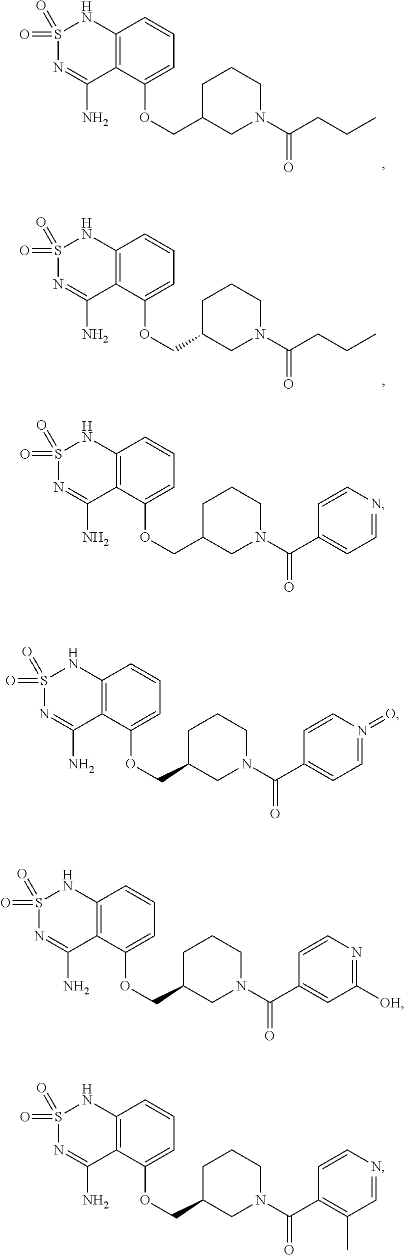 [SMILES: CCCC(=O)N1CCCC(COc2cccc3c2C(N)=NS(=O)(=O)N3)C1 ; CCCC(=O)N1CCC[C@@H](COc2cccc3c2C(N)=NS(=O)(=O)N3)C1 ; NC1=NS(=O)(=O)Nc2cccc(OCC3CCCN(C(=O)c4ccncc4)C3)c21 ; NC1=NS(=O)(=O)Nc2cccc(OC[C@H]3CCCN(C(=O)c4cc[n+]([O-])cc4)C3)c21 ; NC1=NS(=O)(=O)Nc2cccc(OC[C@H]3CCCN(C(=O)c4ccnc(O)c4)C3)c21 ; Cc1cnccc1C(=O)N1CCC[C@H](COc2cccc3c2C(N)=NS(=O)(=O)N3)C1]

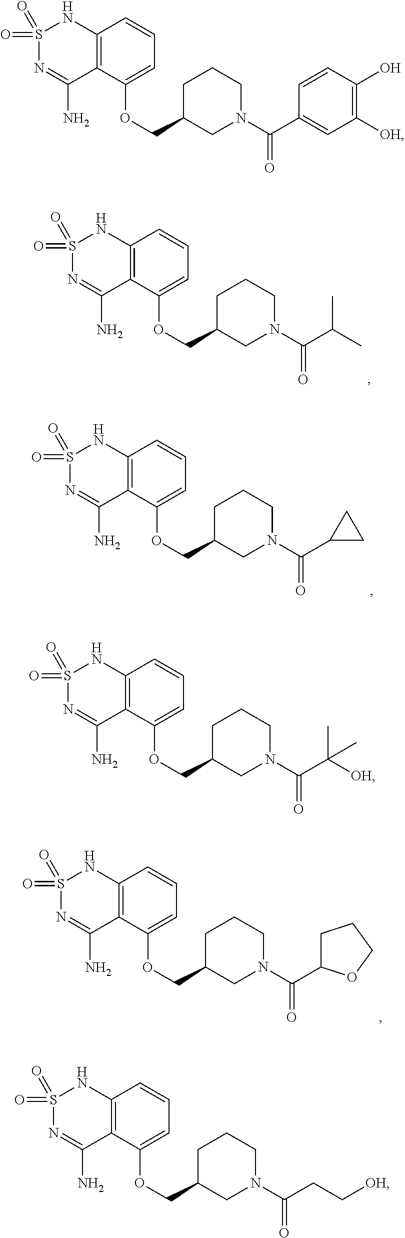

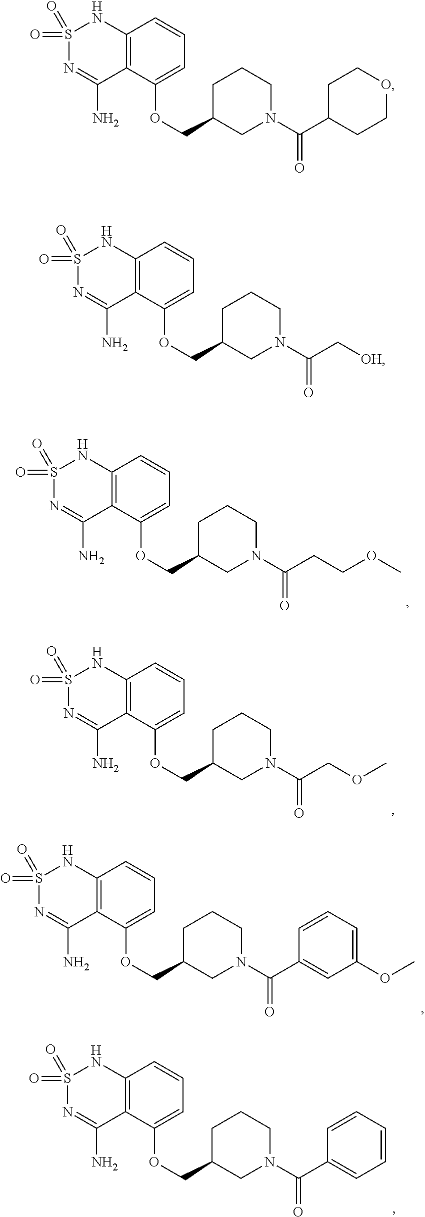 [SMILES: NC1=NS(=O)(=O)Nc2cccc(OC[C@H]3CCCN(C(=O)C4CCOCC4)C3)c21 ; NC1=NS(=O)(=O)Nc2cccc(OC[C@H]3CCCN(C(=O)CO)C3)c21 ; COCCC(=O)N1CCC[C@H](COc2cccc3c2C(N)=NS(=O)(=O)N3)C1 ; COCC(=O)N1CCC[C@H](COc2cccc3c2C(N)=NS(=O)(=O)N3)C1 ; COc1cccc(C(=O)N2CCC[C@H](COc3cccc4c3C(N)=NS(=O)(=O)N4)C2)c1 ; NC1=NS(=O)(=O)Nc2cccc(OC[C@H]3CCCN(C(=O)c4ccccc4)C3)c21]

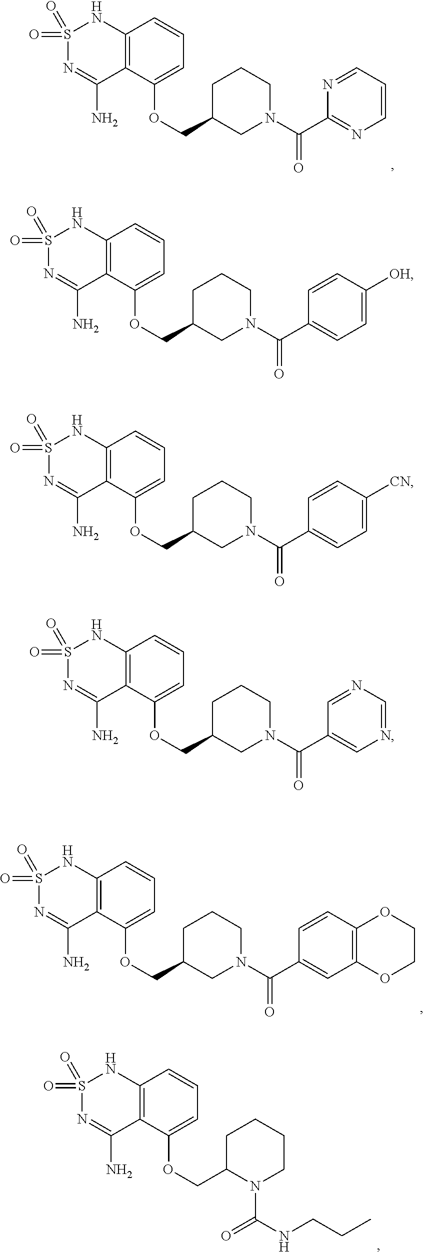

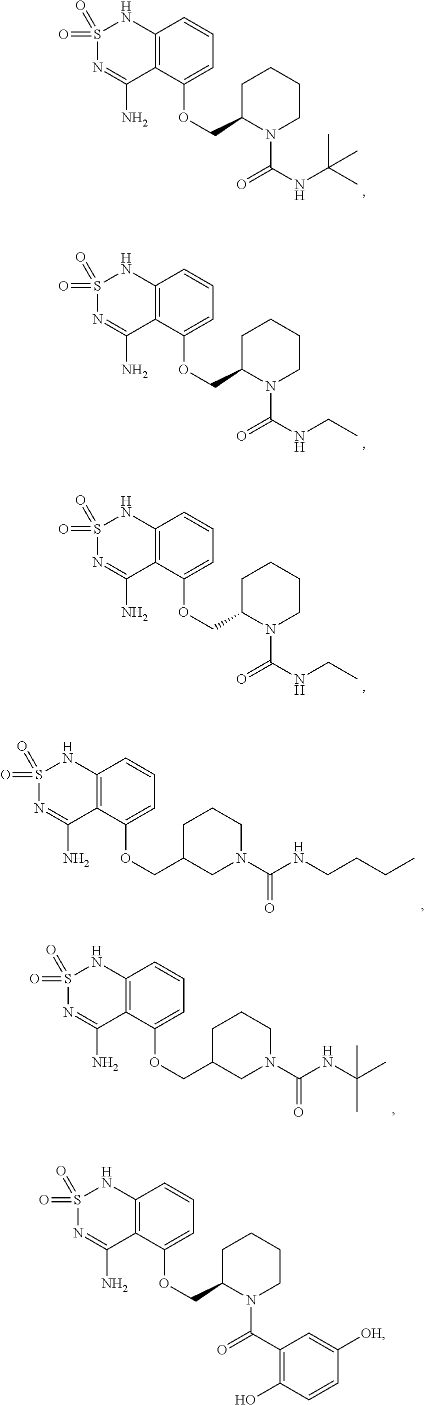 [SMILES: CC(C)(C)NC(=O)N1CCCC[C@@H]1COc1cccc2c1C(N)=NS(=O)(=O)N2 ; CCNC(=O)N1CCCC[C@@H]1COc1cccc2c1C(N)=NS(=O)(=O)N2 ; CCNC(=O)N1CCCC[C@H]1COc1cccc2c1C(N)=NS(=O)(=O)N2 ; CCCCNC(=O)N1CCCC(COc2cccc3c2C(N)=NS(=O)(=O)N3)C1 ; CC(C)(C)NC(=O)N1CCCC(COc2cccc3c2C(N)=NS(=O)(=O)N3)C1 ; NC1=NS(=O)(=O)Nc2cccc(OC[C@H]3CCCCN3C(=O)c3cc(O)ccc3O)c21]

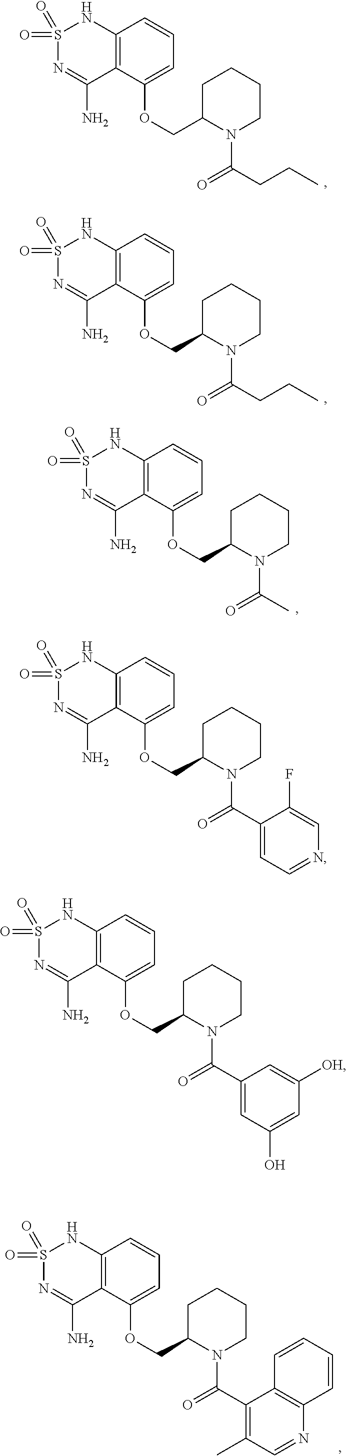 [SMILES: CCCC(=O)N1CCCCC1COc1cccc2c1C(N)=NS(=O)(=O)N2 ; CCCC(=O)N1CCCC[C@@H]1COc1cccc2c1C(N)=NS(=O)(=O)N2 ; CC(=O)N1CCCC[C@@H]1COc1cccc2c1C(N)=NS(=O)(=O)N2 ; NC1=NS(=O)(=O)Nc2cccc(OC[C@H]3CCCCN3C(=O)c3ccncc3F)c21 ; NC1=NS(=O)(=O)Nc2cccc(OC[C@H]3CCCCN3C(=O)c3cc(O)cc(O)c3)c21 ; Cc1cnc2ccccc2c1C(=O)N1CCCC[C@@H]1COc1cccc2c1C(N)=NS(=O)(=O)N2]

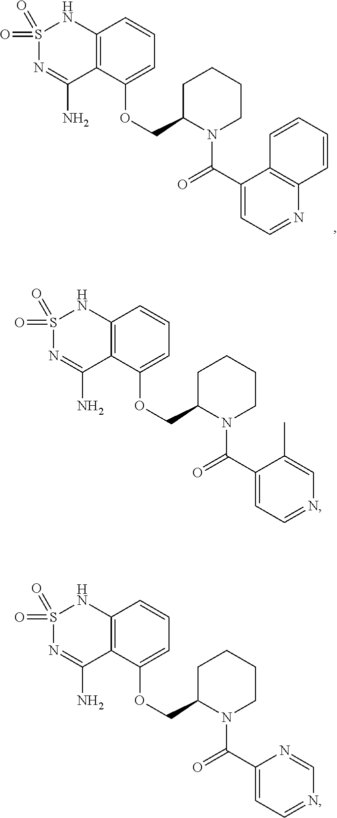 [SMILES: NC1=NS(=O)(=O)Nc2cccc(OC[C@H]3CCCCN3C(=O)c3ccnc4ccccc34)c21 ; Cc1cnccc1C(=O)N1CCCC[C@@H]1COc1cccc2c1C(N)=NS(=O)(=O)N2 ; NC1=NS(=O)(=O)Nc2cccc(OC[C@H]3CCCCN3C(=O)c3ccncn3)c21]

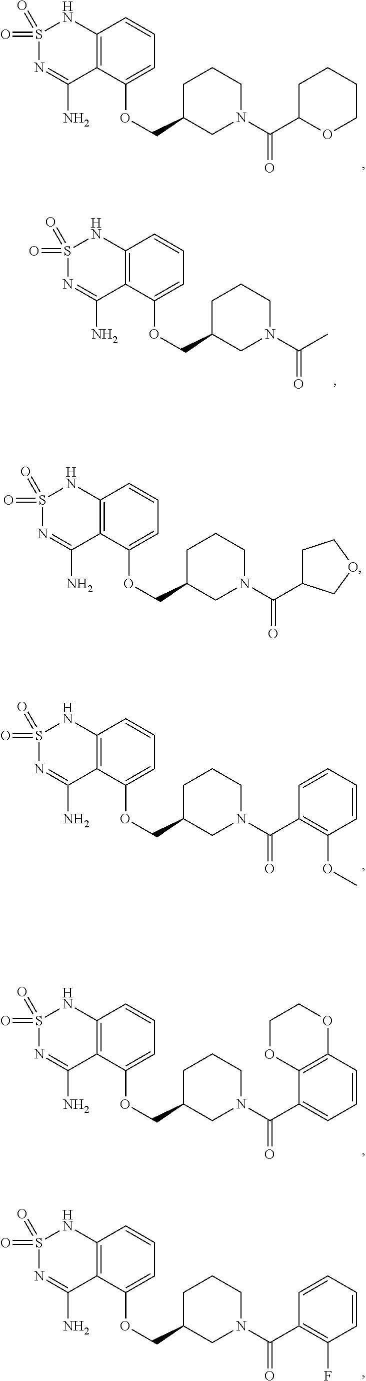 [SMILES: NC1=NS(=O)(=O)Nc2cccc(OC[C@H]3CCCN(C(=O)C4CCCCO4)C3)c21 ; CC(=O)N1CCC[C@H](COc2cccc3c2C(N)=NS(=O)(=O)N3)C1 ; NC1=NS(=O)(=O)Nc2cccc(OC[C@H]3CCCN(C(=O)C4CCOC4)C3)c21 ; COc1ccccc1C(=O)N1CCC[C@H](COc2cccc3c2C(N)=NS(=O)(=O)N3)C1 ; NC1=NS(=O)(=O)Nc2cccc(OC[C@H]3CCCN(C(=O)c4cccc5c4OCCO5)C3)c21 ; NC1=NS(=O)(=O)Nc2cccc(OC[C@H]3CCCN(C(=O)c4ccccc4F)C3)c21]

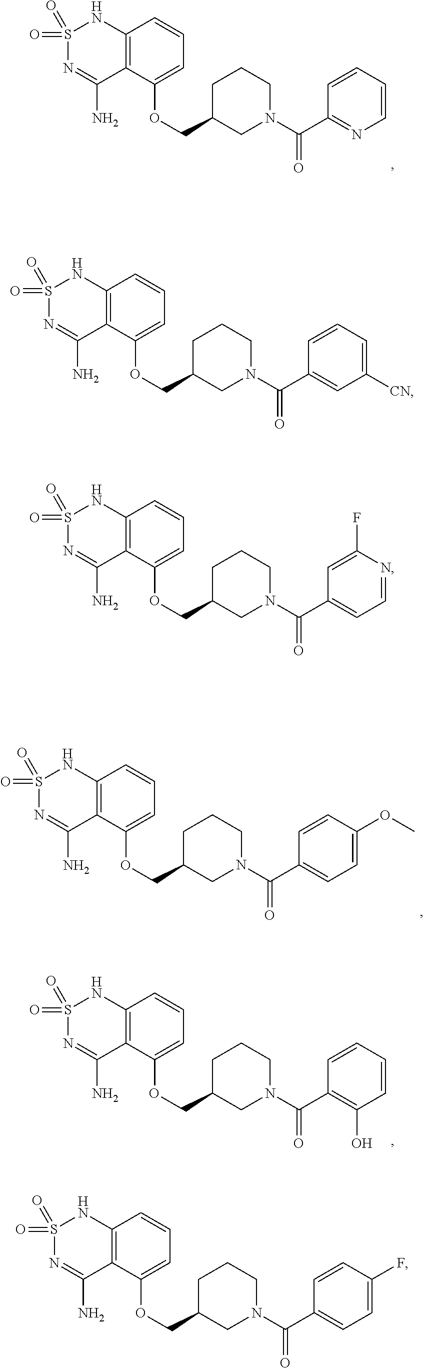 [SMILES: NC1=NS(=O)(=O)Nc2cccc(OC[C@H]3CCCN(C(=O)c4ccccn4)C3)c21 ; N#Cc1cccc(C(=O)N2CCC[C@H](COc3cccc4c3C(N)=NS(=O)(=O)N4)C2)c1 ; NC1=NS(=O)(=O)Nc2cccc(OC[C@H]3CCCN(C(=O)c4ccnc(F)c4)C3)c21 ; COc1ccc(C(=O)N2CCC[C@H](COc3cccc4c3C(N)=NS(=O)(=O)N4)C2)cc1 ; NC1=NS(=O)(=O)Nc2cccc(OC[C@H]3CCCN(C(=O)c4ccccc4O)C3)c21 ; NC1=NS(=O)(=O)Nc2cccc(OC[C@H]3CCCN(C(=O)c4ccc(F)cc4)C3)c21]

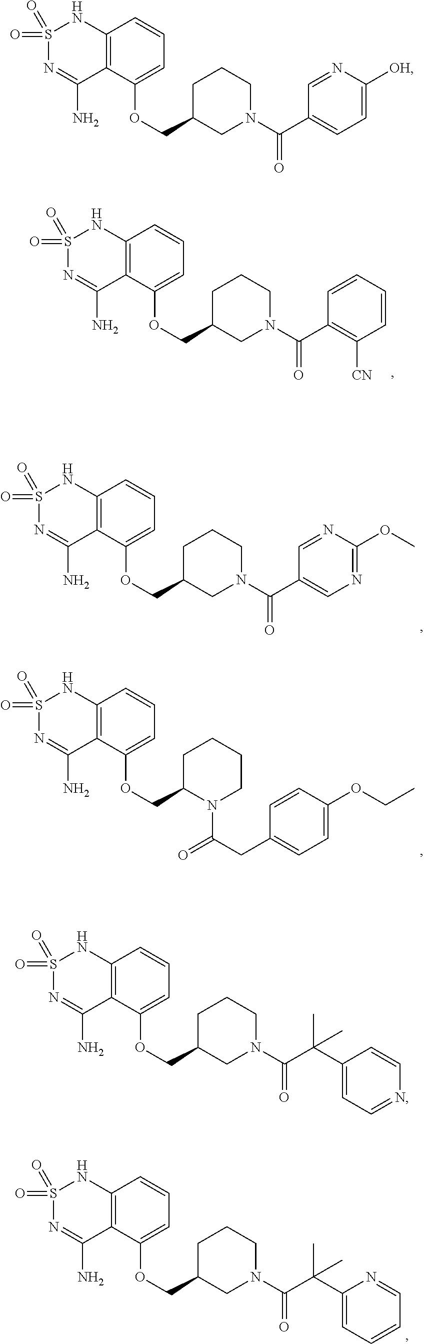 [SMILES: NC1=NS(=O)(=O)Nc2cccc(OC[C@H]3CCCN(C(=O)c4ccc(O)nc4)C3)c21 ; N#Cc1ccccc1C(=O)N1CCC[C@H](COc2cccc3c2C(N)=NS(=O)(=O)N3)C1 ; COc1ncc(C(=O)N2CCC[C@H](COc3cccc4c3C(N)=NS(=O)(=O)N4)C2)cn1 ; CCOc1ccc(CC(=O)N2CCCC[C@@H]2COc2cccc3c2C(N)=NS(=O)(=O)N3)cc1 ; CC(C)(C(=O)N1CCC[C@H](COc2cccc3c2C(N)=NS(=O)(=O)N3)C1)c1ccncc1 ; CC(C)(C(=O)N1CCC[C@H](COc2cccc3c2C(N)=NS(=O)(=O)N3)C1)c1ccccn1]

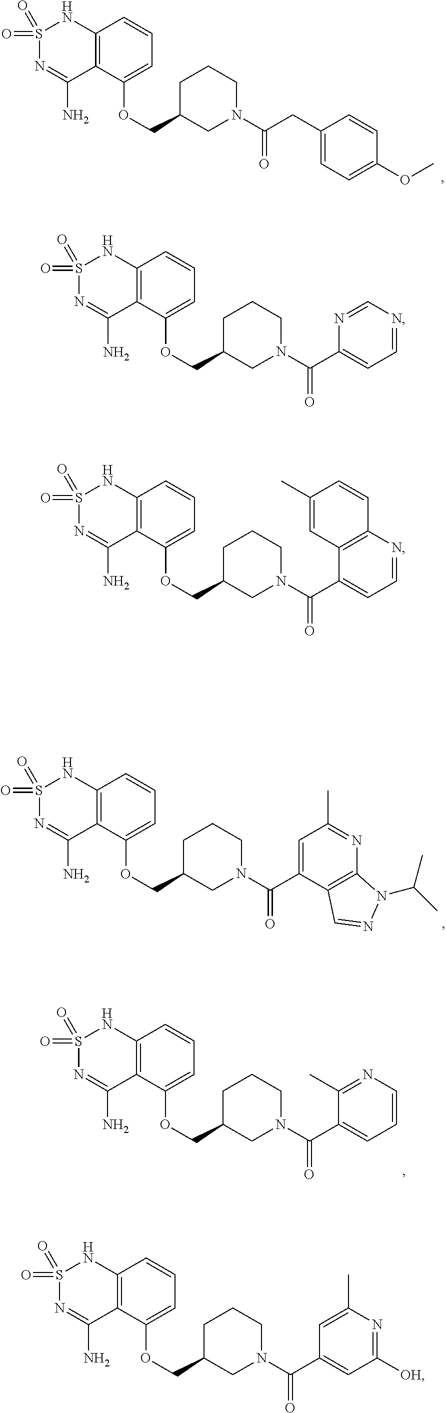 [SMILES: COc1ccc(CC(=O)N2CCC[C@H](COc3cccc4c3C(N)=NS(=O)(=O)N4)C2)cc1 ; NC1=NS(=O)(=O)Nc2cccc(OC[C@H]3CCCN(C(=O)c4ccncn4)C3)c21 ; Cc1ccc2nccc(C(=O)N3CCC[C@H](COc4cccc5c4C(N)=NS(=O)(=O)N5)C3)c2c1 ; Cc1cc(C(=O)N2CCC[C@H](COc3cccc4c3C(N)=NS(=O)(=O)N4)C2)c2cnn(C(C)C)c2n1 ; Cc1ncccc1C(=O)N1CCC[C@H](COc2cccc3c2C(N)=NS(=O)(=O)N3)C1 ; Cc1cc(C(=O)N2CCC[C@H](COc3cccc4c3C(N)=NS(=O)(=O)N4)C2)cc(O)n1]

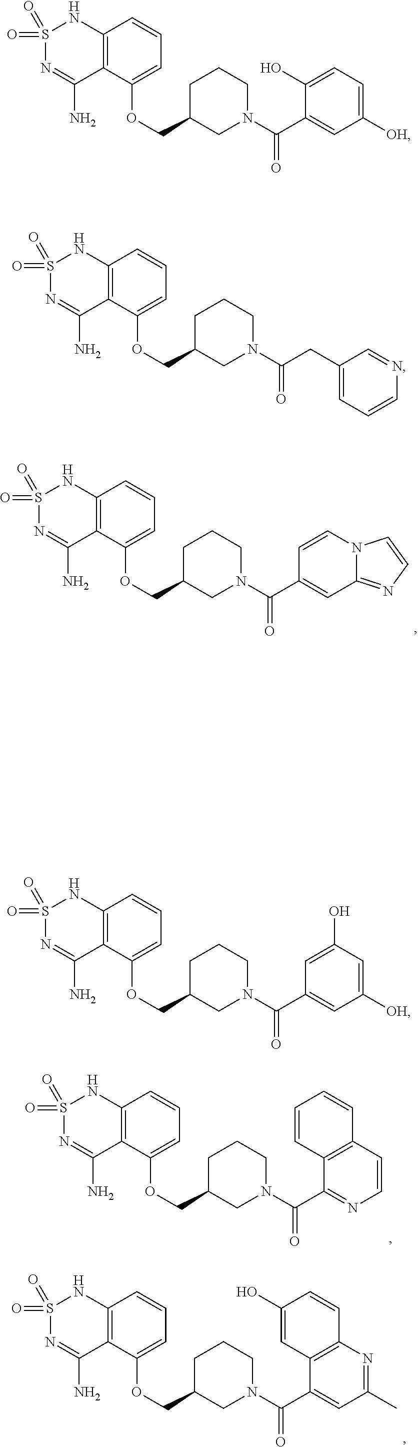 [SMILES: NC1=NS(=O)(=O)Nc2cccc(OC[C@H]3CCCN(C(=O)c4cc(O)ccc4O)C3)c21 ; NC1=NS(=O)(=O)Nc2cccc(OC[C@H]3CCCN(C(=O)Cc4cccnc4)C3)c21 ; NC1=NS(=O)(=O)Nc2cccc(OC[C@H]3CCCN(C(=O)c4ccn5ccnc5c4)C3)c21 ; NC1=NS(=O)(=O)Nc2cccc(OC[C@H]3CCCN(C(=O)c4cc(O)cc(O)c4)C3)c21 ; NC1=NS(=O)(=O)Nc2cccc(OC[C@H]3CCCN(C(=O)c4nccc5ccccc45)C3)c21 ; Cc1cc(C(=O)N2CCC[C@H](COc3cccc4c3C(N)=NS(=O)(=O)N4)C2)c2cc(O)ccc2n1]

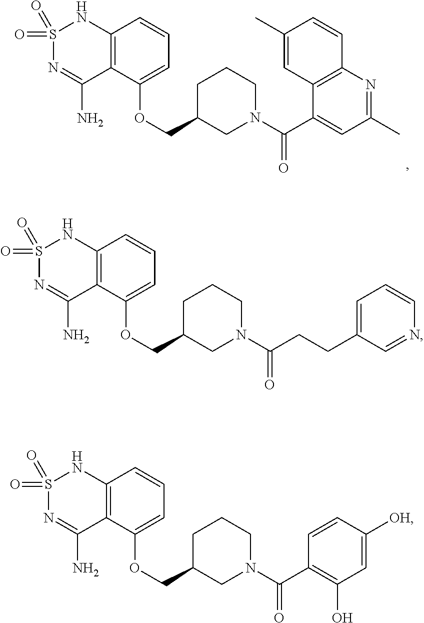 [SMILES: Cc1ccc2nc(C)cc(C(=O)N3CCC[C@H](COc4cccc5c4C(N)=NS(=O)(=O)N5)C3)c2c1 ; NC1=NS(=O)(=O)Nc2cccc(OC[C@H]3CCCN(C(=O)CCc4cccnc4)C3)c21 ; NC1=NS(=O)(=O)Nc2cccc(OC[C@H]3CCCN(C(=O)c4ccc(O)cc4O)C3)c21]

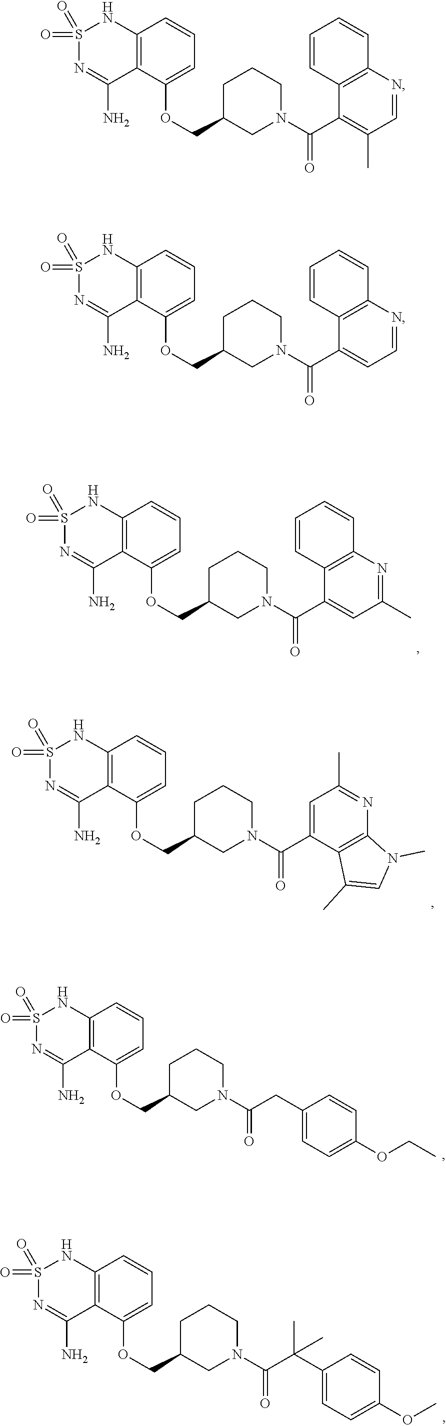 [SMILES: Cc1cnc2ccccc2c1C(=O)N1CCC[C@H](COc2cccc3c2C(N)=NS(=O)(=O)N3)C1 ; NC1=NS(=O)(=O)Nc2cccc(OC[C@H]3CCCN(C(=O)c4ccnc5ccccc45)C3)c21 ; Cc1cc(C(=O)N2CCC[C@H](COc3cccc4c3C(N)=NS(=O)(=O)N4)C2)c2ccccc2n1 ; Cc1cc(C(=O)N2CCC[C@H](COc3cccc4c3C(N)=NS(=O)(=O)N4)C2)c2c(C)cn(C)c2n1 ; CCOc1ccc(CC(=O)N2CCC[C@H](COc3cccc4c3C(N)=NS(=O)(=O)N4)C2)cc1 ; COc1ccc(C(C)(C)C(=O)N2CCC[C@H](COc3cccc4c3C(N)=NS(=O)(=O)N4)C2)cc1]

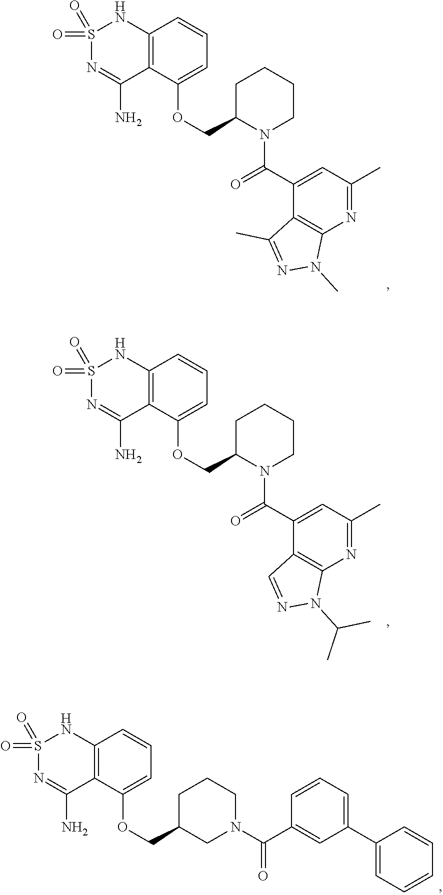 [SMILES: Cc1cc(C(=O)N2CCCC[C@@H]2COc2cccc3c2C(N)=NS(=O)(=O)N3)c2c(C)nn(C)c2n1 ; Cc1cc(C(=O)N2CCCC[C@@H]2COc2cccc3c2C(N)=NS(=O)(=O)N3)c2cnn(C(C)C)c2n1 ; NC1=NS(=O)(=O)Nc2cccc(OC[C@H]3CCCN(C(=O)c4cccc(-c5ccccc5)c4)C3)c21]

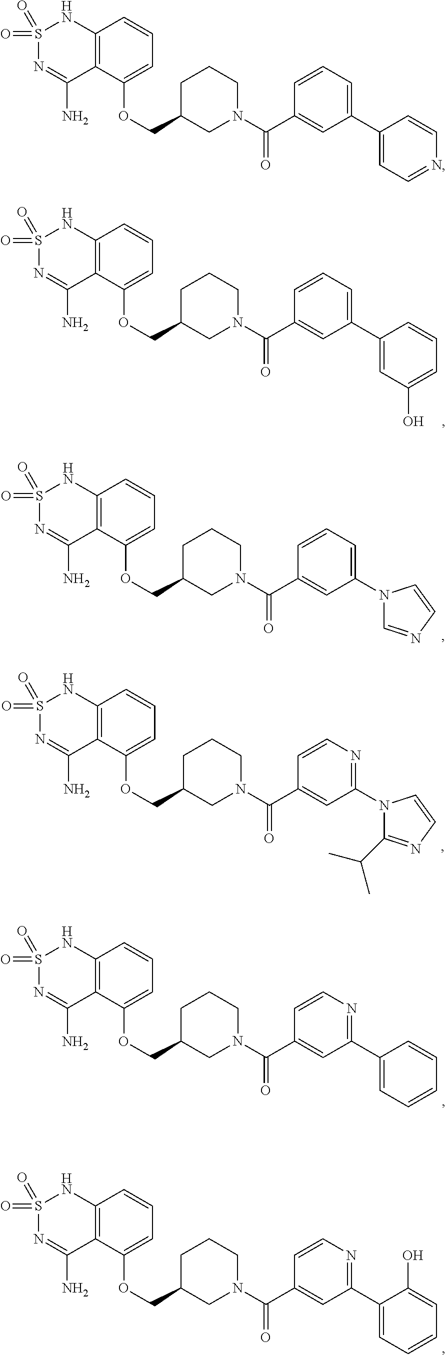

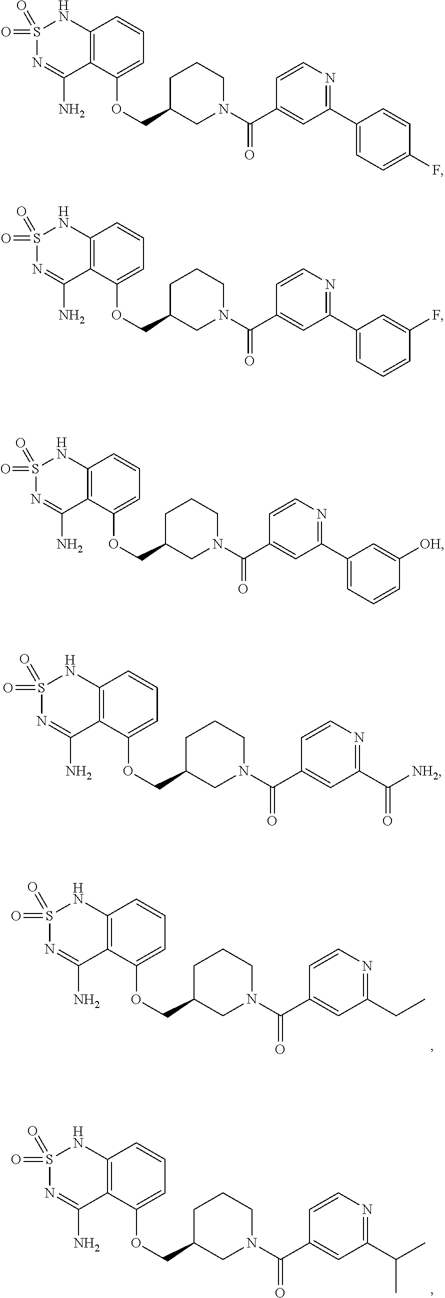 [SMILES: NC1=NS(=O)(=O)Nc2cccc(OC[C@H]3CCCN(C(=O)c4ccnc(-c5ccc(F)cc5)c4)C3)c21 ; NC1=NS(=O)(=O)Nc2cccc(OC[C@H]3CCCN(C(=O)c4ccnc(-c5cccc(F)c5)c4)C3)c21 ; NC1=NS(=O)(=O)Nc2cccc(OC[C@H]3CCCN(C(=O)c4ccnc(-c5cccc(O)c5)c4)C3)c21 ; NC(=O)c1cc(C(=O)N2CCC[C@H](COc3cccc4c3C(N)=NS(=O)(=O)N4)C2)ccn1 ; CCc1cc(C(=O)N2CCC[C@H](COc3cccc4c3C(N)=NS(=O)(=O)N4)C2)ccn1 ; CC(C)c1cc(C(=O)N2CCC[C@H](COc3cccc4c3C(N)=NS(=O)(=O)N4)C2)ccn1]

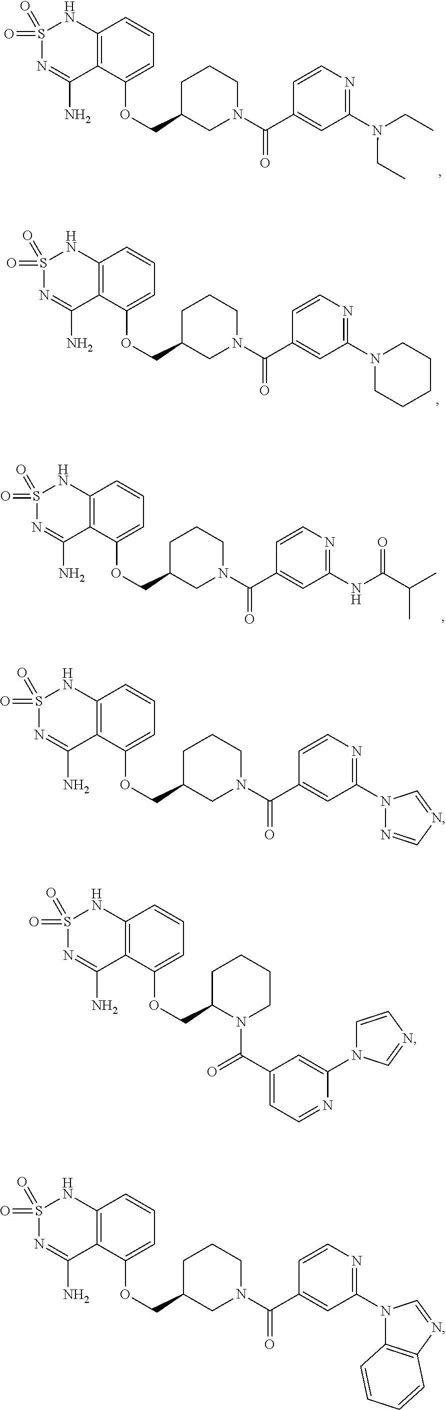 [SMILES: CCN(CC)c1cc(C(=O)N2CCC[C@H](COc3cccc4c3C(N)=NS(=O)(=O)N4)C2)ccn1 ; NC1=NS(=O)(=O)Nc2cccc(OC[C@H]3CCCN(C(=O)c4ccnc(N5CCCCC5)c4)C3)c21 ; CC(C)C(=O)Nc1cc(C(=O)N2CCC[C@H](COc3cccc4c3C(N)=NS(=O)(=O)N4)C2)ccn1 ; NC1=NS(=O)(=O)Nc2cccc(OC[C@H]3CCCN(C(=O)c4ccnc(-n5cncn5)c4)C3)c21 ; NC1=NS(=O)(=O)Nc2cccc(OC[C@H]3CCCCN3C(=O)c3ccnc(-n4ccnc4)c3)c21 ; NC1=NS(=O)(=O)Nc2cccc(OC[C@H]3CCCN(C(=O)c4ccnc(-n5cnc6ccccc65)c4)C3)c21]

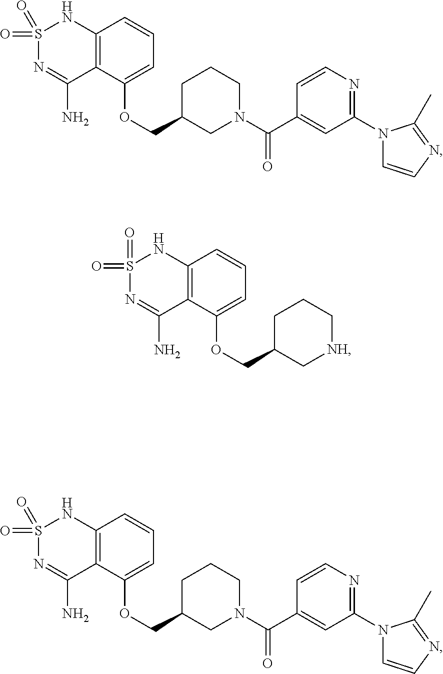 [SMILES: Cc1nccn1-c1cc(C(=O)N2CCC[C@H](COc3cccc4c3C(N)=NS(=O)(=O)N4)C2)ccn1 ; NC1=NS(=O)(=O)Nc2cccc(OC[C@H]3CCCNC3)c21 ; Cc1nccn1-c1cc(C(=O)N2CCC[C@H](COc3cccc4c3C(N)=NS(=O)(=O)N4)C2)ccn1]

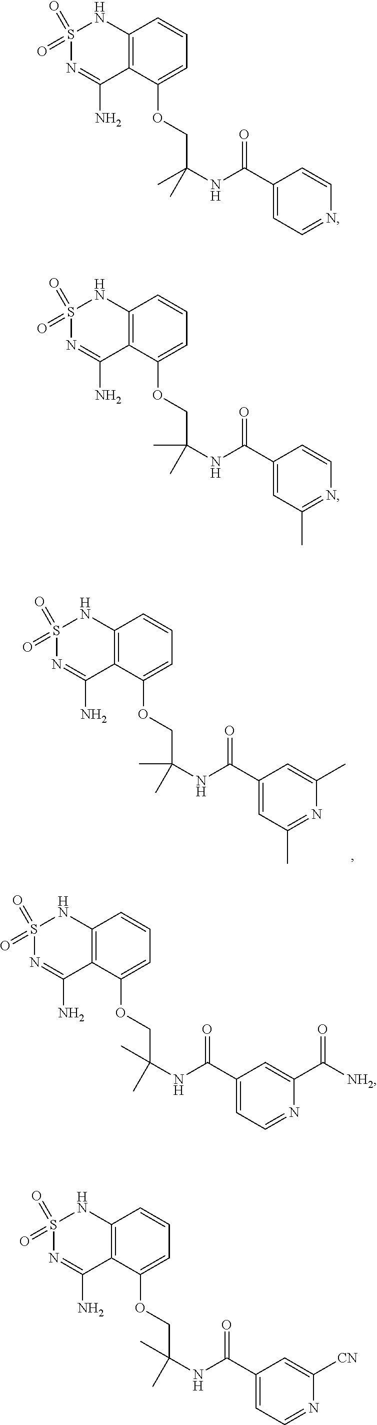 [SMILES: CC(C)(COc1cccc2c1C(N)=NS(=O)(=O)N2)NC(=O)c1ccncc1 ; Cc1cc(C(=O)NC(C)(C)COc2cccc3c2C(N)=NS(=O)(=O)N3)ccn1 ; Cc1cc(C(=O)NC(C)(C)COc2cccc3c2C(N)=NS(=O)(=O)N3)cc(C)n1 ; CC(C)(COc1cccc2c1C(N)=NS(=O)(=O)N2)NC(=O)c1ccnc(C(N)=O)c1 ; CC(C)(COc1cccc2c1C(N)=NS(=O)(=O)N2)NC(=O)c1ccnc(C#N)c1]

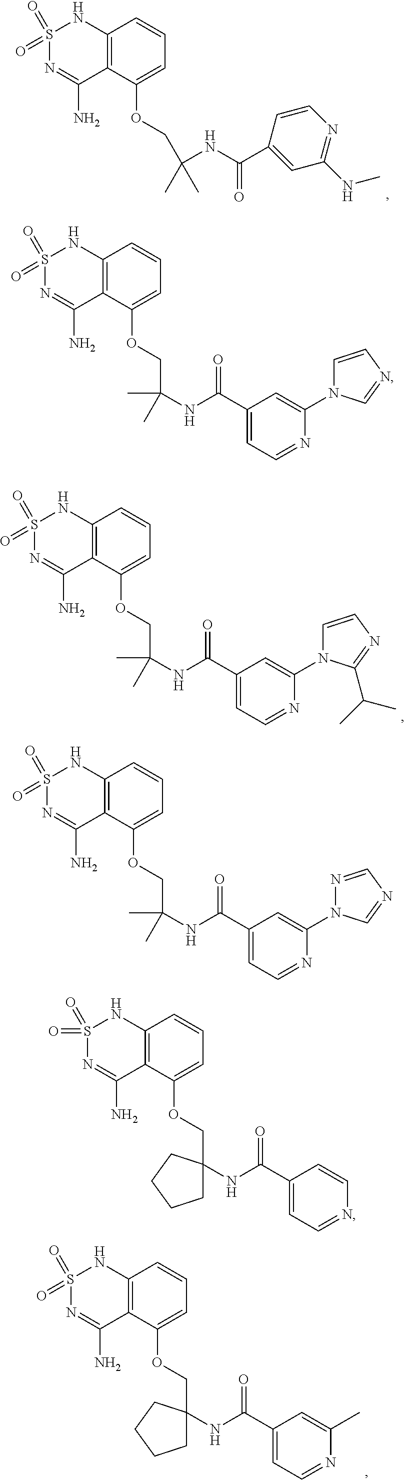 [SMILES: CNc1cc(C(=O)NC(C)(C)COc2cccc3c2C(N)=NS(=O)(=O)N3)ccn1 ; CC(C)(COc1cccc2c1C(N)=NS(=O)(=O)N2)NC(=O)c1ccnc(-n2ccnc2)c1 ; CC(C)c1nccn1-c1cc(C(=O)NC(C)(C)COc2cccc3c2C(N)=NS(=O)(=O)N3)ccn1 ; CC(C)(COc1cccc2c1C(N)=NS(=O)(=O)N2)NC(=O)c1ccnc(-n2cncn2)c1 ; NC1=NS(=O)(=O)Nc2cccc(OCC3(NC(=O)c4ccncc4)CCCC3)c21 ; Cc1cc(C(=O)NC2(COc3cccc4c3C(N)=NS(=O)(=O)N4)CCCC2)ccn1]

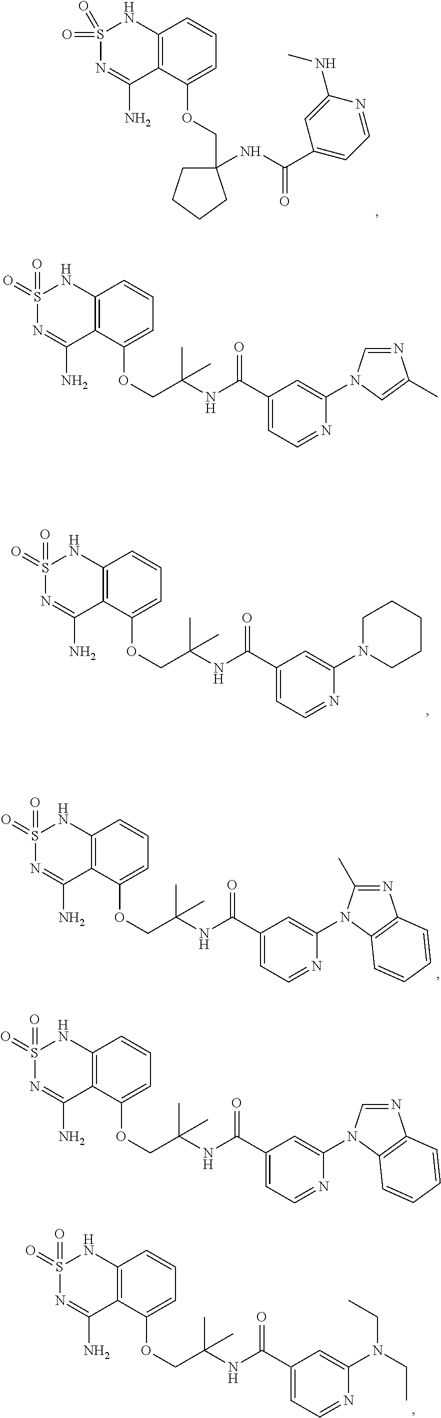 [SMILES: CNc1cc(C(=O)NC2(COc3cccc4c3C(N)=NS(=O)(=O)N4)CCCC2)ccn1 ; Cc1cn(-c2cc(C(=O)NC(C)(C)COc3cccc4c3C(N)=NS(=O)(=O)N4)ccn2)cn1 ; CC(C)(COc1cccc2c1C(N)=NS(=O)(=O)N2)NC(=O)c1ccnc(N2CCCCC2)c1 ; Cc1nc2ccccc2n1-c1cc(C(=O)NC(C)(C)COc2cccc3c2C(N)=NS(=O)(=O)N3)ccn1 ; CC(C)(COc1cccc2c1C(N)=NS(=O)(=O)N2)NC(=O)c1ccnc(-n2cnc3ccccc32)c1 ; CCN(CC)c1cc(C(=O)NC(C)(C)COc2cccc3c2C(N)=NS(=O)(=O)N3)ccn1]

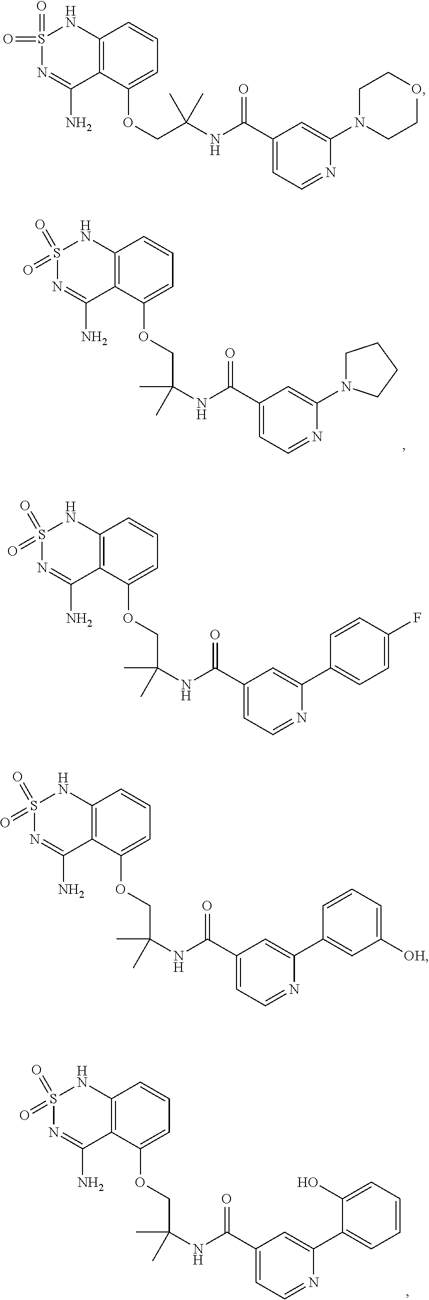 [SMILES: CC(C)(COc1cccc2c1C(N)=NS(=O)(=O)N2)NC(=O)c1ccnc(N2CCOCC2)c1 ; CC(C)(COc1cccc2c1C(N)=NS(=O)(=O)N2)NC(=O)c1ccnc(N2CCCC2)c1 ; CC(C)(COc1cccc2c1C(N)=NS(=O)(=O)N2)NC(=O)c1ccnc(-c2ccc(F)cc2)c1 ; CC(C)(COc1cccc2c1C(N)=NS(=O)(=O)N2)NC(=O)c1ccnc(-c2cccc(O)c2)c1 ; CC(C)(COc1cccc2c1C(N)=NS(=O)(=O)N2)NC(=O)c1ccnc(-c2ccccc2O)c1]

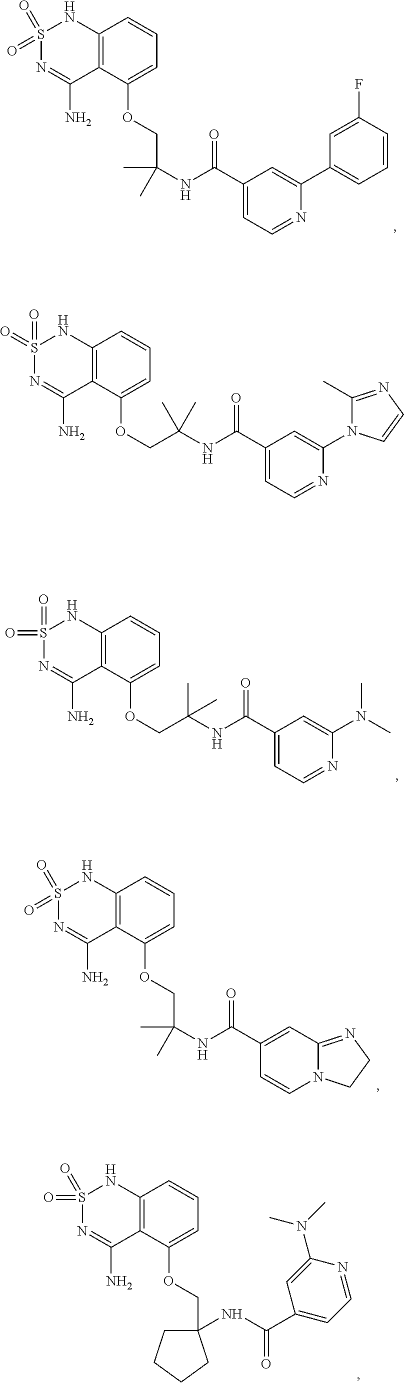 [SMILES: CC(C)(COc1cccc2c1C(N)=NS(=O)(=O)N2)NC(=O)c1ccnc(-c2cccc(F)c2)c1 ; Cc1nccn1-c1cc(C(=O)NC(C)(C)COc2cccc3c2C(N)=NS(=O)(=O)N3)ccn1 ; CN(C)c1cc(C(=O)NC(C)(C)COc2cccc3c2C(N)=NS(=O)(=O)N3)ccn1 ; CC(C)(COc1cccc2c1C(N)=NS(=O)(=O)N2)NC(=O)C1=CC2=NCCN2C=C1 ; CN(C)c1cc(C(=O)NC2(COc3cccc4c3C(N)=NS(=O)(=O)N4)CCCC2)ccn1]

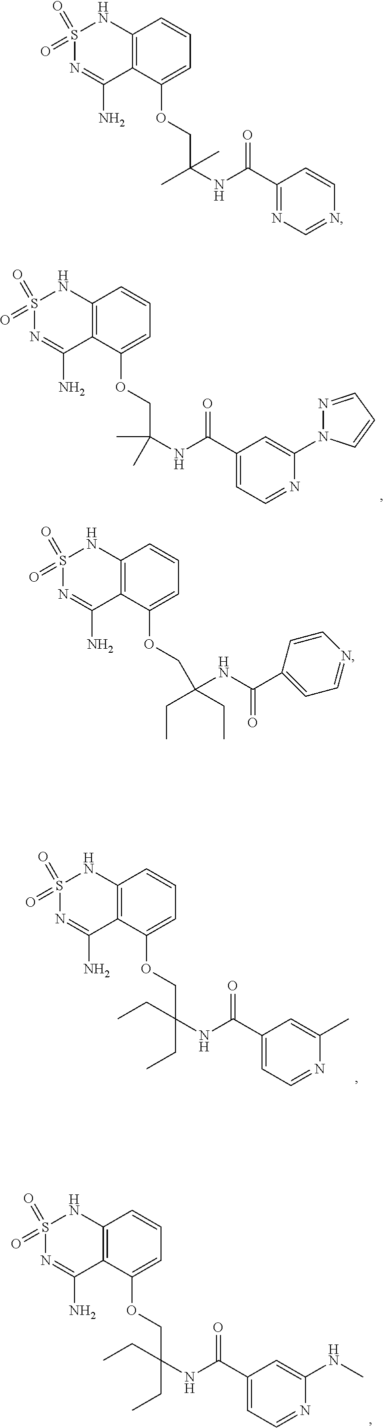 [SMILES: CC(C)(COc1cccc2c1C(N)=NS(=O)(=O)N2)NC(=O)c1ccncn1 ; CC(C)(COc1cccc2c1C(N)=NS(=O)(=O)N2)NC(=O)c1ccnc(-n2cccn2)c1 ; CCC(CC)(COc1cccc2c1C(N)=NS(=O)(=O)N2)NC(=O)c1ccncc1 ; CCC(CC)(COc1cccc2c1C(N)=NS(=O)(=O)N2)NC(=O)c1ccnc(C)c1 ; CCC(CC)(COc1cccc2c1C(N)=NS(=O)(=O)N2)NC(=O)c1ccnc(NC)c1]

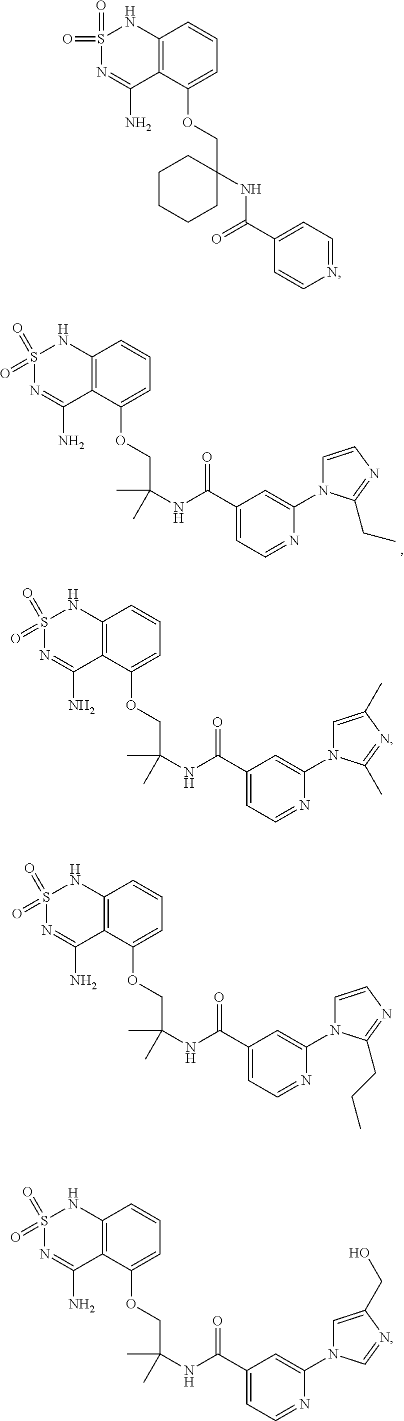 [SMILES: NC1=NS(=O)(=O)Nc2cccc(OCC3(NC(=O)c4ccncc4)CCCCC3)c21 ; CCc1nccn1-c1cc(C(=O)NC(C)(C)COc2cccc3c2C(N)=NS(=O)(=O)N3)ccn1 ; Cc1cn(-c2cc(C(=O)NC(C)(C)COc3cccc4c3C(N)=NS(=O)(=O)N4)ccn2)c(C)n1 ; CCCc1nccn1-c1cc(C(=O)NC(C)(C)COc2cccc3c2C(N)=NS(=O)(=O)N3)ccn1 ; CC(C)(COc1cccc2c1C(N)=NS(=O)(=O)N2)NC(=O)c1ccnc(-n2cnc(CO)c2)c1]

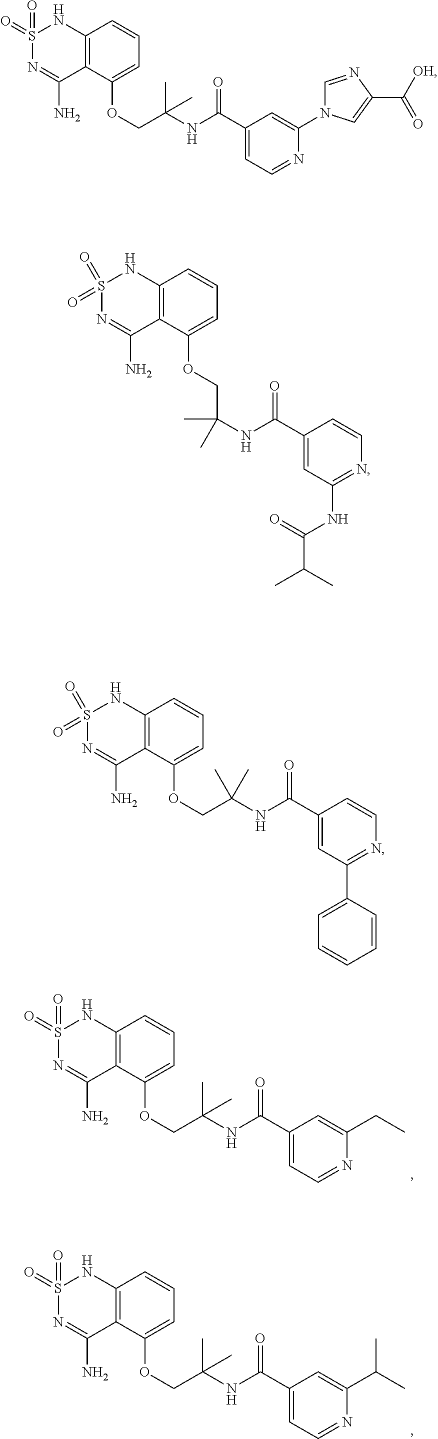 [SMILES: CC(C)(COc1cccc2c1C(N)=NS(=O)(=O)N2)NC(=O)c1ccnc(-n2cnc(C(=O)O)c2)c1 ; CC(C)C(=O)Nc1cc(C(=O)NC(C)(C)COc2cccc3c2C(N)=NS(=O)(=O)N3)ccn1 ; CC(C)(COc1cccc2c1C(N)=NS(=O)(=O)N2)NC(=O)c1ccnc(-c2ccccc2)c1 ; CCc1cc(C(=O)NC(C)(C)COc2cccc3c2C(N)=NS(=O)(=O)N3)ccn1 ; CC(C)c1cc(C(=O)NC(C)(C)COc2cccc3c2C(N)=NS(=O)(=O)N3)ccn1]

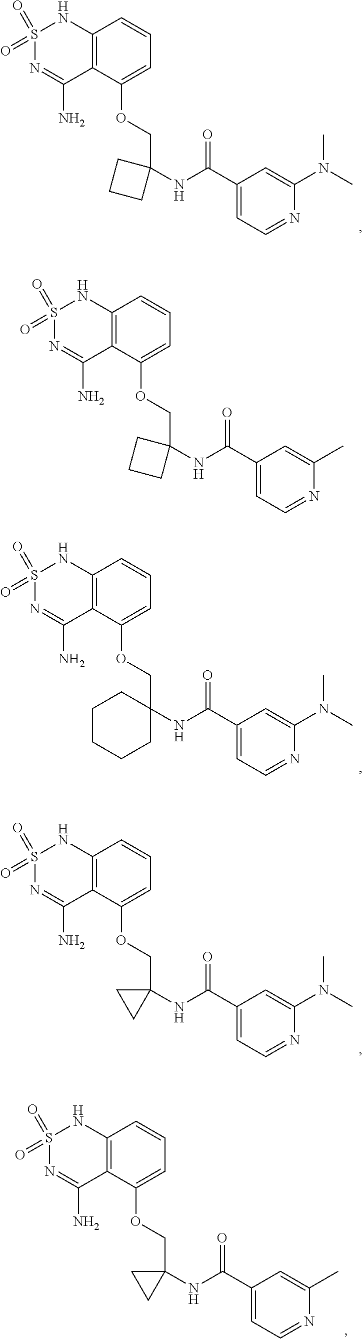 [SMILES: CN(C)c1cc(C(=O)NC2(COc3cccc4c3C(N)=NS(=O)(=O)N4)CCC2)ccn1 ; Cc1cc(C(=O)NC2(COc3cccc4c3C(N)=NS(=O)(=O)N4)CCC2)ccn1 ; CN(C)c1cc(C(=O)NC2(COc3cccc4c3C(N)=NS(=O)(=O)N4)CCCCC2)ccn1 ; CN(C)c1cc(C(=O)NC2(COc3cccc4c3C(N)=NS(=O)(=O)N4)CC2)ccn1 ; Cc1cc(C(=O)NC2(COc3cccc4c3C(N)=NS(=O)(=O)N4)CC2)ccn1]

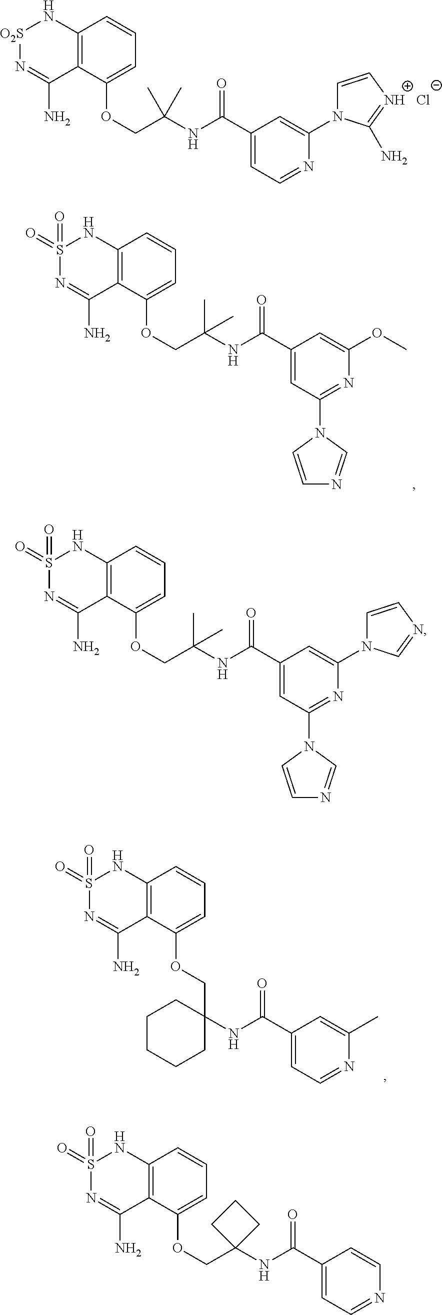

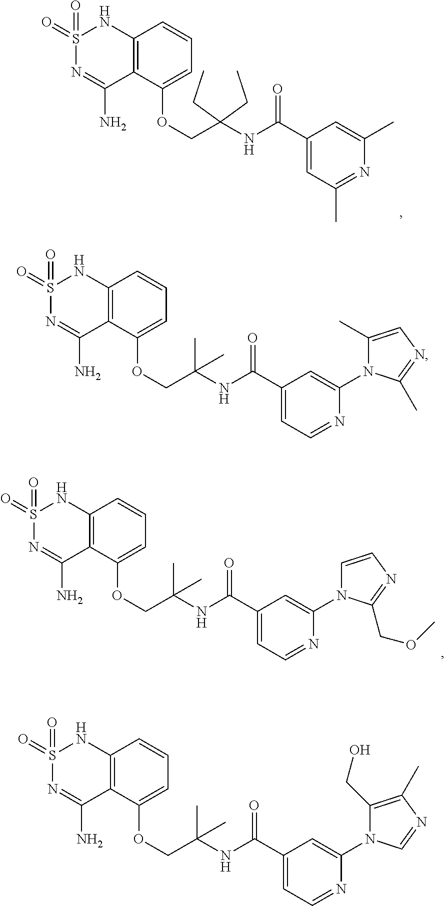 [SMILES: CCC(CC)(COc1cccc2c1C(N)=NS(=O)(=O)N2)NC(=O)c1cc(C)nc(C)c1 ; Cc1cnc(C)n1-c1cc(C(=O)NC(C)(C)COc2cccc3c2C(N)=NS(=O)(=O)N3)ccn1 ; COCc1nccn1-c1cc(C(=O)NC(C)(C)COc2cccc3c2C(N)=NS(=O)(=O)N3)ccn1 ; Cc1ncn(-c2cc(C(=O)NC(C)(C)COc3cccc4c3C(N)=NS(=O)(=O)N4)ccn2)c1CO]

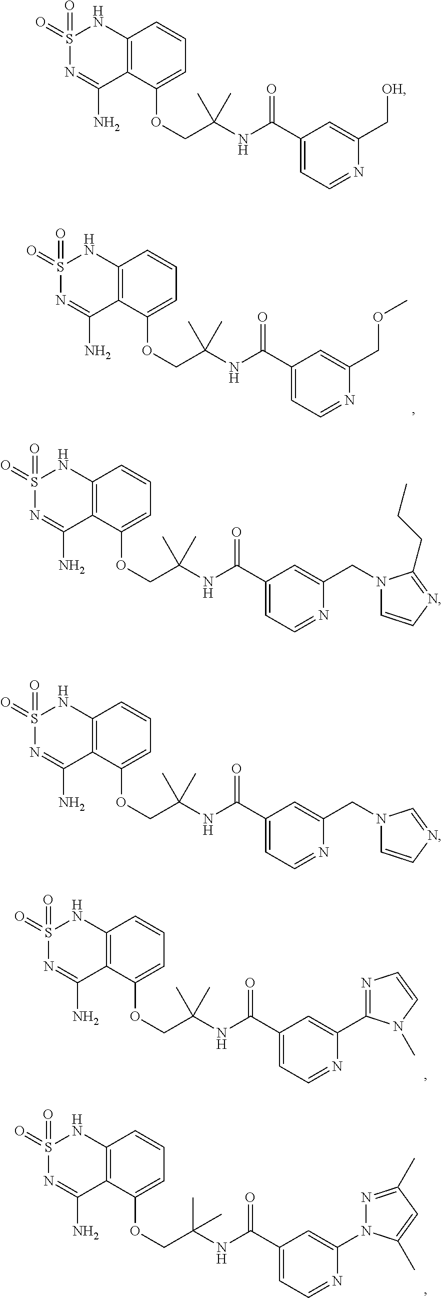 [SMILES: CC(C)(COc1cccc2c1C(N)=NS(=O)(=O)N2)NC(=O)c1ccnc(CO)c1 ; COCc1cc(C(=O)NC(C)(C)COc2cccc3c2C(N)=NS(=O)(=O)N3)ccn1 ; CCCc1nccn1Cc1cc(C(=O)NC(C)(C)COc2cccc3c2C(N)=NS(=O)(=O)N3)ccn1 ; CC(C)(COc1cccc2c1C(N)=NS(=O)(=O)N2)NC(=O)c1ccnc(Cn2ccnc2)c1 ; Cn1ccnc1-c1cc(C(=O)NC(C)(C)COc2cccc3c2C(N)=NS(=O)(=O)N3)ccn1 ; Cc1cc(C)n(-c2cc(C(=O)NC(C)(C)COc3cccc4c3C(N)=NS(=O)(=O)N4)ccn2)n1]

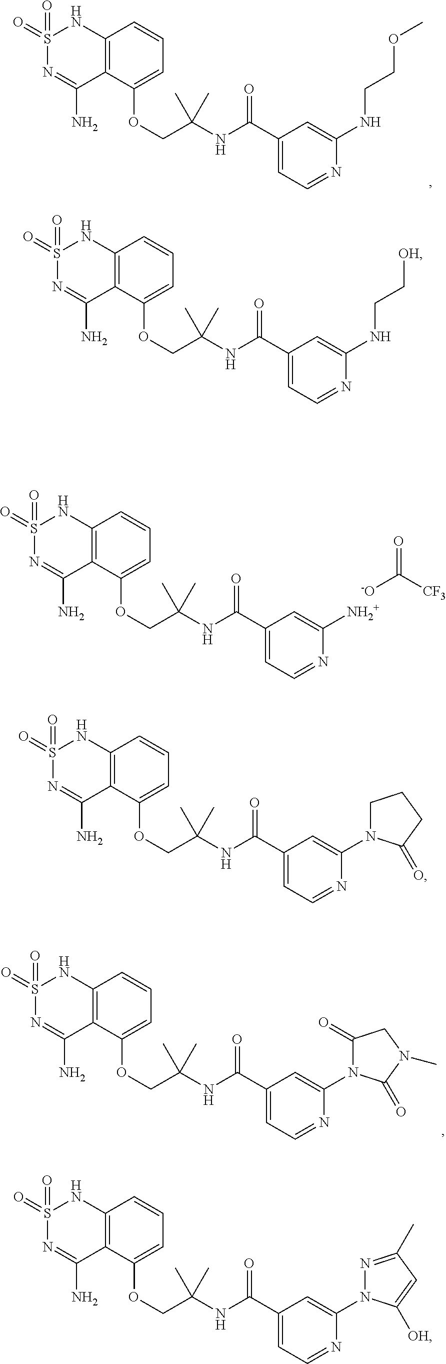

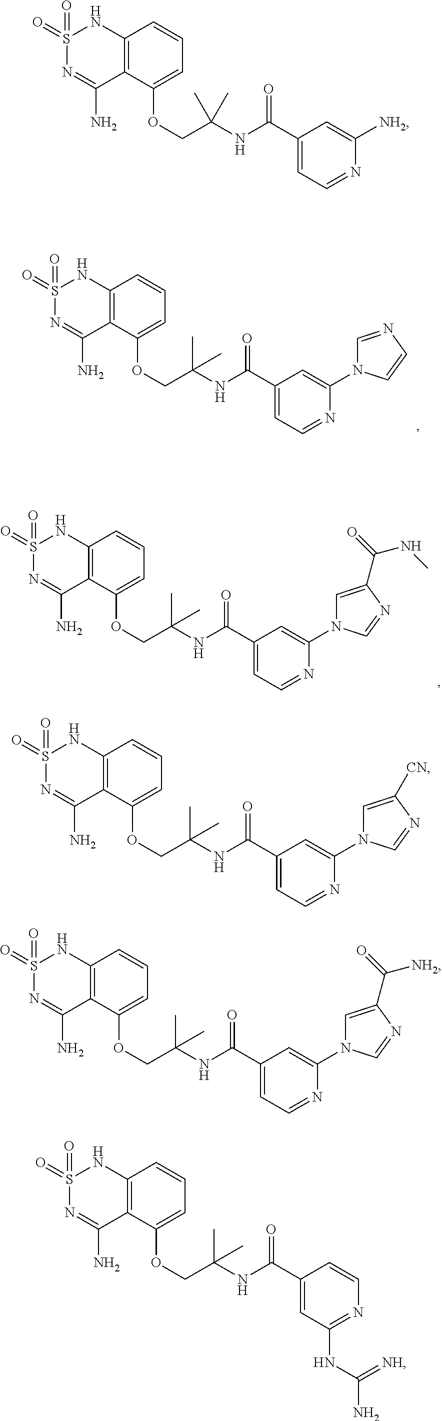 [SMILES: CC(C)(COc1cccc2c1C(N)=NS(=O)(=O)N2)NC(=O)c1ccnc(N)c1 ; CC(C)(COc1cccc2c1C(N)=NS(=O)(=O)N2)NC(=O)c1ccnc(-n2ccnc2)c1 ; CNC(=O)c1cn(-c2cc(C(=O)NC(C)(C)COc3cccc4c3C(N)=NS(=O)(=O)N4)ccn2)cn1 ; CC(C)(COc1cccc2c1C(N)=NS(=O)(=O)N2)NC(=O)c1ccnc(-n2cnc(C#N)c2)c1 ; CC(C)(COc1cccc2c1C(N)=NS(=O)(=O)N2)NC(=O)c1ccnc(-n2cnc(C(N)=O)c2)c1 ; CC(C)(COc1cccc2c1C(N)=NS(=O)(=O)N2)NC(=O)c1ccnc(NC(=N)N)c1]

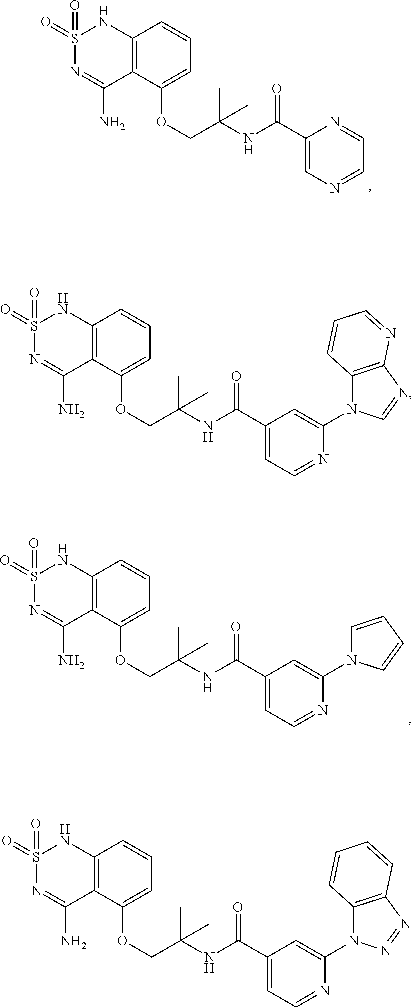 [SMILES: CC(C)(COc1cccc2c1C(N)=NS(=O)(=O)N2)NC(=O)c1cnccn1 ; CC(C)(COc1cccc2c1C(N)=NS(=O)(=O)N2)NC(=O)c1ccnc(-n2cnc3ncccc32)c1 ; CC(C)(COc1cccc2c1C(N)=NS(=O)(=O)N2)NC(=O)c1ccnc(-n2cccc2)c1 ; CC(C)(COc1cccc2c1C(N)=NS(=O)(=O)N2)NC(=O)c1ccnc(-n2nnc3ccccc32)c1]

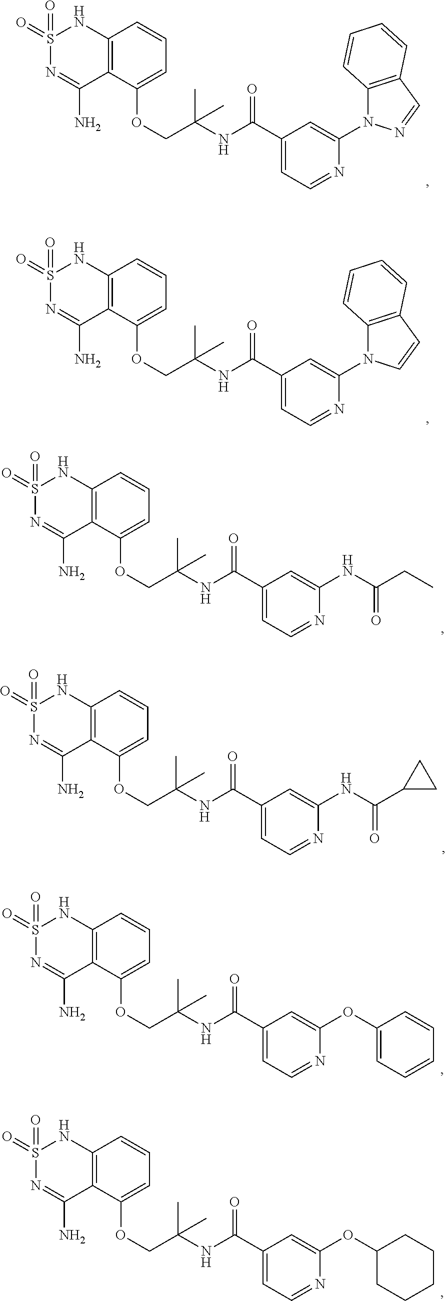 [SMILES: CC(C)(COc1cccc2c1C(N)=NS(=O)(=O)N2)NC(=O)c1ccnc(-n2ncc3ccccc32)c1 ; CC(C)(COc1cccc2c1C(N)=NS(=O)(=O)N2)NC(=O)c1ccnc(-n2ccc3ccccc32)c1 ; CCC(=O)Nc1cc(C(=O)NC(C)(C)COc2cccc3c2C(N)=NS(=O)(=O)N3)ccn1 ; CC(C)(COc1cccc2c1C(N)=NS(=O)(=O)N2)NC(=O)c1ccnc(NC(=O)C2CC2)c1 ; CC(C)(COc1cccc2c1C(N)=NS(=O)(=O)N2)NC(=O)c1ccnc(Oc2ccccc2)c1 ; CC(C)(COc1cccc2c1C(N)=NS(=O)(=O)N2)NC(=O)c1ccnc(OC2CCCCC2)c1]

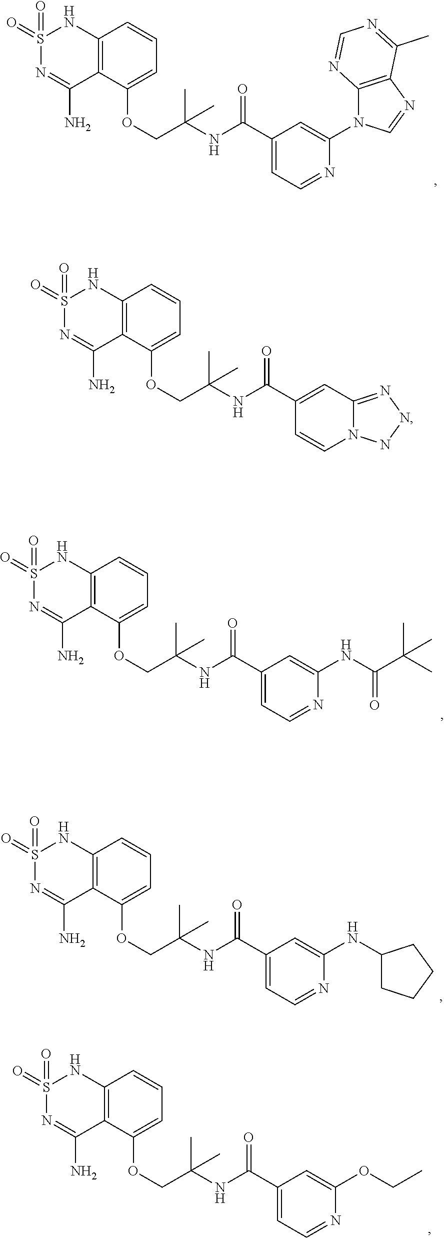

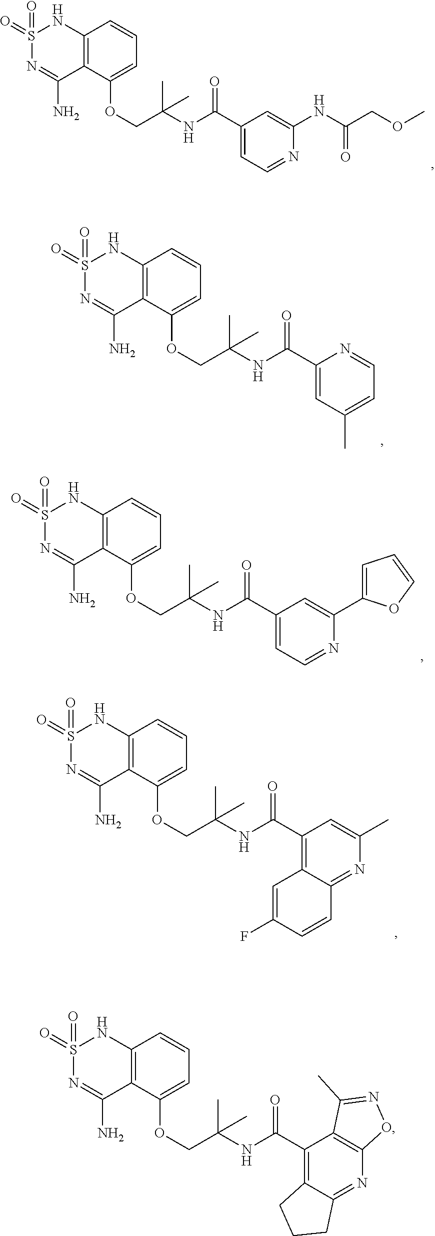 [SMILES: COCC(=O)Nc1cc(C(=O)NC(C)(C)COc2cccc3c2C(N)=NS(=O)(=O)N3)ccn1 ; Cc1ccnc(C(=O)NC(C)(C)COc2cccc3c2C(N)=NS(=O)(=O)N3)c1 ; CC(C)(COc1cccc2c1C(N)=NS(=O)(=O)N2)NC(=O)c1ccnc(-c2ccco2)c1 ; Cc1cc(C(=O)NC(C)(C)COc2cccc3c2C(N)=NS(=O)(=O)N3)c2cc(F)ccc2n1 ; Cc1noc2nc3c(c(C(=O)NC(C)(C)COc4cccc5c4C(N)=NS(=O)(=O)N5)c12)CCC3]

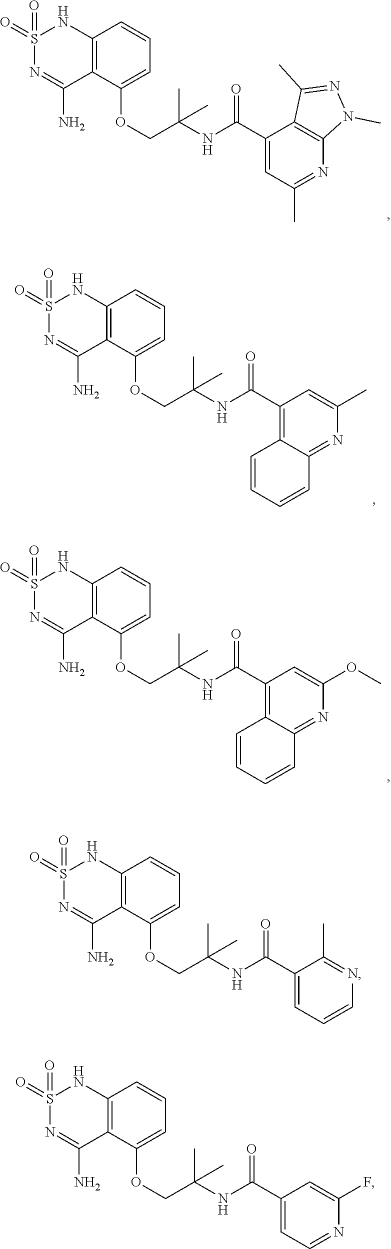 [SMILES: Cc1cc(C(=O)NC(C)(C)COc2cccc3c2C(N)=NS(=O)(=O)N3)c2c(C)nn(C)c2n1 ; Cc1cc(C(=O)NC(C)(C)COc2cccc3c2C(N)=NS(=O)(=O)N3)c2ccccc2n1 ; COc1cc(C(=O)NC(C)(C)COc2cccc3c2C(N)=NS(=O)(=O)N3)c2ccccc2n1 ; Cc1ncccc1C(=O)NC(C)(C)COc1cccc2c1C(N)=NS(=O)(=O)N2 ; CC(C)(COc1cccc2c1C(N)=NS(=O)(=O)N2)NC(=O)c1ccnc(F)c1]

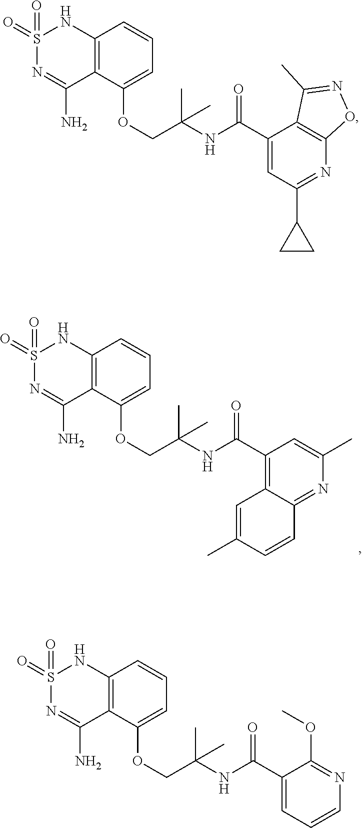 [SMILES: Cc1noc2nc(C3CC3)cc(C(=O)NC(C)(C)COc3cccc4c3C(N)=NS(=O)(=O)N4)c12 ; Cc1ccc2nc(C)cc(C(=O)NC(C)(C)COc3cccc4c3C(N)=NS(=O)(=O)N4)c2c1 ; COc1ncccc1C(=O)NC(C)(C)COc1cccc2c1C(N)=NS(=O)(=O)N2]

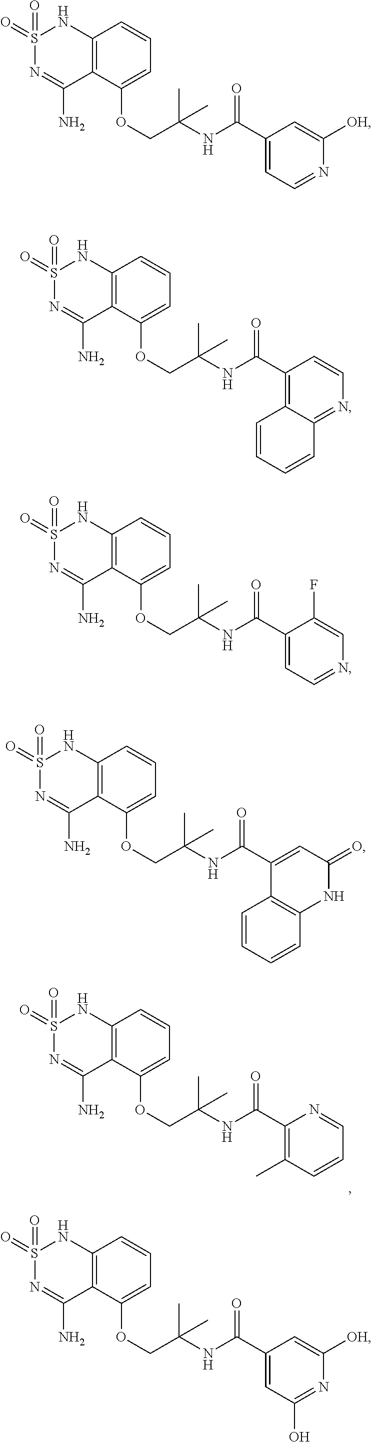 [SMILES: CC(C)(COc1cccc2c1C(N)=NS(=O)(=O)N2)NC(=O)c1ccnc(O)c1 ; CC(C)(COc1cccc2c1C(N)=NS(=O)(=O)N2)NC(=O)c1ccnc2ccccc12 ; CC(C)(COc1cccc2c1C(N)=NS(=O)(=O)N2)NC(=O)c1ccncc1F ; CC(C)(COc1cccc2c1C(N)=NS(=O)(=O)N2)NC(=O)c1cc(=O)[nH]c2ccccc12 ; Cc1cccnc1C(=O)NC(C)(C)COc1cccc2c1C(N)=NS(=O)(=O)N2 ; CC(C)(COc1cccc2c1C(N)=NS(=O)(=O)N2)NC(=O)c1cc(O)nc(O)c1]

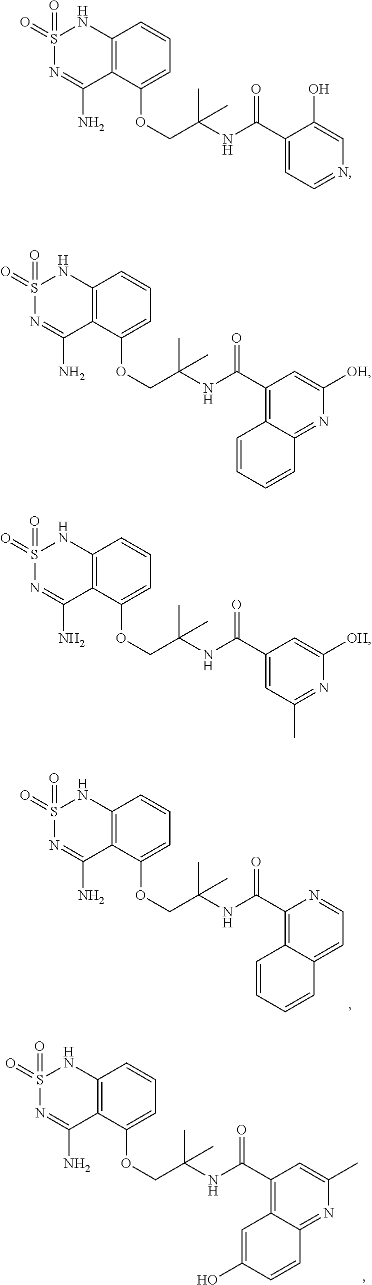 [SMILES: CC(C)(COc1cccc2c1C(N)=NS(=O)(=O)N2)NC(=O)c1ccncc1O ; CC(C)(COc1cccc2c1C(N)=NS(=O)(=O)N2)NC(=O)c1cc(O)nc2ccccc12 ; Cc1cc(C(=O)NC(C)(C)COc2cccc3c2C(N)=NS(=O)(=O)N3)cc(O)n1 ; CC(C)(COc1cccc2c1C(N)=NS(=O)(=O)N2)NC(=O)c1nccc2ccccc12 ; Cc1cc(C(=O)NC(C)(C)COc2cccc3c2C(N)=NS(=O)(=O)N3)c2cc(O)ccc2n1]

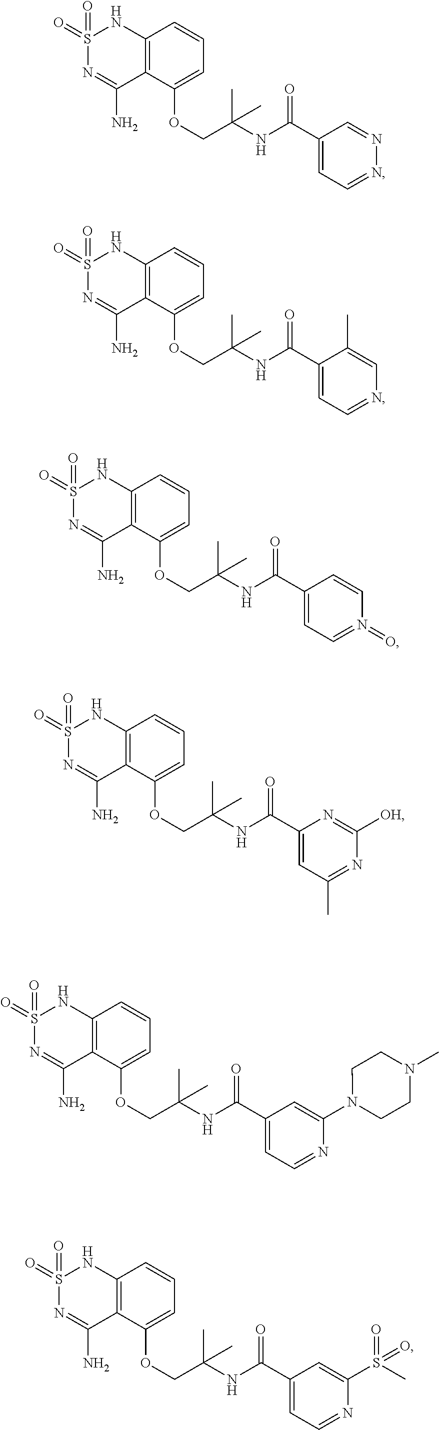 [SMILES: CC(C)(COc1cccc2c1C(N)=NS(=O)(=O)N2)NC(=O)c1ccnnc1 ; Cc1cnccc1C(=O)NC(C)(C)COc1cccc2c1C(N)=NS(=O)(=O)N2 ; CC(C)(COc1cccc2c1C(N)=NS(=O)(=O)N2)NC(=O)c1cc[n+]([O-])cc1 ; Cc1cc(C(=O)NC(C)(C)COc2cccc3c2C(N)=NS(=O)(=O)N3)nc(O)n1 ; CN1CCN(c2cc(C(=O)NC(C)(C)COc3cccc4c3C(N)=NS(=O)(=O)N4)ccn2)CC1 ; CC(C)(COc1cccc2c1C(N)=NS(=O)(=O)N2)NC(=O)c1ccnc(S(C)(=O)=O)c1]

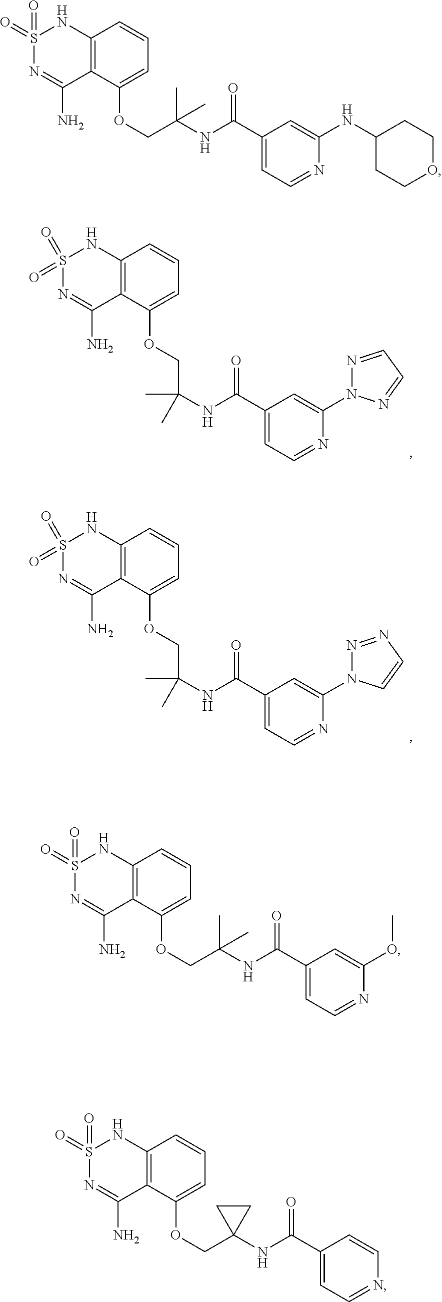 [SMILES: CC(C)(COc1cccc2c1C(N)=NS(=O)(=O)N2)NC(=O)c1ccnc(NC2CCOCC2)c1 ; CC(C)(COc1cccc2c1C(N)=NS(=O)(=O)N2)NC(=O)c1ccnc(-n2nccn2)c1 ; CC(C)(COc1cccc2c1C(N)=NS(=O)(=O)N2)NC(=O)c1ccnc(-n2ccnn2)c1 ; COc1cc(C(=O)NC(C)(C)COc2cccc3c2C(N)=NS(=O)(=O)N3)ccn1 ; NC1=NS(=O)(=O)Nc2cccc(OCC3(NC(=O)c4ccncc4)CC3)c21]

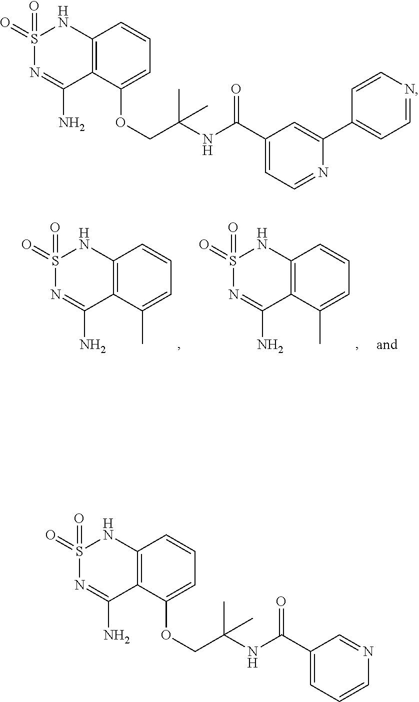

or salts thereof.

In some embodiments, the formulation includes a sweetener and a flavor modifying compound as described herein.

In some embodiments, the one or more sweetener is a natural or artificial compound. In some embodiments, the one or more sweetener is selected from the group consisting of: agave inulin, agave nectar, agave syrup, amazake, brazzein, brown rice syrup, coconut crystals, coconut sugars, coconut syrup, date sugar, fructans (also referred to as inulin fiber, fructo-oligosaccharides, or oligo-fructose), green stevia powder, stevia rebaudiana, rebaudioside A, rebaudioside B, rebaudioside C, rebaudioside D, rebaudioside E, rebaudioside F, rebaudioside I, rebaudioside H, rebaudioside L, rebaudioside K, rebaudioside J, rebaudioside N, rebaudioside O, rebaudioside M and other sweet stevia-based glycosides, stevioside, stevioside extracts, honey, Jerusalem artichoke syrup, licorice root, luo han guo (fruit, powder, or extracts), lucuma (fruit, powder, or extracts), maple sap (including, for example, sap extracted from Acer saccharum, Acer nigrum, Acer rubrum, Acer saccharinum, Acer platanoides, Acer negundo, Acer macrophyllum, Acer grandidentatum, Acer glabrum, Acer mono), maple syrup, maple sugar, walnut sap (including, for example, sap extracted from Juglans cinerea, Juglans nigra, Juglans ailatifolia, Juglans regia), birch sap (including, for example, sap extracted from Betula papyrifera, Betula alleghaniensis, Betula lenta, Betula nigra, Betula populifolia, Betula pendula), sycamore sap (such as, for example, sap extracted from Platanus occidentalis), ironwood sap (such as, for example, sap extracted from Ostrya virginiana), mascobado, molasses (such as, for example, blackstrap molasses), molasses sugar, monatin, monellin, cane sugar (also referred to as natural sugar, unrefined cane sugar, or sucrose), palm sugar, panocha, piloncillo, rapadura, raw sugar, rice syrup, sorghum, sorghum syrup, cassava syrup (also referred to as tapioca syrup), thaumatin, yacon root, malt syrup, barley malt syrup, barley malt powder, beet sugar, cane sugar, crystalline juice crystals, caramel, carbitol, carob syrup, castor sugar, hydrogenated starch hydrolates, hydrolyzed can juice, hydrolyzed starch, invert sugar, anethole, arabinogalactan, arrope, syrup, P-4000, acesulfame potassium (also referred to as acesulfame K or ace-K), alitame (also referred to as aclame), advantame, aspartame, baiyunoside, neotame, benzamide derivatives, bernadame, canderel, carrelame and other guanidine-based sweeteners, vegetable fiber, corn sugar, coupling sugars, curculin, cyclamates, cyclocarioside I, demerara, dextran, dextrin, diastatic malt, dulcin, sucrol, valzin, dulcoside A, dulcoside B, emulin, enoxolone, maltodextrin, saccharin, estragole, ethyl maltol, glucin, gluconic acid, glucono-lactone, glucosamine, glucoronic acid, glycerol, glycine, glycyphillin, glycyrrhizin, golden sugar, yellow sugar, golden syrup, granulated sugar, gynostemma, hernandulcin, isomerized liquid sugars, jallab, chicory root dietary fiber, kynurenine derivatives (including N′-formyl-kynurenine, N′-acetyl-kynurenine, 6-chloro-kynurenine), galactitol, lites se, ligicane, lycasin, lugduname, guanidine, falernum, mabinlin I, mabinlin II, maltol, maltisorb, maltodextrin, maltotriol, mannosamine, miraculin, mizuame, mogrosides (including, for example, mogroside I (including mogrosides I_{A} and I_{E}), mogroside II (including mogrosides II_{A}, II_{A1}, II_{A2}, II_{B}, and II_{E}), mogroside III (including mogroside III_{A2}, III_{E}), mogroside IV (including mogrosides IV_{A} and IV_{E}), mogroside V, and mogroside VI), 7-oxomogrosides (including, for example, 7-oxomogroside II_{E} and 7-oxomogroside V), 11-deoxymogrosides (including, for example, 11-deoxymogrosides III and V), 11-oxomogrosides (including, for example, 11-oxomogrosides I_{A}, III_{E}, IV, IV_{A}, IV_{E}, V, and VI), siamenoside, siamenoside I, isomogroside, isomogroside V, mogrol, 11-oxomogrol, neomogroside, mukurozioside, nano sugar, naringin dihydrochalcone, neohesperidine dihydrochalcone, nib sugar, nigero-oligosaccharide, norbu, orgeat syrup, osladin, pekmez, pentadin, periandrin I, perillaldehyde, perillartine, petphyllum, phenylalanine, phlomisoside I, phlorodizin, phyllodulcin, polyglycitol syrups, polypodoside A, pterocaryoside A, pterocaryoside B, rebiana, refiners syrup, rub syrup, rubusoside, selligueain A, shugr, siamenoside I, siraitia grosvenorii, soybean oligosaccharide, Splenda, SRI oxime V, steviol glycoside, steviolbioside, stevioside, strogins 1, 2, and 4, sucronic acid, sucrononate, sugar, suosan, phloridzin, superaspartame, tetrasaccharide, threitol, treacle, trilobtain, tryptophan and derivatives (6-trifluoromethyl-tryptophan, 6-chloro-D-tryptophan), vanilla sugar, volemitol, birch syrup, aspartame-acesulfame, assugrin, and combinations or blends of any two or more thereof.

Some embodiments provided herein relate to an ingestible composition, comprising a flavor modifying compound and a sweetener. In some embodiments, the ingestible composition includes a food or beverage product, animal feed product or ingredient, pharmaceutical product, nutritional product, dietary supplement, over-the-counter medication, or non-ingestible formulation, such as oral care products, hygienic products, cleansing products, or cosmetic products.

In some embodiments, the food or beverage product is for human or animal consumption. In some embodiments, the beverage is selected from the group consisting of sparkling beverages, fruit juices, fruit-flavored juices, juice drinks, nectars, vegetable juices, vegetable-flavored juices, sports drinks, energy drinks, enhanced water drinks, enhanced water with vitamins, near water drinks, coconut waters, tea type drinks, coffees, cocoa drinks, beverages containing milk components, milk alternative beverages, beverages containing cereal extracts and smoothies. In some embodiments, the beverage product includes citric acid, phosphoric acid, ascorbic acid, sodium acid sulfate, lactic acid, and/or tartaric acid; caffeine, quinine, green tea, catechins, polyphenols, green robusta coffee extract, green coffee extract, whey protein isolate, and/or potassium chloride; caramel color, Red #40, Yellow #5, Yellow #6, Blue #1, Red #3, purple carrot, black carrot juice, purple sweet potato, vegetable juice, fruit juice, beta carotene, turmeric curcumin, and/or titanium dioxide; sodium benzoate, potassium benzoate, potassium sorbate, sodium metabisulfate, sorbic acid, and/or benzoic acid; calcium disodium EDTA, alpha tocopherols, mixed tocopherols, rosemary extract, grape seed extract, resveratrol, and/or sodium hexametaphosphate; resveratrol, Co-Q10, omega 3 fatty acids, theanine, choline chloride, fibersol, inulin, taurine, panax ginseng extract, guanana extract, ginger extract, L-phenylalanine, L-carnitine, L-tartrate, D-glucoronolactone, inositol, bioflavonoids, Echinacea, ginko biloba, yerba mate, flax seed oil, garcinia cambogia rind extract, white tea extract, ribose, milk thistle extract, grape seed extract, pyrodixine HCl, cyanoobalamin, niacinamide, biotin, calcium lactate, calcium pantothenate, calcium phosphate, calcium carbonate, chromium chloride, chromium polynicotinate, cupric sulfate, folic acid, ferric pyrophosphate, iron, magnesium lactate, magnesium carbonate, magnesium sulfate, monopotassium phosphate, monosodium phosphate, phosphorus, potassium iodide, potassium phosphate, riboflavin, sodium sulfate, sodium gluconate, sodium polyphosphate, sodium bicarbonate, thiamine mononitrate, vitamin D3, vitamin A palmitate, zinc gluconate, zinc lactate, and/or zinc sulfate; ester gun, brominated vegetable oil, and/or sucrose acetate isobutyrate; sodium citrate, potassium citrate, and/or salt; ethyl alcohol, glycerine, gum Arabic, maltodextrin, modified corn starch, dextrose, natural flavor, natural flavor with other natural flavors, natural and artificial flavors, artificial flavor, silicon dioxide, magnesium carbonate, and/or tricalcium phosphate; and pectin, xanthan gum, carboxylmethylcellulose, polysorbate 60, polysorbate 80, medium chain triglycerides, cellulose gel, cellulose gum, sodium caseinate, modified food starch, and/or carrageenan.

In some embodiments, a flavor modifying compound as described herein and sweetener as described herein may be included in animal feed products or ingredients for animal feed. In some embodiments, the flavor modifying compound can be added to any animal feed. The amount of flavor modifying compound and the selection of the animal feed can vary depending upon the application. Additional components or additives can be added to the flavor modifying compound and animal feed as needed. In some embodiments, the animal feed is prepared as a pellet, a cake, a meal, a crumble, a granule, a drink, a slurry, or a slop. In some embodiments, the animal feed is prepared for a pet, for livestock, or for a ruminant or non-ruminant.

In addition to the features described above, additional features and variations will be readily apparent from the following description. It is to be understood that the following description describes typical alternatives, and is not intended to be limiting in scope. Although this disclosure is described in various exemplary alternatives and implementation as provided herein, it should be understood that the various features, aspects, and functionality described in one or more of the individual alternatives are not limited in their applicability to the particular alternative with which they are described. Instead, they can be applied alone or in various combinations to one or more of the other alternatives, whether the alternatives are described or whether the features are presented as being part of the described alternative. The breadth and scope of the present disclosure should therefore not be limited by any exemplary alternatives described herein.

### DETAILED DESCRIPTION

Embodiments disclosed herein relate generally to formulations having one or more sweetener in combination with one or more flavor modifying compound. In some embodiments, these formulations comprise non-caloric or low-caloric high-potency natural sweeteners. In some embodiments, the formulation comprises the combination of one or more sweetener and one or more flavor modifying compound that can activate the sweet receptor in vitro and impart a sweet taste enhancement. The combination formulation can be used in a variety of ingestible or non-ingestible compositions. In some embodiments, the ingestible composition comprises one or more flavor modifying compound and one or more sweeteners, which can be a natural sweetener, e.g., sucrose; or a synthetic sweetener, e.g., sucralose. In some embodiments, the natural or synthetic sweetener is a high potency sweetener. The present disclosure also relates to compositions that can improve the tastes of non-caloric or low-caloric natural and/or synthetic, high-potency sweeteners by imparting a more sugar-like taste or characteristic by utilizing natural sweeteners in conjunction with other natural or synthetic sweeteners. In some embodiments, the ingestible compositions provide a more sugar-like temporal profile, including sweetness onset and sweetness linger, and/or a more sugar-like flavor profile.

In some embodiments, the ingestible composition can be food or beverage products.

In some embodiments, the beverage can be selected from enhanced sparkling beverages, fruit juices, fruit-flavored juices, juice drinks, nectars, vegetable juices, vegetable-flavored juices, sports drinks, energy drinks, enhanced water drinks, enhanced water with vitamins, near water drinks, coconut waters, tea-type drinks, coffees, cocoa drinks, beverages containing milk components, milk alternative beverages, beverages containing cereal extracts, and smoothies.

In some embodiments, the composition can be an animal feed product or animal feed ingredient.

In some embodiments, the ingestible composition can be pharmaceutical products, nutritional products, dietary supplements, or over-the-counter medications. In some embodiments, the non-ingestible composition can be oral care products, hygienic or cosmetic products.

These and other embodiments, advantages, and features of the present disclosure are provided in part in the description that follows, and in part will be obvious from the description, or may be learned by practice of the embodiments disclosed herein. It is to be understood that both the foregoing general description and the following detailed description are exemplary and explanatory only and are not restrictive of the embodiments as described.

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

The terms “a” and “an” do not denote a limitation of quantity, but rather denote the presence of at least one of the referenced item. The term “or” or “and/or” is used as a function word to indicate that two words or expressions are to be taken together or individually. The terms “comprising”, “having”, “including”, and “containing” are to be construed as open-ended terms (for example, meaning “including, but not limited to”). The endpoints of all ranges directed to the same component or property are inclusive and independently combinable.

Many types of plants are known for producing sweet tasting compounds and/or flavor modifying compounds, and these compounds have been isolated and used as sweeteners and/or flavor modifying compounds. In some embodiments herein, the formulation of one or more sweetener in combination with one or more flavor modifying compound is obtained in whole or in part from a plant extract. By “extract”, it is meant a substance or mixture that has been taken from a plant by at least one purification or other processing step. The “plant”, as used herein, includes but is not limited to a whole plant, a plant part, a plant tissue, a plant cell, or a combination thereof. In some embodiments, the extract is obtained from a plant, a plant part, a plant tissue or a plant cell. As used herein, “plant part” or “plant tissue” refers to any part of a plant. Examples of plant parts include, but are not limited to the leaf, stem, root, tuber, seed, branch, pubescence, nodule, leaf axil, flower, pollen, stamen, pistil, petal, peduncle, stalk, stigma, style, bract, fruit, trunk, carpel, sepal, anther, ovule, pedicel, needle, cone, rhizome, spores, stolon, shoot, pericarp, endosperm, placenta, berry, stamen, sap, and leaf sheath.

The term “isolated” when referring to a compound, as used herein, means separated or isolated away from other components, ingredients, or chemicals which co-exist with the compound of interest regardless whether the other components, ingredients, or chemicals are used or generated when chemically or enzymatically synthesizing the compound of interest, or the other components, ingredients, or chemicals exist with the compound of interest in nature in its native state. In some embodiments, the term “isolated” means that the compound of interest is substantially or essentially freed from components, ingredients, or chemicals that normally accompany it in its native state by at least one purification or other processing step. Such an isolated compound may also be described as substantially pure. The term “substantially pure” as used herein describes a compound of interest that has been separated from components, ingredients, or chemicals that naturally accompany it. In some embodiments, an isolated compound is substantially pure when at least about 50%, at least about 60%, or at least about 70%, or at least about 80%, or at least about 90%, or at least about 95%, or at least about 96%, or at least about 97%, or at least about 98%, or at least about 99% of the total material (by volume, by wet or dry weight, or by mole percent or mole fraction) is the compound of interest. Purity can be measured by any appropriate method, for example by chromatography, gel electrophoresis, or HPLC analysis.

“Salt” refers to a salt of a compound, which possesses the desired activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Solvate” means a compound formed by solvation (the combination of solvent molecules with molecules or ions of the solute), or an aggregate that consists of a solute ion or molecule, for example, a compound described herein, with one or more solvent molecules. When water is the solvent, the corresponding solvate is “hydrate”.

“Vehicle” refers to a diluent, adjuvant, excipient or carrier with which a compound is combined.

As used herein, an “ingestible composition” includes any substance that, either alone or together with another substance, can be taken by mouth whether intended for consumption or not. The ingestible composition includes both “food or beverage products” and “non-edible products”. By “food or beverage products”, it is meant any edible product intended for consumption by humans or animals, including solids, semi-solids, or liquids (e.g., beverages). Ingestible compositions can also include supplements, nutraceuticals, functional food products (e.g., any fresh or processed food claimed to have a health-promoting and/or disease-preventing properties beyond the basic nutritional function of supplying nutrients), pharmaceutical and over the counter medications. The term “non-edible products” includes any product or composition that can be taken into the mouth by humans or animals for purposes other than consumption. For example, the non-edible product includes oral care products such as dentifrices and mouthwashes, cosmetic products such as sweetened lip balms and other personal care products that may or may not contain any sweetener.

As used herein, “animal” includes mammals, birds, fish, reptiles, and amphibians. Examples of animals include pets, livestock, ruminants, and non-ruminants.

A “pet” is any animal kept for companionship or protection purposes and may include dogs, cats, rabbits, reptiles, tropical fish, frogs, rodents (including rats, mice, hamsters, gerbils, and guinea pigs), potbellied pigs, and birds (including finches, canaries, and parrots). In some cases, pets may also act as working animals or sporting animals.

“Livestock” refers to domesticated animals that are raised to produce food such as meat, poultry, eggs, or dairy; to produce fiber and wearable material such as wool, fur, hair, or leather; or to perform work such as providing transportation for people or goods. Examples of livestock include, but are not limited to, cattle, horses, sheep, pigs, and chickens.

A “ruminant” is an animal that possesses a specialized stomach called a “rumen,” wherein plant-based food is fermented through bacterial action prior to further digestion. Food that has been ingested and fermented may be regurgitated and chewed again prior to the completion of digestion. The stomachs of ruminant mammals have four chambers. Ruminant animals include, but are not limited to, cows, goats, and sheep. “Non-ruminant” or “monogastric” animals, meanwhile, have stomachs with only a single compartment. Non-ruminant animals may be herbivores, omnivores, or carnivores.

In some embodiments, the ingestible composition is prepared for animal consumption as an animal feed product, or as an ingredient in animal feed. As used herein, “animal feed” means a product or composition of primarily biological origin that can be consumed by an animal and that provides nutrition for that animal. An animal feed may be a “compound feed” that is blended from multiple raw materials and is generally formulated to meet the specific needs of a target animal. An animal feed product can include pellets, cakes, meal, crumbles, granules, drinks, slurries, slops, and so forth. An animal feed can be supplemented with vitamins, minerals, preservatives, antibiotics, amino acids, pigments, and other additives; these supplements may be supplied as part of a commercial feed or as additives used by farmers, ranchers, and herders on grains purchased or grown separately.

An “ingestibly acceptable carrier or excipient” is a medium and/or composition that is used to prepare a desired dispersed dosage form of the inventive compound, in order to administer the inventive compound in a dispersed/diluted form, so that the biological effectiveness of the inventive compound is maximized. The medium and/or composition may be in any form depending on the intended use of a product, e.g., solid, semi-solid, liquid, paste, gel, lotion, cream, foamy material, suspension, solution, or any combinations thereof (such as a liquid containing solid contents). Ingestibly acceptable carriers includes many common food ingredients, such as water at neutral, acidic, or basic pH, fruit or vegetable juices, vinegar, marinades, beer, wine, natural water/fat emulsions such as milk or condensed milk, edible oils and shortenings, fatty acids and their alkyl esters, low molecular weight oligomers of propylene glycol, glyceryl esters of fatty acids, and dispersions or emulsions of such hydrophobic substances in aqueous media, salts such as sodium chloride, polysaccharides for example (but not limited to) maltodextrins, dextrin, Inulin, dextrates, polydextrose, other carbohydrates, for example (but not limited to) starch, wheat flour, corn flour, rice flour, rice starch, tapioca flour, tapioca starch, potato flour, potato starch, other flours and starches, soy flour, soy meal or other soy products, solvents such as ethanol, solid edible diluents such as vegetable powders or flours, or other liquid vehicles; dispersion or suspension aids; surface active agents; isotonic agents; thickening or emulsifying agents, preservatives; solid binders; lubricants and the like.

According to the present disclosure, a chemosensory receptor can be any receptor associated with chemosensory sensation or chemosensory ligand triggered signal transduction, e.g., via taste receptors or taste related receptors expressed in taste bud or internal organs of the body, such as gastrointestinal tract, etc. In some embodiments, a chemosensory receptor is a receptor that belongs to the 7-transmembrane receptor superfamily or G protein-coupled receptors (GPCRs). In another embodiment, a chemosensory receptor is a receptor carrying out signal transduction via one or more G proteins. In yet another embodiment, a chemosensory receptor is a receptor that belongs to family C or class C of GPCRs. In yet another embodiment, a chemosensory receptor is a receptor that belongs to the T1R family. In yet another embodiment, a chemosensory receptor is a receptor of T1R1, T1R2, T1R3, or their equivalences or variances or a combination thereof. In still another embodiment, a chemosensory receptor is a hetero-dimer of T1R2 and T1R3, or their equivalences or variances.

A “sweetener”, “sweet flavoring agent”, “sweet flavor entity”, or “sweet compound” herein refers to a compound or ingestibly acceptable salt thereof that elicits a detectable sweet flavor in a subject, e.g., fructose or a compound that activates a T1R2/T1R3 receptor in vitro.

A “flavor modifying compound” or “flavor modifier” or “flavor modifying agent” herein refers to a compound or the ingestibly acceptable salt or solvate thereof that modulates, including enhancing or potentiating, and/or inducing, the tastes of a flavoring agent in an animal or a human. An example of a flavor modifying compound is a sweet flavor enhancer.

A “flavoring agent” herein refers to a compound or the ingestibly acceptable salt or solvate thereof that induces a flavor or taste in an animal or a human. The flavoring agent can be natural, semi-synthetic, or synthetic.

A “flavor enhancer” herein refers to a compound or ingestibly acceptable salt thereof that enhances and/or multiplies the tastes of a flavoring agent, or an ingestible composition comprising the flavoring agent.

An “enhancer” herein refers to a compound, or an ingestibly acceptable salt or solvate thereof that modulates (increases) a flavor (e.g., sweetness) or the activation of a particular receptor, preferably the chemosensory, e.g., T1R2/T1R3 receptor.

A “flavor” herein refers to the perception of taste in a subject, which include sweet, sour, salty, bitter and umami. The subject may be a human or an animal.

A “sweet flavor modifier” or “sweet flavor modifying agent” herein refers to a compound or ingestibly acceptable salt or solvate thereof that modulates, including enhancing or potentiating, inducing, or blocking, the sweet taste of a sweet flavoring agents in an animal or a human. The sweet flavor modifier includes both sweet flavor enhancer and sweet flavoring agent.

A “sweet flavor enhancer” or “sweet flavor enhancing agent” herein refers to an enhancer of a sweet flavor wherein the term enhancer is the same as defined above.

A “sweet flavor modulating amount” herein refers to an amount of a flavor modifying compound that is sufficient to alter (either increase or decrease) sweet taste in an ingestible composition, or a precursor thereof, sufficiently to be perceived by a human subject. In many embodiments of the disclosure, at least about 0.001 ppm of the present compound would need to be present in order for most human subjects to perceive a modulation of the sweet flavor of an ingestible composition comprising the present compound. A broad range of concentration that would typically be employed in order to economically provide a desirable degree of sweet flavor modulation can be from about 0.001 ppm to 10,000 ppm, or a narrow range from about 0.1 ppm to about 1,000 ppm. Alternative ranges of sweet flavor modulating amounts can be from about 0.01 ppm to about 3,000 ppm, from about 0.05 ppm to about 1,500 ppm, from about 0.1 ppm to about 500 ppm, or from about 0.1 ppm to about 300 ppm.

A “sweet flavor enhancing amount” herein refers to an amount of a compound that is sufficient to enhance the taste of flavoring agents, e.g., fructose, in an ingestible composition, as perceived by an animal or a human. A broad range of a sweet flavor enhancing amount can be from about 0.001 ppm to 100 ppm, or a narrow range from about 0.1 ppm to about 10 ppm. Alternative ranges of sweet flavor enhancing amounts can be from about 0.01 ppm to about 30 ppm, from about 0.05 ppm to about 15 ppm, from about 0.1 ppm to about 5 ppm, or from about 0.1 ppm to about 3 ppm. In some embodiments, sweet flavor enhancing amount is the amount corresponding to ligand enhancing concentration(s) of a sweet flavor enhancer of the present disclosure.

A “sweet receptor modulating amount” herein refers to an amount of a compound that is sufficient to modulate (activate, enhance or block) a sweet taste receptor protein. In many embodiments of the disclosure, a sweet receptor modulating amount is at least about 10 nM, or at least about 100 nM (for example, about 0.1 μM), or at least about 1 μM, or at least about 10 μM. A “T1R2/T1R3 receptor modulating or activating amount” is an amount of compound that is sufficient to modulate or activate a T1R2/T1R3 receptor. A “sweet receptor” is a taste receptor that can be modulated by a sweet compound. Preferably a sweet receptor is a G protein coupled receptor, and more preferably the sweet receptor is a T1R2/T1R3 receptor.

The “sugar-like” characteristics of the compounds of the present disclosure include any characteristic similar to that of sucrose and include, but are not limited to, maximal response, flavor profile, temporal profile, adaptation behavior, mouthfeel, concentration/response function, tastant/and flavor/sweet taste interactions, spatial pattern selectivity, and temperature effects. These characteristics are dimensions in which the taste of sucrose is different from the tastes of other compounds. Of these, the flavor profile and temporal profile are particularly important. In a single tasting of a sweet food or beverage, differences (1) in the attributes that constitute a sweetener's flavor profile and (2) in the rates of sweetness onset and dissipation, which constitute a sweetener's temporal profile, between those observed for sucrose and other compounds can be noted.

The flavor profile of a sweetener is a quantitative profile of the relative intensities of all of the taste attributes exhibited. Such profiles often are plotted as histograms or radar plots.

In some embodiments, the compositions provided herein include a flavor modifying compound and further comprises an additional sweetener such as a sweetener described in detail below. In some embodiments, the sweetener is present in an amount from about 0.1% to about 12% by weight. In some embodiments, the sweetener is present in an amount from about 0.2% to about 10% by weight. In some embodiments, the sweetener is present in an amount from about 0.3% to about 8% by weight. In some embodiments, the sweetener is present in an amount from about 0.4% to about 6% by weight. In some embodiments, the sweetener is present in an amount from about 0.5% to about 5% by weight. In some embodiments, the sweetener is present in an amount from about 1% to about 2% by weight. In some embodiments, the sweetener is present in an amount from about 0.1% to about 5% by weight. In some embodiments, the sweetener is present in an amount from about 0.1% to about 4% by weight. In some embodiments, the sweetener is present in an amount from about 0.1% to about 3% by weight. In some embodiments, the sweetener is present in an amount from about 0.1% to about 2% by weight. In some embodiments, the sweetener is present in an amount from about 0.1% to about 1% by weight. In some embodiments, the sweetener is present in an amount from about 0.1% to about 0.5% by weight. In some embodiments, the sweetener is present in an amount from about 0.5% to about 10% by weight. In some embodiments, the sweetener is present in an amount from about 2% to about 8% by weight.

In some embodiments, the flavor modifying compound and the additional sweetener are present in a ratio of about 1:100,000 to 100,000:1, or any ratio in between. In some embodiments, the flavor modifying compound and the sweetener are present in a ratio of about 1:90,000, about 1:80,000, about 1:70,000, about 1:60,000, about 1:50,000, about 1:40,000, about 1:30,000, about 1:20,000, about 1:10,000, about 1:9,000, about 1:8,000, about 1:7,000, about 1:6,000, about 1:5,000, about 1:4,000, about 1:3,000, about 1:2,000, about 1:1,000, about 1:900, about 1:800, about 1:700, about 1:600, about 1:500, about 1:450, about 1:400, about 1:350, about 1:300, about 1:250, about 1:200, about 1:150, about 1:100, about 1:90, about 1:85, about 1:80, about 1:75, about 1:70, about 1:65, about 1:60, about 1:55, about 1:50, about 1:45, about 1:40, about 1:35, about 1:30, about 1:25, about 1:20, about 1:19, about 1:18, about 1:17, about 1:16, about 1:15, about 1:14, about 1:13, about 1:12, about 1:11, about 1:10, about 1:9, about 1:8, about 1:7, about 1:6, about 1:5, about 1:4, about 1:3, about 1:2, about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 25:1, about 30:1, about 35:1, about 40:1, about 45:1, about 50:1, about 55:1, about 60:1, about 65:1, about 70:1, about 75:1, about 80:1, about 85:1, about 90:1, about 95:1, about 100:1, about 150:1, about 200:1, about 250:1, about 300:1, about 350:1, about 400:1, about 450:1, about 500:1, about 600:1, about 700:1, about 800:1, about 900:1, about 1,000:1, about 2,000:1, about 3,000:1, about 4,000:1, about 5,000:1, about 6,000:1, about 7,000:1, about 8000:1, about 9,000:1, about 10,000:1, about 20,000:1, about 30,000:1, about 40,000:1, about 50,000:1, about 60,000:1, about 70,000:1, about 80,000:1, about 90,000:1, about 100,000:1, or any ratio in between.

### Sweeteners

Sweeteners have a wide range of chemically distinct structures and hence possess varying properties, such as, without limitation, odor, flavor, mouthfeel, and aftertaste.

Natural or artificial sweeteners for use in the formulation comprising a sweetener in combination with a flavor modifying compound include but are not limited to natural or synthetic carbohydrates or carbohydrate analogues, including monosaccharides, disaccharides, oligosaccharides, and polysaccharides, and including rare sugars, or sugars in either of the D- or L-conformations, and include, for example, sucrose, fructose, glucose, L-arabinose, L-fucose, L-glucose, L-ribose, D-arabino-hexulose, psicose, altrose, arabinose, turanose, abequose, allose, abrusoside A, aldotriose, threose, xylose, xylulose, xylo-oligosaccharide (such as xylotriose and xylobiose), lyxose, polydextrose, oligofructose, fucose, galacto-oligosaccharide, galactosamine, galactose, gentio-oligosaccharide (such as gentiobiose, gentiotriose, and gentiotetraose), dextrose, cellobiose, D-leucrose, D-psicose, D-ribose, D-tagatose, trehalose (mycose), neotrehalose, isotrehalose, raffinose, idose, tagatose, melibiose, mannan-oligosaccharide, rhamnose, ribose, ribulose, malto-oligosaccharide (such as maltotriose, maltotetraose, maltopentaose, maltohexaose, and maltoheptaose), maltose, sucrose acetate isobutyrate, dextrose, erythrose, erythrulose, deoxyribose, gulose, ketotriose, lactose, lactulose, kestose, nystose, mannose, sucralose, palatinose, polydextrose, sorbose, sugaridextrose (blended sugar), or talose, or combinations of any two or more of the aforementioned sweeteners.

The sweetener can also include, for example, sweetener compositions comprising one or more natural or synthetic carbohydrate, such as corn syrup, high fructose corn syrup, high maltose corn syrup, glucose syrup, sucralose syrup, hydrogenated glucose syrup (HGS), hydrogenated starch hydrolyzate (HSH), or other syrups or sweetener concentrates derived from natural fruit and vegetable sources, or semi-synthetic “sugar alcohol” sweeteners such as polyols. Non-limiting examples of polyols in some embodiments include erythritol, maltitol, mannitol, sorbitol, lactitol, xylitol, isomalt, propylene glycol, glycerol (glycerin), threitol, galactitol, palatinose, reduced isomalto-oligosaccharides, reduced xylo-oligosaccharides, reduced gentio-oligosaccharides, reduced maltose syrup, reduced glucose syrup, isomaltulose, maltodextrin, and the like, and sugar alcohols or any other carbohydrates or combinations thereof capable of being reduced which do not adversely affect taste.

The sweetener may be a natural or synthetic sweetener that includes, but is not limited to, agave inulin, agave nectar, agave syrup, amazake, brazzein, brown rice syrup, coconut crystals, coconut sugars, coconut syrup, date sugar, fructans (also referred to as inulin fiber, fructo-oligosaccharides, or oligo-fructose), green stevia powder, stevia rebaudiana, rebaudioside A, rebaudioside B, rebaudioside C, rebaudioside D, rebaudioside E, rebaudioside F, rebaudioside I, rebaudioside H, rebaudioside L, rebaudioside K, rebaudioside J, rebaudioside N, rebaudioside O, rebaudioside M and other sweet stevia-based glycosides, stevioside, stevioside extracts, honey, Jerusalem artichoke syrup, licorice root, luo han guo (fruit, powder, or extracts), lucuma (fruit, powder, or extracts), maple sap (including, for example, sap extracted from Acer saccharum, Acer nigrum, Acer rubrum, Acer saccharinum, Acer platanoides, Acer negundo, Acer macrophyllum, Acer grandidentatum, Acer glabrum, Acer mono), maple syrup, maple sugar, walnut sap (including, for example, sap extracted from Juglans cinerea, Juglans nigra, Juglans ailatifolia, Juglans regia), birch sap (including, for example, sap extracted from Betula papyrifera, Betula alleghaniensis, Betula lenta, Betula nigra, Betula populifolia, Betula pendula), sycamore sap (such as, for example, sap extracted from Platanus occidentalis), ironwood sap (such as, for example, sap extracted from Ostrya virginiana), mascobado, molasses (such as, for example, blackstrap molasses), molasses sugar, monatin, monellin, cane sugar (also referred to as natural sugar, unrefined cane sugar, or sucrose), palm sugar, panocha, piloncillo, rapadura, raw sugar, rice syrup, sorghum, sorghum syrup, cassava syrup (also referred to as tapioca syrup), thaumatin, yacon root, malt syrup, barley malt syrup, barley malt powder, beet sugar, cane sugar, crystalline juice crystals, caramel, carbitol, carob syrup, castor sugar, hydrogenated starch hydrolates, hydrolyzed can juice, hydrolyzed starch, invert sugar, anethole, arabinogalactan, arrope, syrup, P-4000, acesulfame potassium (also referred to as acesulfame K or ace-K), alitame (also referred to as aclame), advantame, aspartame, baiyunoside, neotame, benzamide derivatives, bernadame, canderel, carrelame and other guanidine-based sweeteners, vegetable fiber, corn sugar, coupling sugars, curculin, cyclamates, cyclocarioside I, demerara, dextran, dextrin, diastatic malt, dulcin, sucrol, valzin, dulcoside A, dulcoside B, emulin, enoxolone, maltodextrin, saccharin, estragole, ethyl maltol, glucin, gluconic acid, glucono-lactone, glucosamine, glucoronic acid, glycerol, glycine, glycyphillin, glycyrrhizin, golden sugar, yellow sugar, golden syrup, granulated sugar, gynostemma, hernandulcin, isomerized liquid sugars, jallab, chicory root dietary fiber, kynurenine derivatives (including N′-formyl-kynurenine, N′-acetyl-kynurenine, 6-chloro-kynurenine), galactitol, lites se, ligicane, lycasin, lugduname, guanidine, falernum, mabinlin I, mabinlin II, maltol, maltisorb, maltodextrin, maltotriol, mannosamine, miraculin, mizuame, mogrosides (including, for example, mogroside I (including mogrosides I_{A} and I_{E}), mogroside II (including mogrosides II_{A}, II_{A1}, II_{A2}, II_{B}, and II_{E}), mogroside Ill (including mogroside III_{A2}, III_{E}), mogroside IV (including mogrosides IV_{A} and IV_{E}), mogroside V, and mogroside VI), 7-oxomogrosides (including, for example, 7-oxomogroside II_{E} and 7-oxomogroside V), 11-deoxymogrosides (including, for example, 11-deoxymogrosides III and V), 11-oxomogrosides (including, for example, 11-oxomogrosides I_{A}, III_{E}, IV, IV_{A}, IV_{E}, V, and VI), siamenoside, siamenoside I, isomogroside, isomogroside V, mogrol, 11-oxomogrol, neomogroside, mukurozioside, nano sugar, naringin dihydrochalcone, neohesperidine dihydrochalcone, nib sugar, nigero-oligosaccharide, norbu, orgeat syrup, osladin, pekmez, pentadin, periandrin I, perillaldehyde, perillartine, petphyllum, phenylalanine, phlomisoside I, phlorodizin, phyllodulcin, polyglycitol syrups, polypodoside A, pterocaryoside A, pterocaryoside B, rebiana, refiners syrup, rub syrup, rubusoside, selligueain A, shugr, siamenoside I, siraitia grosvenorii, soybean oligosaccharide, Splenda, SRI oxime V, steviol glycoside, steviolbioside, stevioside, strogins 1, 2, and 4, sucronic acid, sucrononate, sugar, suosan, phloridzin, superaspartame, tetrasaccharide, threitol, treacle, trilobtain, tryptophan and derivatives (6-trifluoromethyl-tryptophan, 6-chloro-D-tryptophan), vanilla sugar, volemitol, birch syrup, aspartame-acesulfame, assugrin, and combinations or blends of any two or more thereof.

In still other embodiments, the sweetener can be a chemically or enzymatically modified natural high potency sweetener. Modified natural high potency sweeteners include glycosylated natural high potency sweetener such as glucosyl-, galactosyl-, or fructosyl-derivatives containing 1-50 glycosidic residues. Glycosylated natural high potency sweeteners may be prepared by enzymatic transglycosylation reaction catalyzed by various enzymes possessing transglycosylating activity. In some embodiments, the modified sweetener can be substituted or unsubstituted.

Additional sweeteners also include combinations of any two or more of any of the aforementioned sweeteners. In some embodiments, the sweetener may comprise combinations of two, three, four or five sweeteners as disclosed herein. In some embodiments, the sweetener may be a sugar. In some embodiments, the sweetener may be a combination of one or more sugars and other natural and artificial sweeteners.

One of skill in the art will recognize that any one or more of any of the aforementioned sweeteners can be combined in various ratios, amounts, or concentrations to yield a sweetener alone or a combination of two or more sweeteners, which is then combined with one or more flavor modifying compound.

One of skill in the art will recognize that the aforementioned sweeteners for use in a formulation comprising one or more sweetener and one or more flavor modifying compound are provided by way of example and are not intended to be limiting.

### Flavor Modifying Compound

In some embodiments, the flavor modifying compound is a flavor modifying compound as disclosed in U.S. Application Publication No. 2008/0306093, entitled “Modulation of Chemosensory Receptors and Ligands Associated Therewith”, filed Jun. 8, 2007, which is incorporated herein by reference in its entirety. In some embodiments, the flavor modifying compound is an embodiment of a flavor modifying compound as disclosed in U.S. Application Publication No. 2014/0094453, entitled “Sweet flavor modifier”, filed Dec. 4, 2014, which is incorporated herein by reference in its entirety. In some embodiments, the flavor modifying compound is an embodiment of a flavor modifying compound as disclosed in U.S. Application Publication No. 2014/0235624, entitled “Sweet flavor modifier”, filed Feb. 19, 2014, which is incorporated herein by reference in its entirety. In some embodiments, the flavor modifying compound is an embodiment of a flavor modifying compound as disclosed in U.S. Application Publication No. 2015/0376176, entitled “Sweet flavor modifier”, filed Aug. 14, 2015, which is incorporated herein by reference in its entirety. The above cited publications teach how to make the compounds disclosed therein.

In some embodiments, the flavor modifying compound is a compound having a cyclic thiadiazine core.

Thus, in some embodiments, provided herein is a formulation having one or more sweetener in combination with a flavor modifying compound. In certain specific embodiments, the flavor modifying compound is selected from the group consisting of:

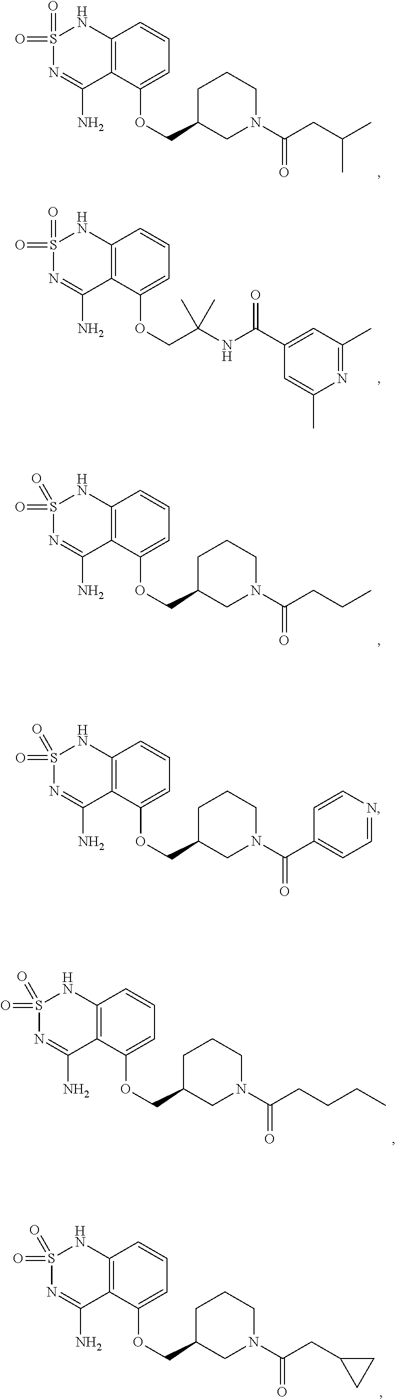

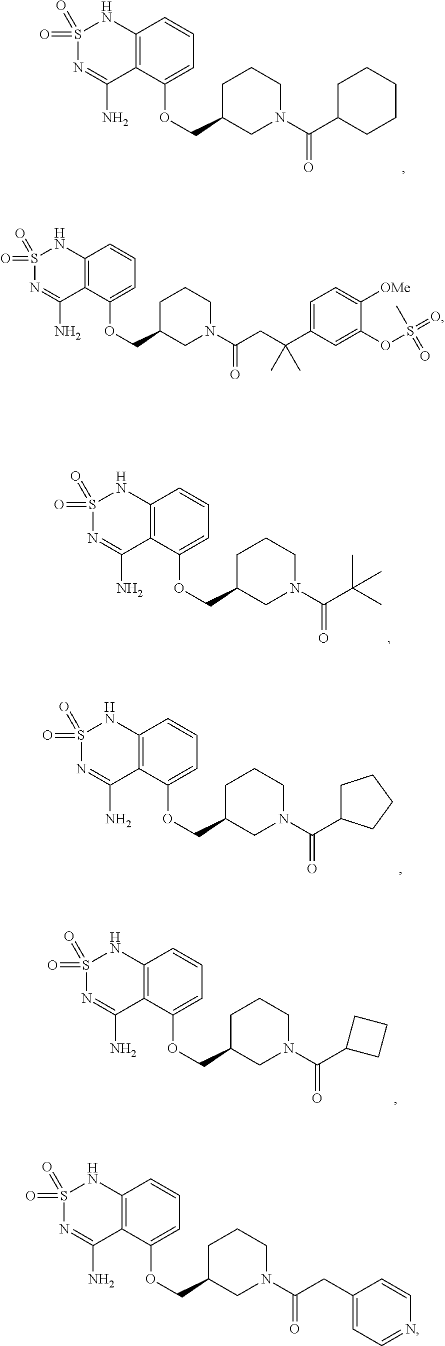

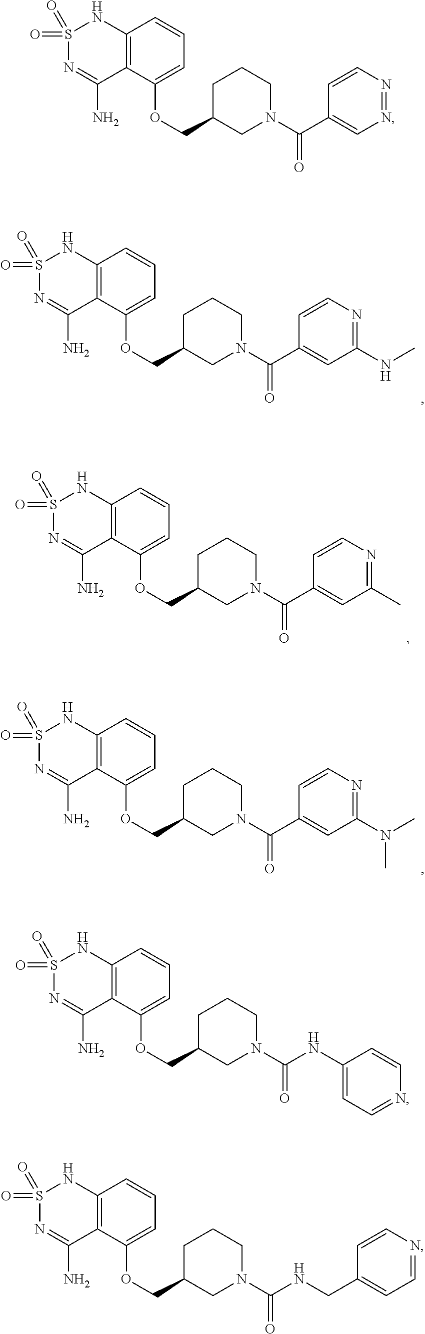

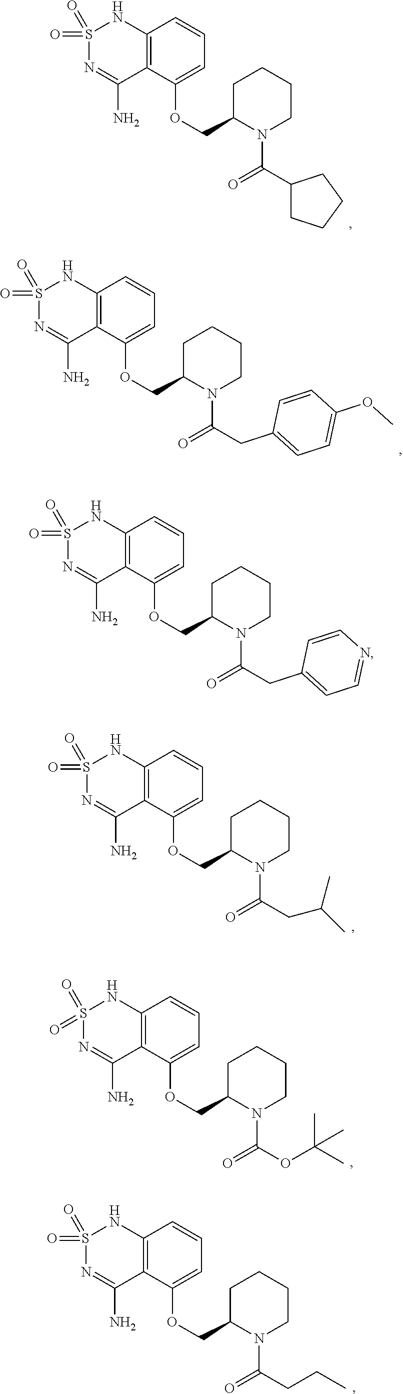

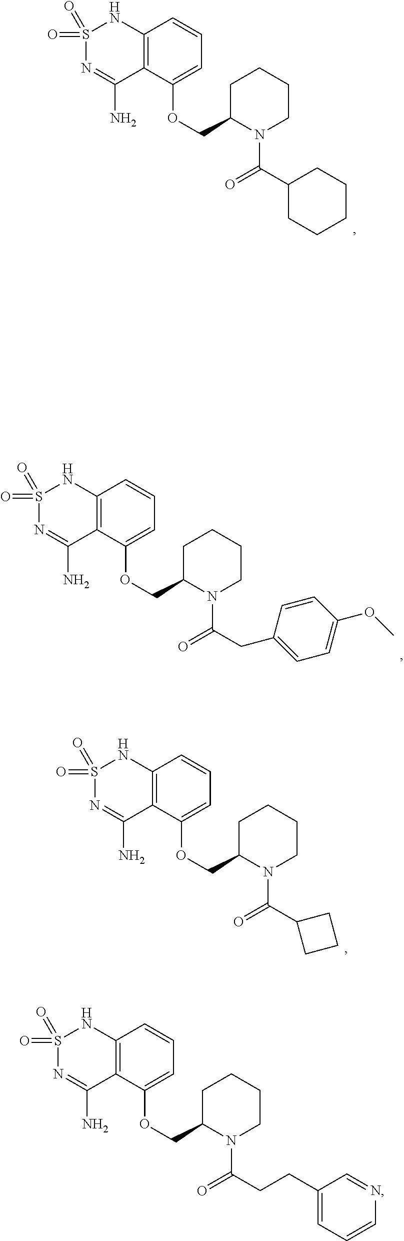

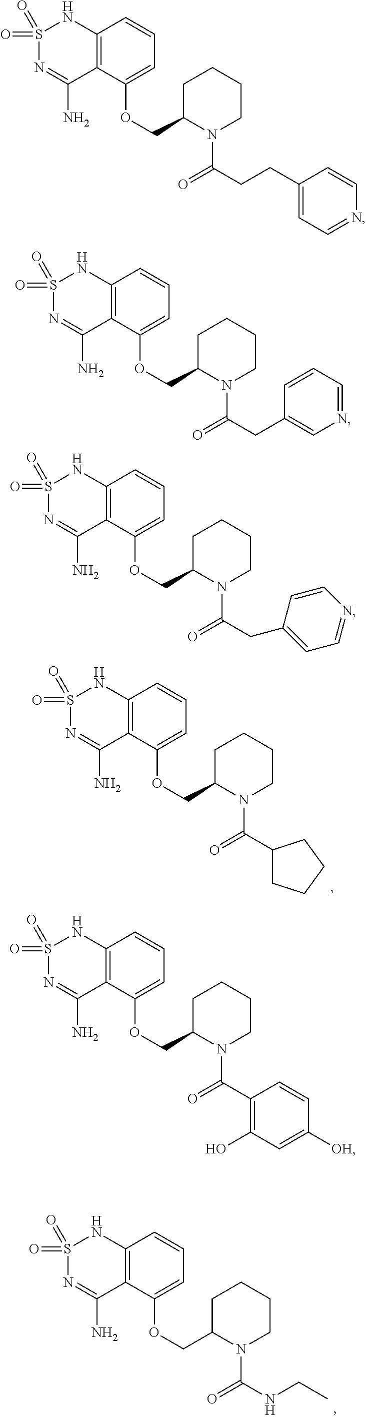 [SMILES: NC1=NS(=O)(=O)Nc2cccc(OC[C@H]3CCCCN3C(=O)CCc3ccncc3)c21 ; NC1=NS(=O)(=O)Nc2cccc(OC[C@H]3CCCCN3C(=O)Cc3cccnc3)c21 ; NC1=NS(=O)(=O)Nc2cccc(OC[C@H]3CCCCN3C(=O)Cc3ccncc3)c21 ; NC1=NS(=O)(=O)Nc2cccc(OC[C@H]3CCCCN3C(=O)C3CCCC3)c21 ; NC1=NS(=O)(=O)Nc2cccc(OC[C@H]3CCCCN3C(=O)c3ccc(O)cc3O)c21 ; CCNC(=O)N1CCCCC1COc1cccc2c1C(N)=NS(=O)(=O)N2]

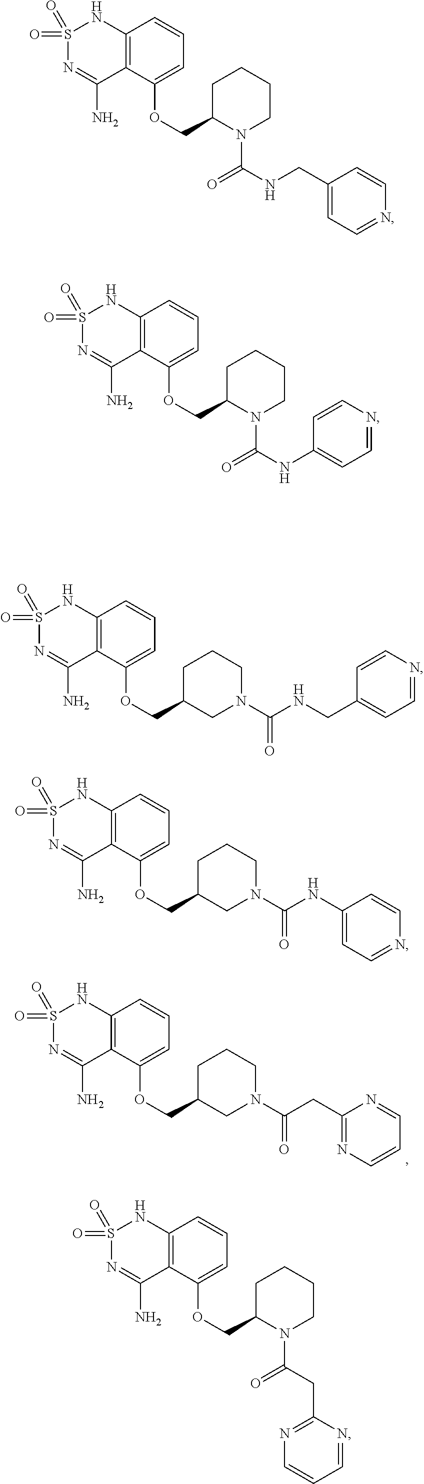 [SMILES: NC1=NS(=O)(=O)Nc2cccc(OC[C@H]3CCCCN3C(=O)NCc3ccncc3)c21 ; NC1=NS(=O)(=O)Nc2cccc(OC[C@H]3CCCCN3C(=O)Nc3ccncc3)c21 ; NC1=NS(=O)(=O)Nc2cccc(OC[C@H]3CCCN(C(=O)NCc4ccncc4)C3)c21 ; NC1=NS(=O)(=O)Nc2cccc(OC[C@H]3CCCN(C(=O)Nc4ccncc4)C3)c21 ; NC1=NS(=O)(=O)Nc2cccc(OC[C@H]3CCCN(C(=O)Cc4ncccn4)C3)c21 ; NC1=NS(=O)(=O)Nc2cccc(OC[C@H]3CCCCN3C(=O)Cc3ncccn3)c21]

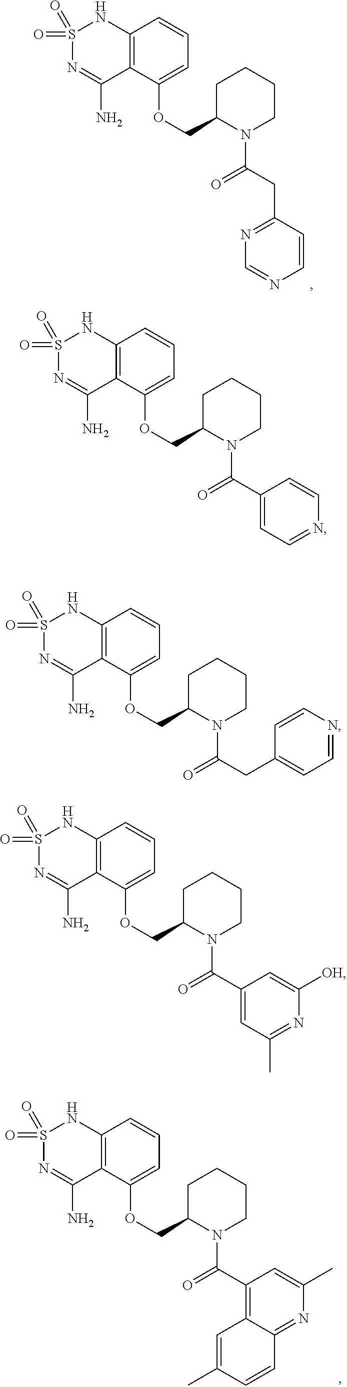 [SMILES: NC1=NS(=O)(=O)Nc2cccc(OC[C@H]3CCCCN3C(=O)Cc3ccncn3)c21 ; "," ; NC1=NS(=O)(=O)Nc2cccc(OC[C@H]3CCCCN3C(=O)c3ccncc3)c21 ; NC1=NS(=O)(=O)Nc2cccc(OC[C@H]3CCCCN3C(=O)Cc3ccncc3)c21 ; Cc1cc(C(=O)N2CCCC[C@@H]2COc2cccc3c2C(N)=NS(=O)(=O)N3)cc(O)n1 ; Cc1ccc2nc(C)cc(C(=O)N3CCCC[C@@H]3COc3cccc4c3C(N)=NS(=O)(=O)N4)c2c1]

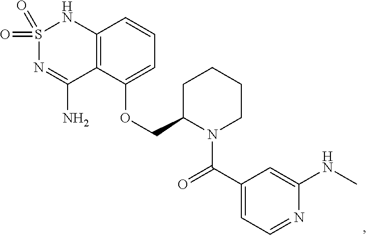 [SMILES: CNc1cc(C(=O)N2CCCC[C@@H]2COc2cccc3c2C(N)=NS(=O)(=O)N3)ccn1]

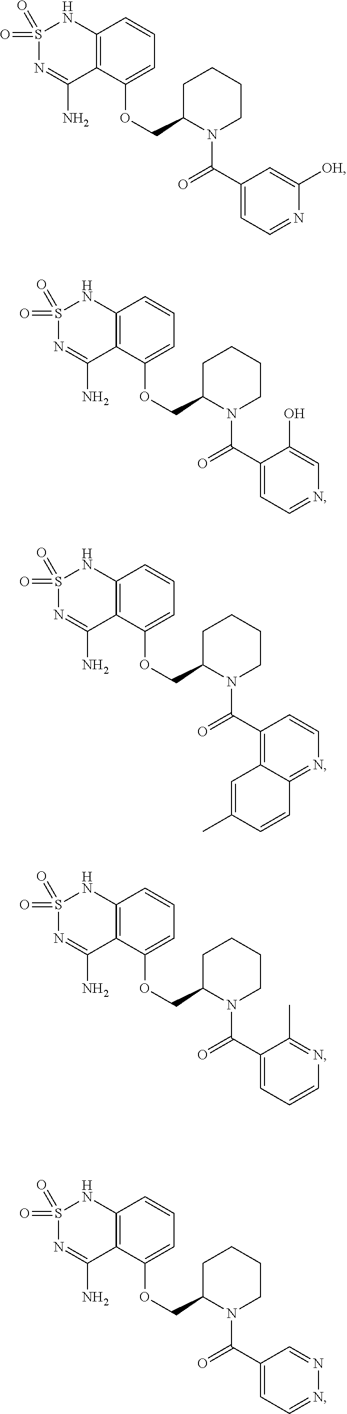 [SMILES: NC1=NS(=O)(=O)Nc2cccc(OC[C@H]3CCCCN3C(=O)c3ccnc(O)c3)c21 ; NC1=NS(=O)(=O)Nc2cccc(OC[C@H]3CCCCN3C(=O)c3ccncc3O)c21 ; Cc1ccc2nccc(C(=O)N3CCCC[C@@H]3COc3cccc4c3C(N)=NS(=O)(=O)N4)c2c1 ; Cc1ncccc1C(=O)N1CCCC[C@@H]1COc1cccc2c1C(N)=NS(=O)(=O)N2 ; NC1=NS(=O)(=O)Nc2cccc(OC[C@H]3CCCCN3C(=O)c3ccnnc3)c21]

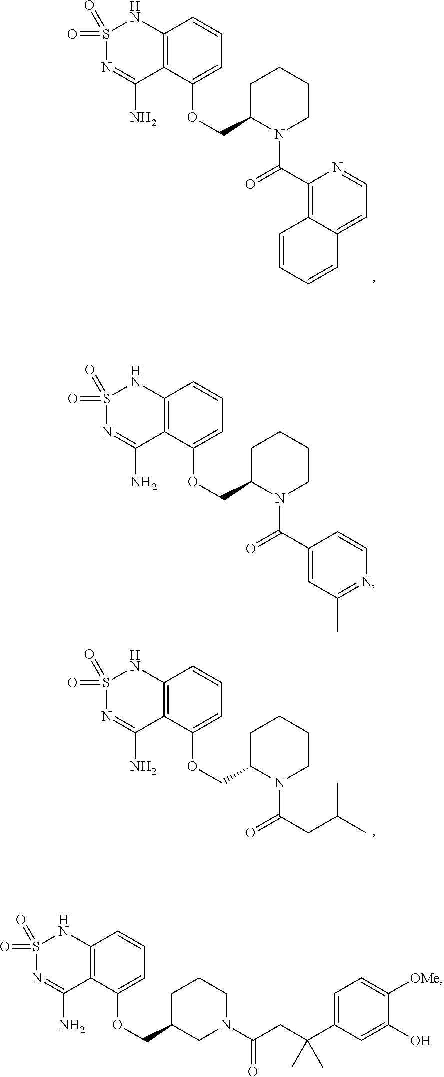 [SMILES: NC1=NS(=O)(=O)Nc2cccc(OC[C@H]3CCCCN3C(=O)c3nccc4ccccc34)c21 ; Cc1cc(C(=O)N2CCCC[C@@H]2COc2cccc3c2C(N)=NS(=O)(=O)N3)ccn1 ; CC(C)CC(=O)N1CCCC[C@H]1COc1cccc2c1C(N)=NS(=O)(=O)N2 ; COc1ccc(C(C)(C)CC(=O)N2CCC[C@H](COc3cccc4c3C(N)=NS(=O)(=O)N4)C2)cc1O]

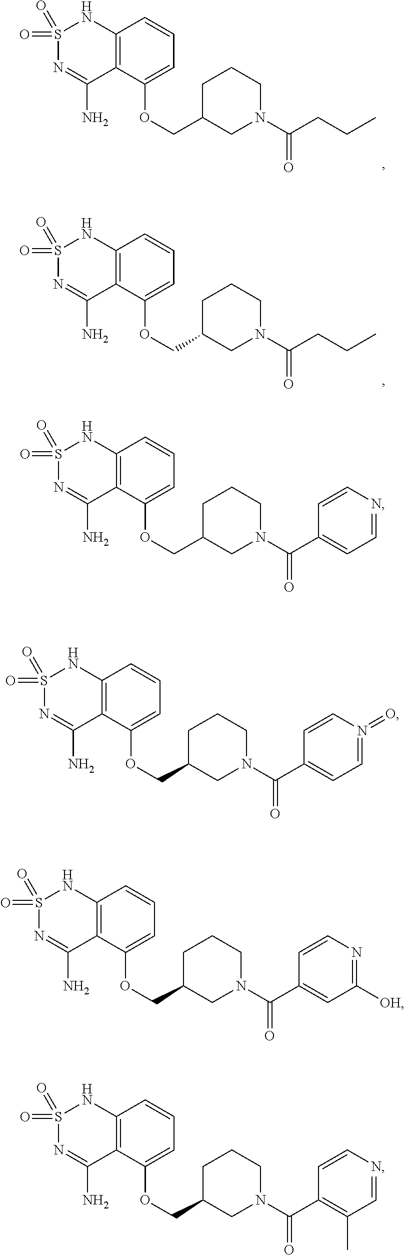 [SMILES: CCCC(=O)N1CCCC(COc2cccc3c2C(N)=NS(=O)(=O)N3)C1 ; CCCC(=O)N1CCC[C@@H](COc2cccc3c2C(N)=NS(=O)(=O)N3)C1 ; NC1=NS(=O)(=O)Nc2cccc(OCC3CCCN(C(=O)c4ccncc4)C3)c21 ; NC1=NS(=O)(=O)Nc2cccc(OC[C@H]3CCCN(C(=O)c4cc[n+]([O-])cc4)C3)c21 ; NC1=NS(=O)(=O)Nc2cccc(OC[C@H]3CCCN(C(=O)c4ccnc(O)c4)C3)c21 ; Cc1cnccc1C(=O)N1CCC[C@H](COc2cccc3c2C(N)=NS(=O)(=O)N3)C1]

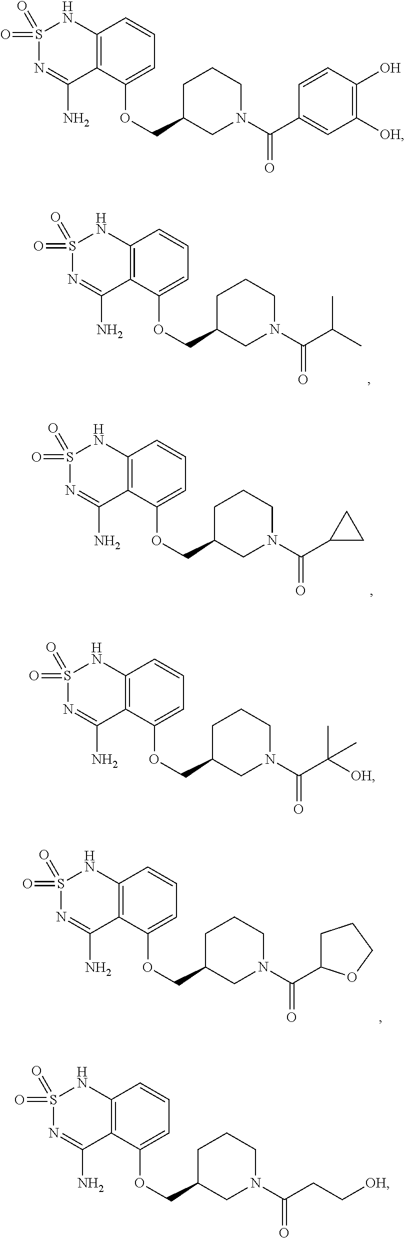

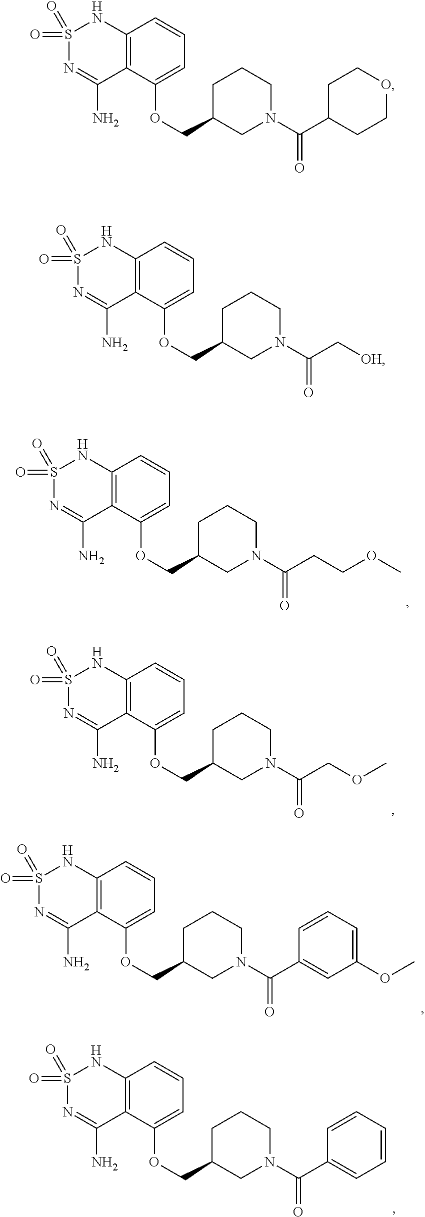 [SMILES: NC1=NS(=O)(=O)Nc2cccc(OC[C@H]3CCCN(C(=O)C4CCOCC4)C3)c21 ; NC1=NS(=O)(=O)Nc2cccc(OC[C@H]3CCCN(C(=O)CO)C3)c21 ; COCCC(=O)N1CCC[C@H](COc2cccc3c2C(N)=NS(=O)(=O)N3)C1 ; COCC(=O)N1CCC[C@H](COc2cccc3c2C(N)=NS(=O)(=O)N3)C1 ; COc1cccc(C(=O)N2CCC[C@H](COc3cccc4c3C(N)=NS(=O)(=O)N4)C2)c1 ; NC1=NS(=O)(=O)Nc2cccc(OC[C@H]3CCCN(C(=O)c4ccccc4)C3)c21]

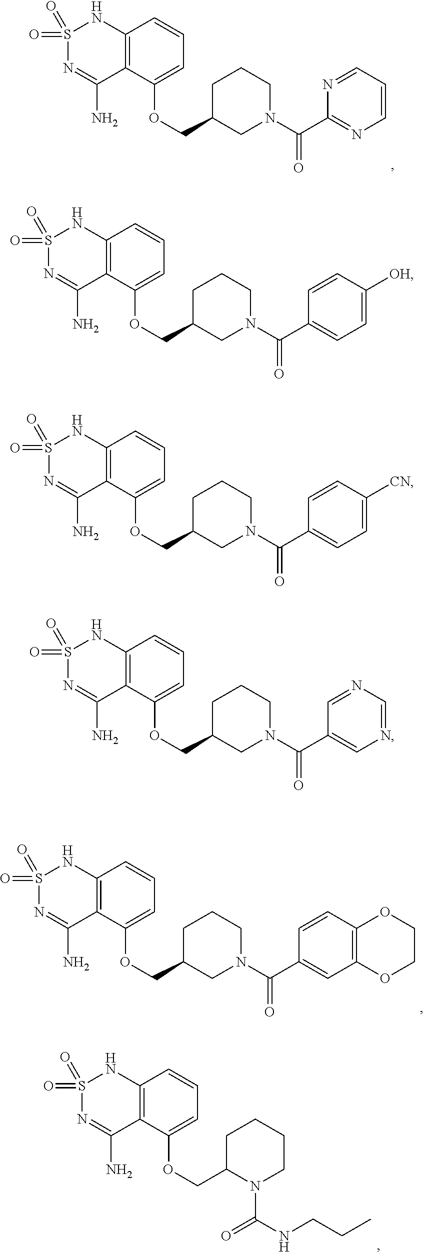

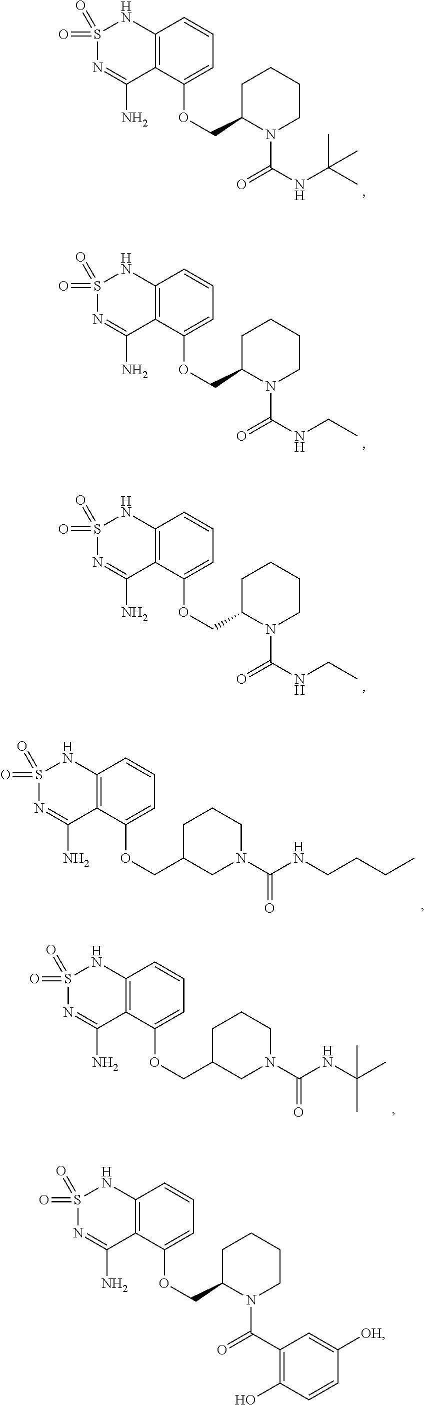 [SMILES: CC(C)(C)NC(=O)N1CCCC[C@@H]1COc1cccc2c1C(N)=NS(=O)(=O)N2 ; CCNC(=O)N1CCCC[C@@H]1COc1cccc2c1C(N)=NS(=O)(=O)N2 ; CCNC(=O)N1CCCC[C@H]1COc1cccc2c1C(N)=NS(=O)(=O)N2 ; CCCCNC(=O)N1CCCC(COc2cccc3c2C(N)=NS(=O)(=O)N3)C1 ; CC(C)(C)NC(=O)N1CCCC(COc2cccc3c2C(N)=NS(=O)(=O)N3)C1 ; NC1=NS(=O)(=O)Nc2cccc(OC[C@H]3CCCCN3C(=O)c3cc(O)ccc3O)c21]

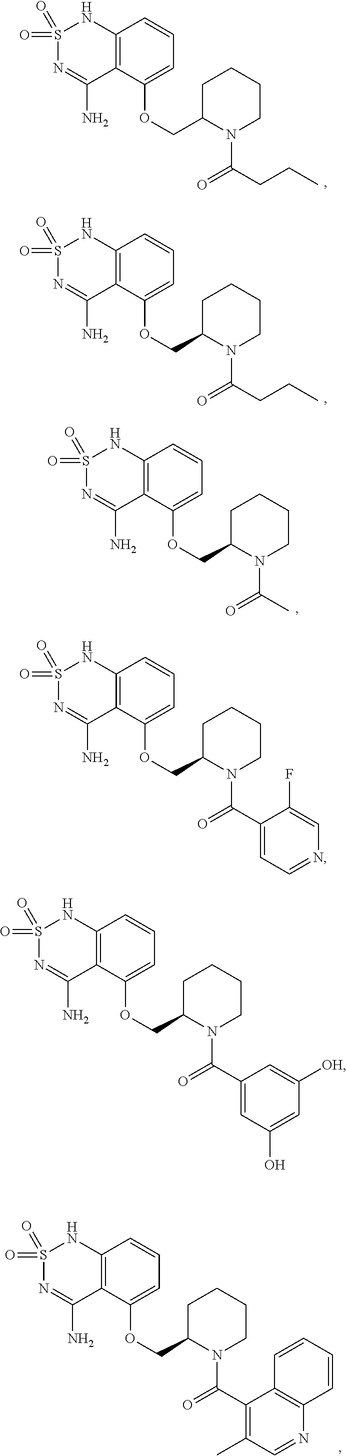 [SMILES: CCCC(=O)N1CCCCC1COc1cccc2c1C(N)=NS(=O)(=O)N2 ; CCCC(=O)N1CCCC[C@@H]1COc1cccc2c1C(N)=NS(=O)(=O)N2 ; CC(=O)N1CCCC[C@@H]1COc1cccc2c1C(N)=NS(=O)(=O)N2 ; NC1=NS(=O)(=O)Nc2cccc(OC[C@H]3CCCCN3C(=O)c3ccncc3F)c21 ; NC1=NS(=O)(=O)Nc2cccc(OC[C@H]3CCCCN3C(=O)c3cc(O)cc(O)c3)c21 ; Cc1cnc2ccccc2c1C(=O)N1CCCC[C@@H]1COc1cccc2c1C(N)=NS(=O)(=O)N2]

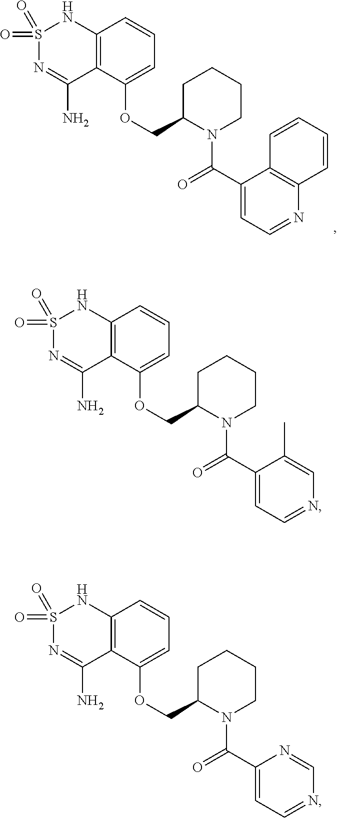 [SMILES: NC1=NS(=O)(=O)Nc2cccc(OC[C@H]3CCCCN3C(=O)c3ccnc4ccccc34)c21 ; Cc1cnccc1C(=O)N1CCCC[C@@H]1COc1cccc2c1C(N)=NS(=O)(=O)N2 ; NC1=NS(=O)(=O)Nc2cccc(OC[C@H]3CCCCN3C(=O)c3ccncn3)c21]

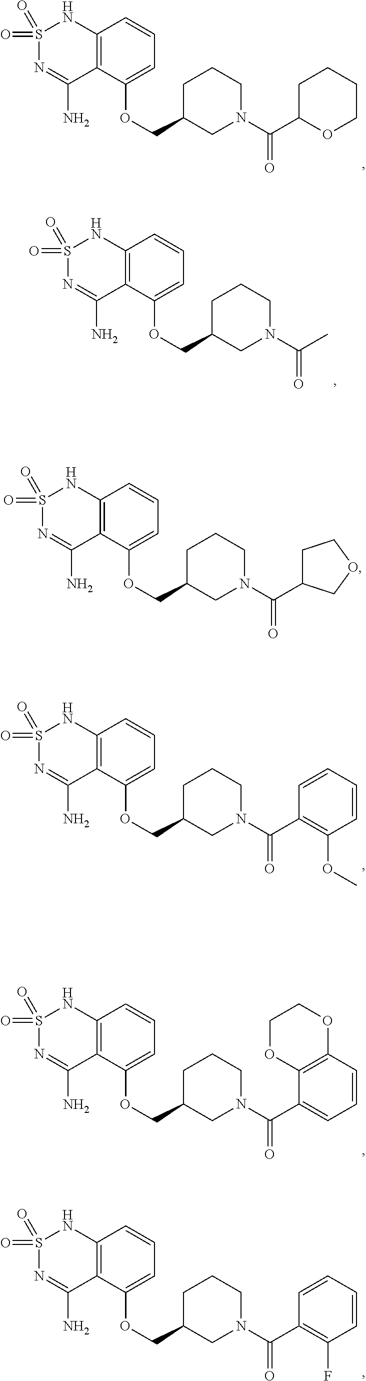 [SMILES: NC1=NS(=O)(=O)Nc2cccc(OC[C@H]3CCCN(C(=O)C4CCCCO4)C3)c21 ; CC(=O)N1CCC[C@H](COc2cccc3c2C(N)=NS(=O)(=O)N3)C1 ; NC1=NS(=O)(=O)Nc2cccc(OC[C@H]3CCCN(C(=O)C4CCOC4)C3)c21 ; COc1ccccc1C(=O)N1CCC[C@H](COc2cccc3c2C(N)=NS(=O)(=O)N3)C1 ; NC1=NS(=O)(=O)Nc2cccc(OC[C@H]3CCCN(C(=O)c4cccc5c4OCCO5)C3)c21 ; NC1=NS(=O)(=O)Nc2cccc(OC[C@H]3CCCN(C(=O)c4ccccc4F)C3)c21]

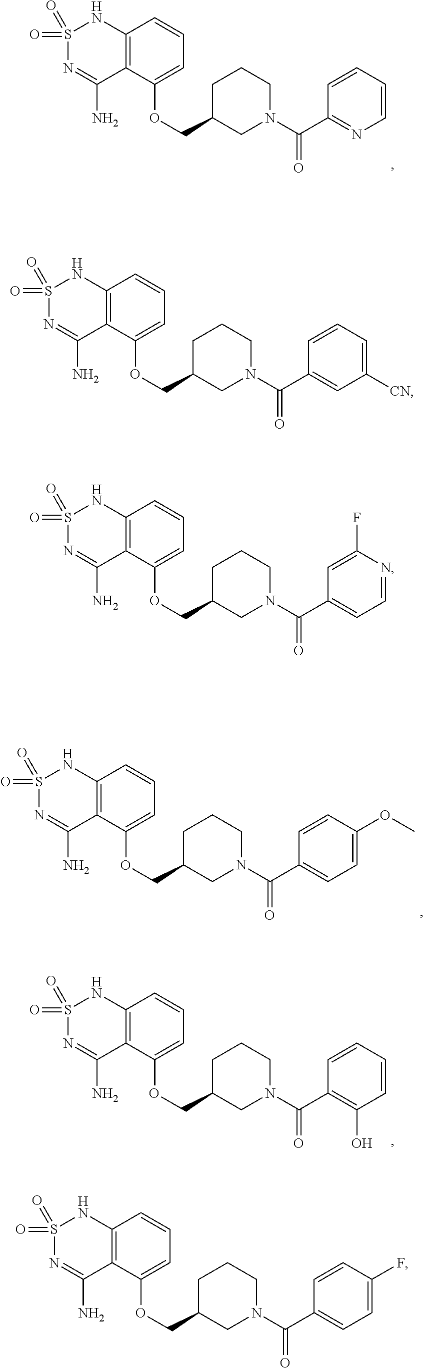 [SMILES: NC1=NS(=O)(=O)Nc2cccc(OC[C@H]3CCCN(C(=O)c4ccccn4)C3)c21 ; N#Cc1cccc(C(=O)N2CCC[C@H](COc3cccc4c3C(N)=NS(=O)(=O)N4)C2)c1 ; NC1=NS(=O)(=O)Nc2cccc(OC[C@H]3CCCN(C(=O)c4ccnc(F)c4)C3)c21 ; COc1ccc(C(=O)N2CCC[C@H](COc3cccc4c3C(N)=NS(=O)(=O)N4)C2)cc1 ; NC1=NS(=O)(=O)Nc2cccc(OC[C@H]3CCCN(C(=O)c4ccccc4O)C3)c21 ; NC1=NS(=O)(=O)Nc2cccc(OC[C@H]3CCCN(C(=O)c4ccc(F)cc4)C3)c21]

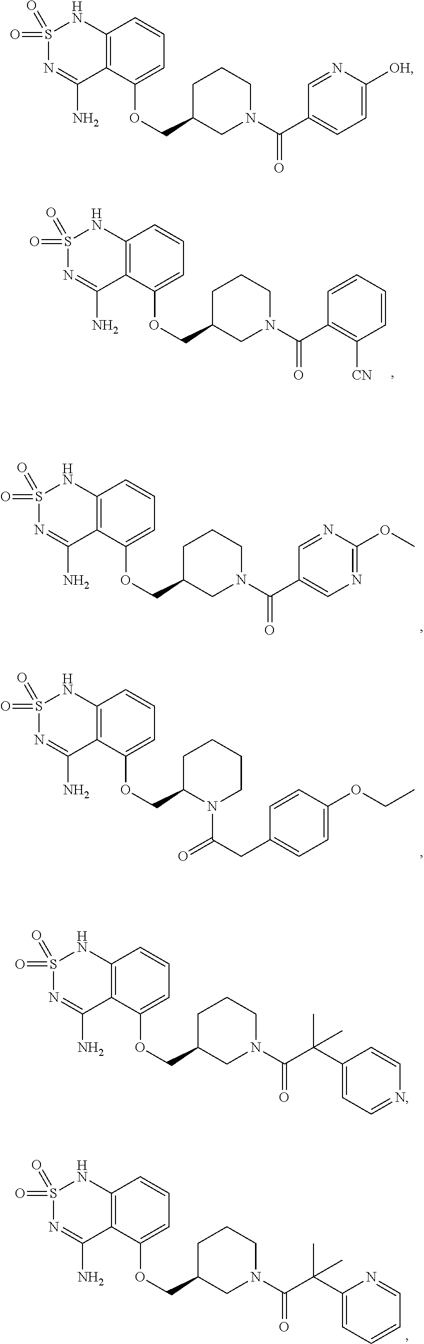 [SMILES: NC1=NS(=O)(=O)Nc2cccc(OC[C@H]3CCCN(C(=O)c4ccc(O)nc4)C3)c21 ; N#Cc1ccccc1C(=O)N1CCC[C@H](COc2cccc3c2C(N)=NS(=O)(=O)N3)C1 ; COc1ncc(C(=O)N2CCC[C@H](COc3cccc4c3C(N)=NS(=O)(=O)N4)C2)cn1 ; CCOc1ccc(CC(=O)N2CCCC[C@@H]2COc2cccc3c2C(N)=NS(=O)(=O)N3)cc1 ; CC(C)(C(=O)N1CCC[C@H](COc2cccc3c2C(N)=NS(=O)(=O)N3)C1)c1ccncc1 ; CC(C)(C(=O)N1CCC[C@H](COc2cccc3c2C(N)=NS(=O)(=O)N3)C1)c1ccccn1]

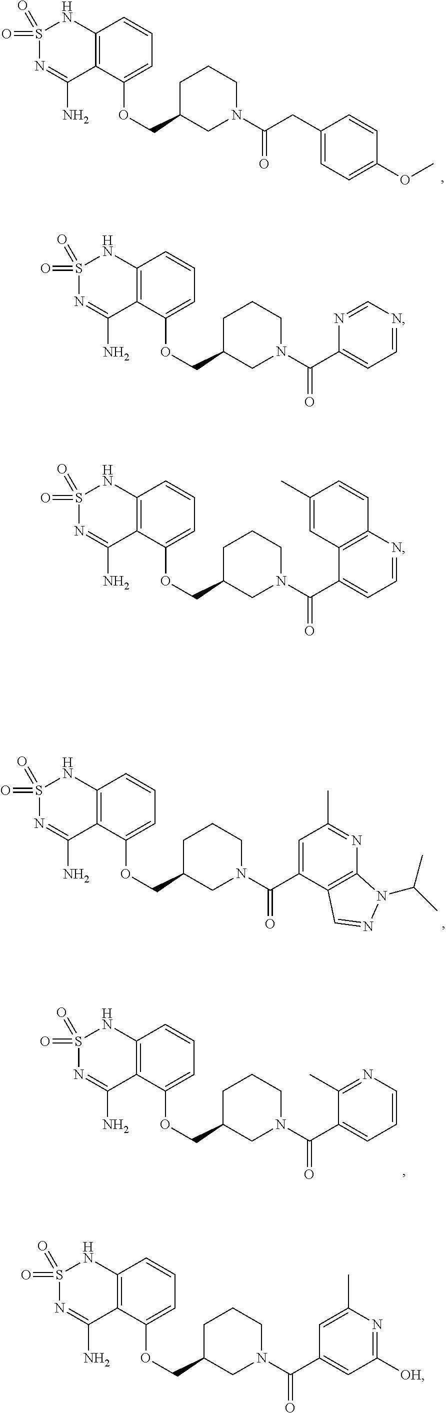 [SMILES: COc1ccc(CC(=O)N2CCC[C@H](COc3cccc4c3C(N)=NS(=O)(=O)N4)C2)cc1 ; NC1=NS(=O)(=O)Nc2cccc(OC[C@H]3CCCN(C(=O)c4ccncn4)C3)c21 ; Cc1ccc2nccc(C(=O)N3CCC[C@H](COc4cccc5c4C(N)=NS(=O)(=O)N5)C3)c2c1 ; Cc1cc(C(=O)N2CCC[C@H](COc3cccc4c3C(N)=NS(=O)(=O)N4)C2)c2cnn(C(C)C)c2n1 ; Cc1ncccc1C(=O)N1CCC[C@H](COc2cccc3c2C(N)=NS(=O)(=O)N3)C1 ; Cc1cc(C(=O)N2CCC[C@H](COc3cccc4c3C(N)=NS(=O)(=O)N4)C2)cc(O)n1]

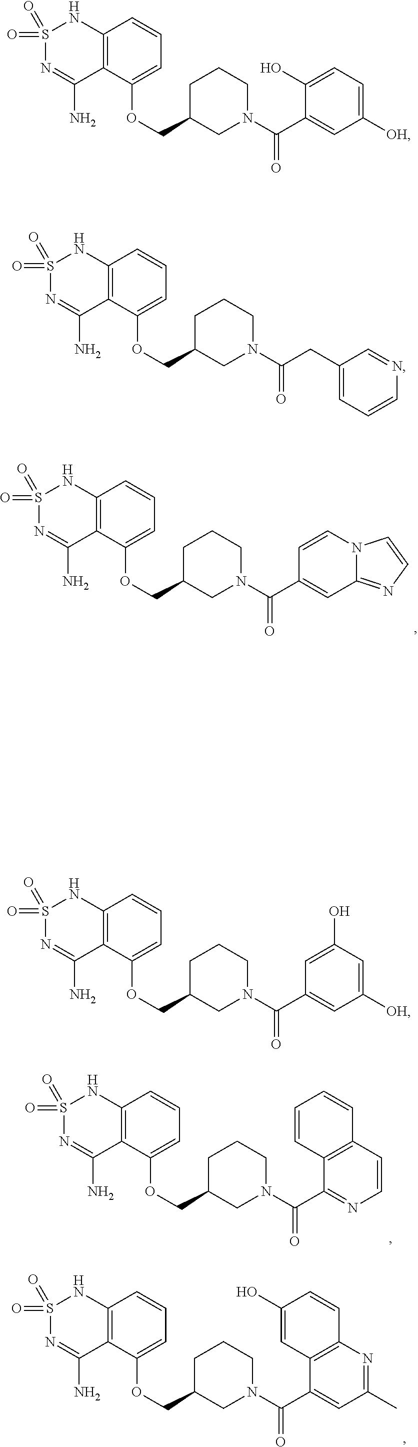 [SMILES: NC1=NS(=O)(=O)Nc2cccc(OC[C@H]3CCCN(C(=O)c4cc(O)ccc4O)C3)c21 ; NC1=NS(=O)(=O)Nc2cccc(OC[C@H]3CCCN(C(=O)Cc4cccnc4)C3)c21 ; NC1=NS(=O)(=O)Nc2cccc(OC[C@H]3CCCN(C(=O)c4ccn5ccnc5c4)C3)c21 ; NC1=NS(=O)(=O)Nc2cccc(OC[C@H]3CCCN(C(=O)c4cc(O)cc(O)c4)C3)c21 ; NC1=NS(=O)(=O)Nc2cccc(OC[C@H]3CCCN(C(=O)c4nccc5ccccc45)C3)c21 ; Cc1cc(C(=O)N2CCC[C@H](COc3cccc4c3C(N)=NS(=O)(=O)N4)C2)c2cc(O)ccc2n1]

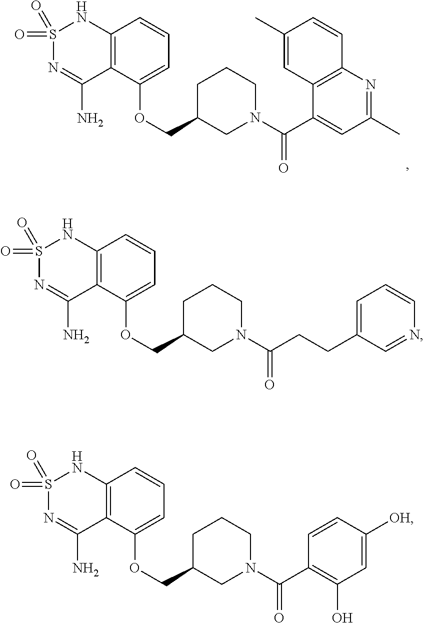 [SMILES: Cc1ccc2nc(C)cc(C(=O)N3CCC[C@H](COc4cccc5c4C(N)=NS(=O)(=O)N5)C3)c2c1 ; NC1=NS(=O)(=O)Nc2cccc(OC[C@H]3CCCN(C(=O)CCc4cccnc4)C3)c21 ; NC1=NS(=O)(=O)Nc2cccc(OC[C@H]3CCCN(C(=O)c4ccc(O)cc4O)C3)c21]

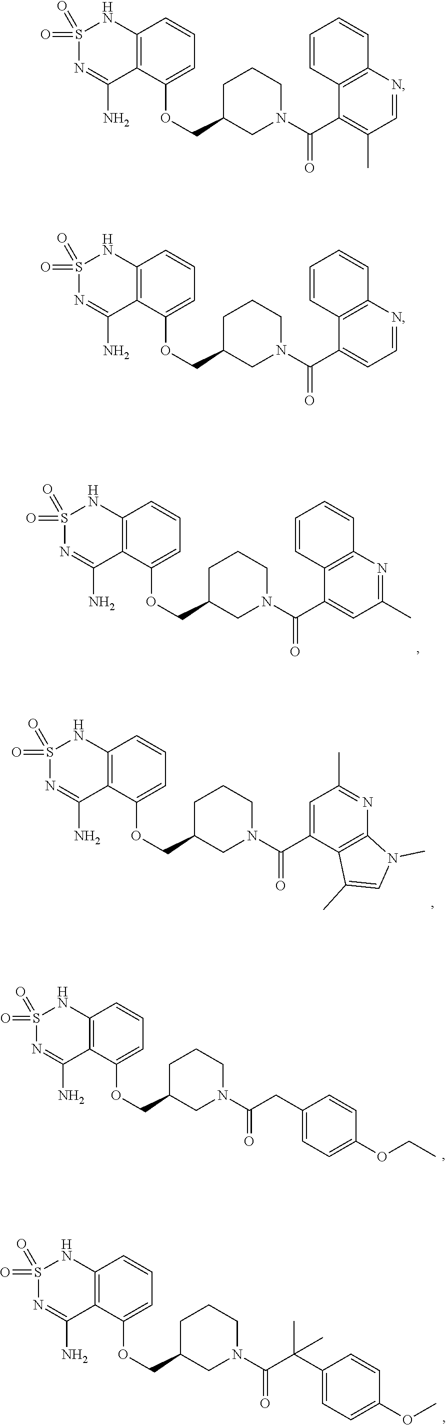 [SMILES: Cc1cnc2ccccc2c1C(=O)N1CCC[C@H](COc2cccc3c2C(N)=NS(=O)(=O)N3)C1 ; NC1=NS(=O)(=O)Nc2cccc(OC[C@H]3CCCN(C(=O)c4ccnc5ccccc45)C3)c21 ; Cc1cc(C(=O)N2CCC[C@H](COc3cccc4c3C(N)=NS(=O)(=O)N4)C2)c2ccccc2n1 ; Cc1cc(C(=O)N2CCC[C@H](COc3cccc4c3C(N)=NS(=O)(=O)N4)C2)c2c(C)cn(C)c2n1 ; CCOc1ccc(CC(=O)N2CCC[C@H](COc3cccc4c3C(N)=NS(=O)(=O)N4)C2)cc1 ; COc1ccc(C(C)(C)C(=O)N2CCC[C@H](COc3cccc4c3C(N)=NS(=O)(=O)N4)C2)cc1]

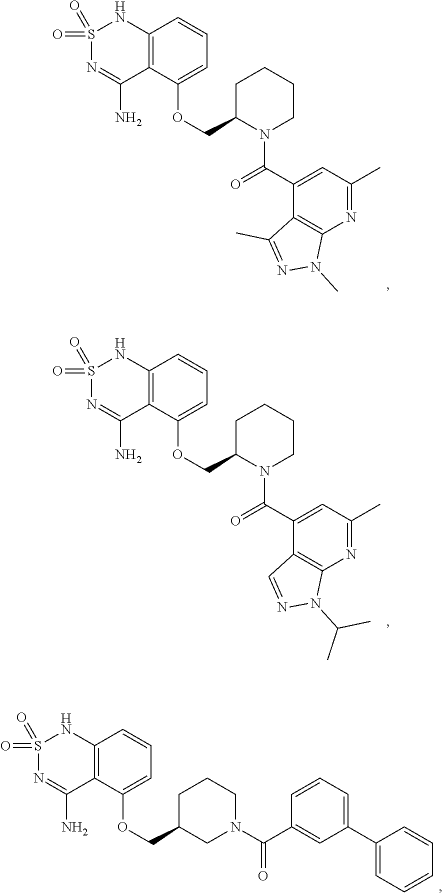 [SMILES: Cc1cc(C(=O)N2CCCC[C@@H]2COc2cccc3c2C(N)=NS(=O)(=O)N3)c2c(C)nn(C)c2n1 ; Cc1cc(C(=O)N2CCCC[C@@H]2COc2cccc3c2C(N)=NS(=O)(=O)N3)c2cnn(C(C)C)c2n1 ; NC1=NS(=O)(=O)Nc2cccc(OC[C@H]3CCCN(C(=O)c4cccc(-c5ccccc5)c4)C3)c21]

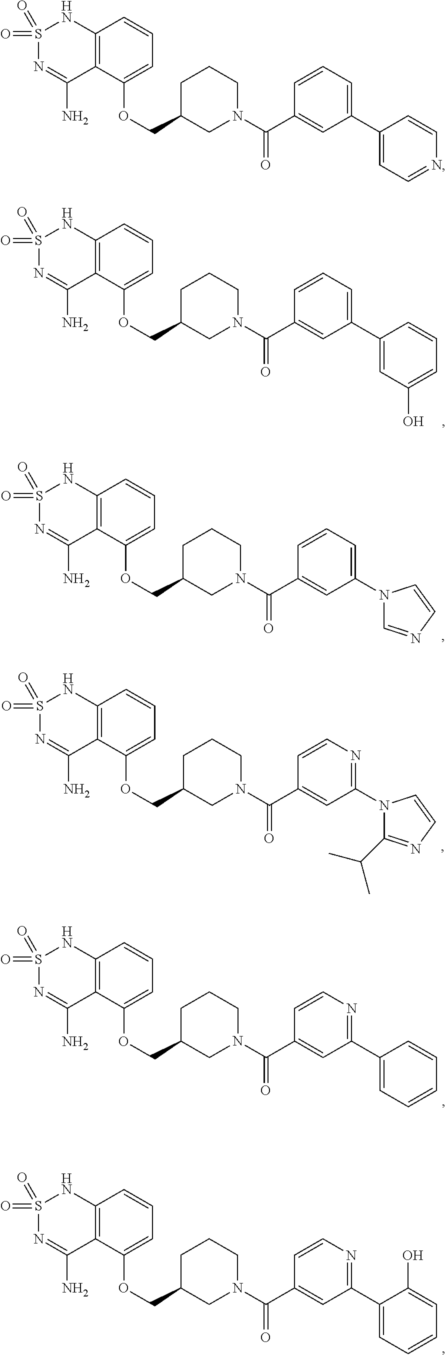

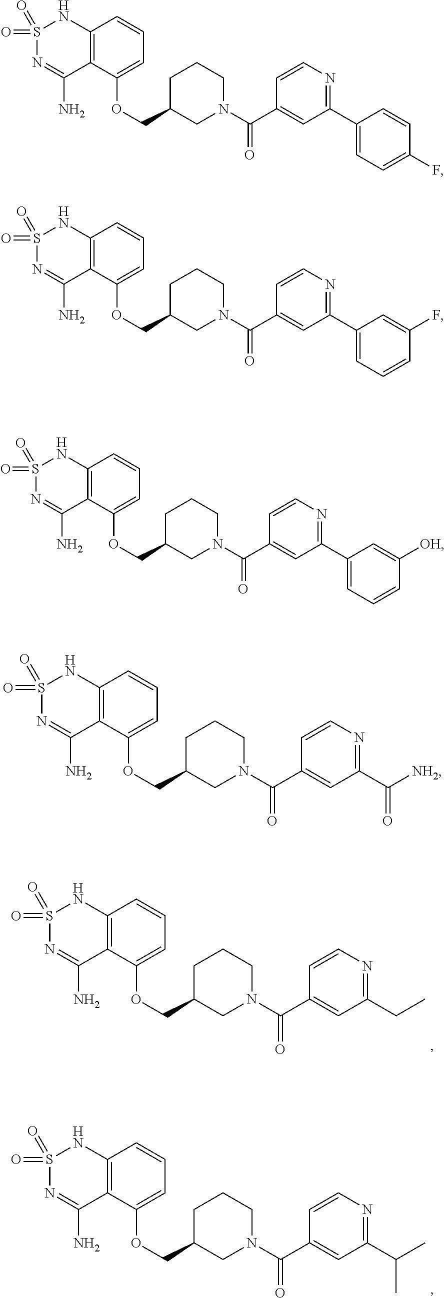 [SMILES: NC1=NS(=O)(=O)Nc2cccc(OC[C@H]3CCCN(C(=O)c4ccnc(-c5ccc(F)cc5)c4)C3)c21 ; NC1=NS(=O)(=O)Nc2cccc(OC[C@H]3CCCN(C(=O)c4ccnc(-c5cccc(F)c5)c4)C3)c21 ; NC1=NS(=O)(=O)Nc2cccc(OC[C@H]3CCCN(C(=O)c4ccnc(-c5cccc(O)c5)c4)C3)c21 ; NC(=O)c1cc(C(=O)N2CCC[C@H](COc3cccc4c3C(N)=NS(=O)(=O)N4)C2)ccn1 ; CCc1cc(C(=O)N2CCC[C@H](COc3cccc4c3C(N)=NS(=O)(=O)N4)C2)ccn1 ; CC(C)c1cc(C(=O)N2CCC[C@H](COc3cccc4c3C(N)=NS(=O)(=O)N4)C2)ccn1]

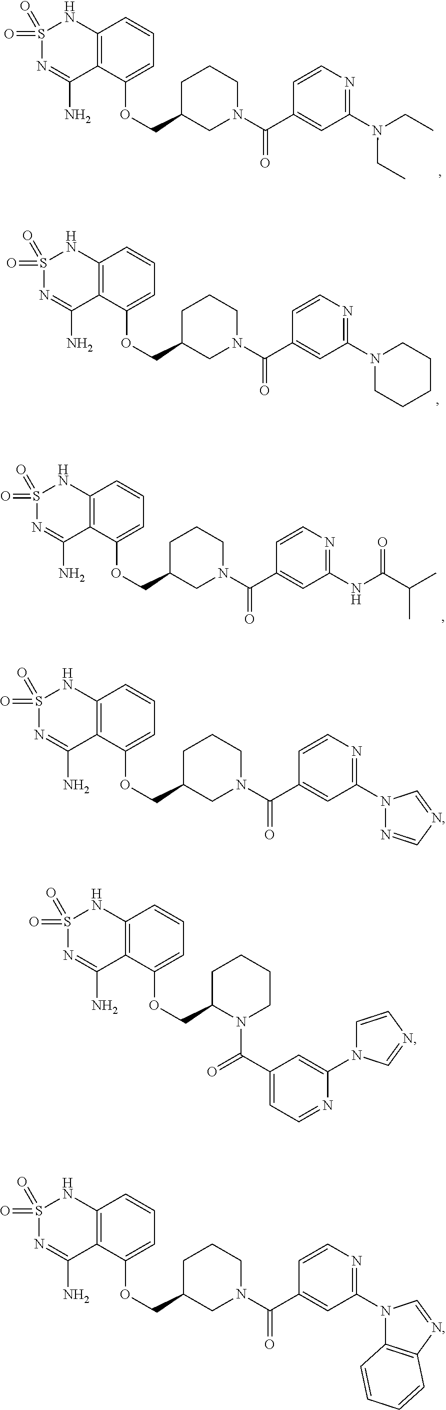 [SMILES: CCN(CC)c1cc(C(=O)N2CCC[C@H](COc3cccc4c3C(N)=NS(=O)(=O)N4)C2)ccn1 ; NC1=NS(=O)(=O)Nc2cccc(OC[C@H]3CCCN(C(=O)c4ccnc(N5CCCCC5)c4)C3)c21 ; CC(C)C(=O)Nc1cc(C(=O)N2CCC[C@H](COc3cccc4c3C(N)=NS(=O)(=O)N4)C2)ccn1 ; NC1=NS(=O)(=O)Nc2cccc(OC[C@H]3CCCN(C(=O)c4ccnc(-n5cncn5)c4)C3)c21 ; NC1=NS(=O)(=O)Nc2cccc(OC[C@H]3CCCCN3C(=O)c3ccnc(-n4ccnc4)c3)c21 ; NC1=NS(=O)(=O)Nc2cccc(OC[C@H]3CCCN(C(=O)c4ccnc(-n5cnc6ccccc65)c4)C3)c21]

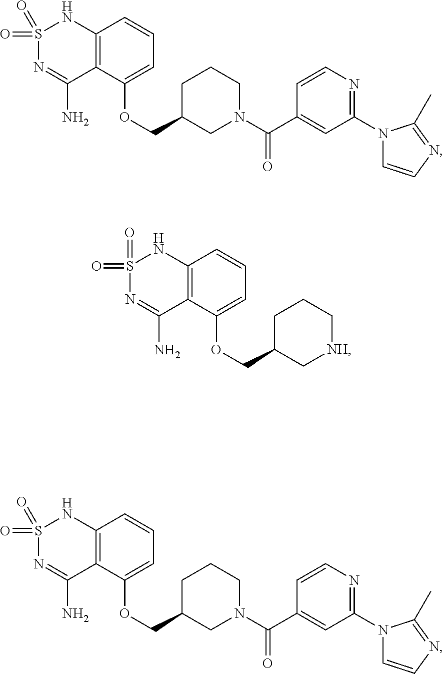 [SMILES: Cc1nccn1-c1cc(C(=O)N2CCC[C@H](COc3cccc4c3C(N)=NS(=O)(=O)N4)C2)ccn1 ; NC1=NS(=O)(=O)Nc2cccc(OC[C@H]3CCCNC3)c21 ; Cc1nccn1-c1cc(C(=O)N2CCC[C@H](COc3cccc4c3C(N)=NS(=O)(=O)N4)C2)ccn1]

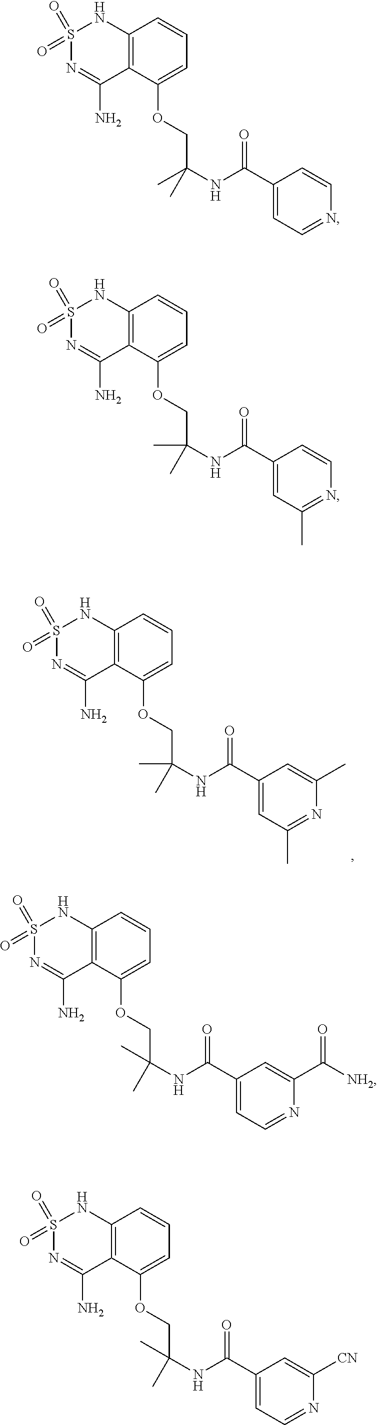 [SMILES: CC(C)(COc1cccc2c1C(N)=NS(=O)(=O)N2)NC(=O)c1ccncc1 ; Cc1cc(C(=O)NC(C)(C)COc2cccc3c2C(N)=NS(=O)(=O)N3)ccn1 ; Cc1cc(C(=O)NC(C)(C)COc2cccc3c2C(N)=NS(=O)(=O)N3)cc(C)n1 ; CC(C)(COc1cccc2c1C(N)=NS(=O)(=O)N2)NC(=O)c1ccnc(C(N)=O)c1 ; CC(C)(COc1cccc2c1C(N)=NS(=O)(=O)N2)NC(=O)c1ccnc(C#N)c1]

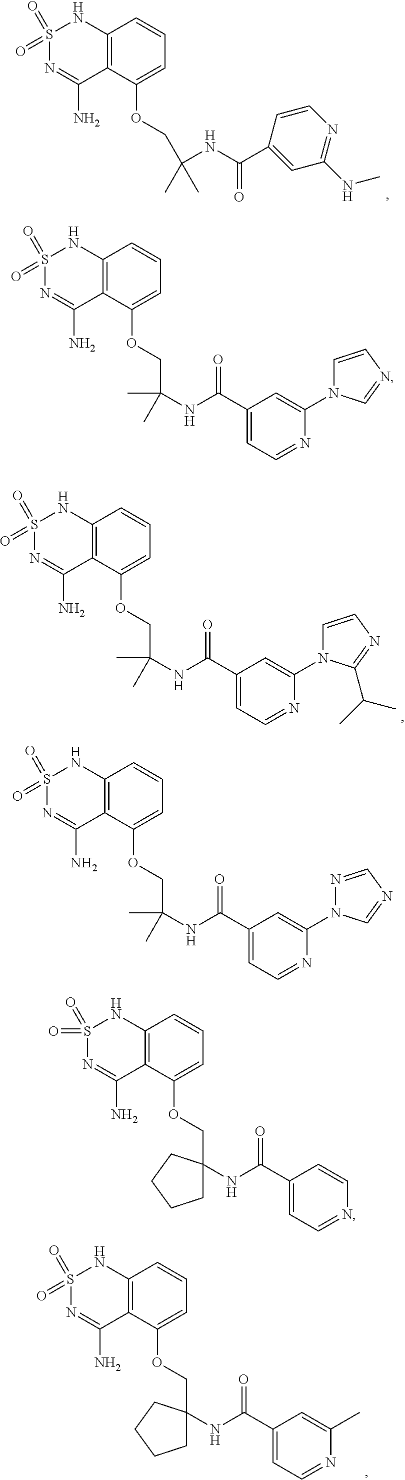 [SMILES: CNc1cc(C(=O)NC(C)(C)COc2cccc3c2C(N)=NS(=O)(=O)N3)ccn1 ; CC(C)(COc1cccc2c1C(N)=NS(=O)(=O)N2)NC(=O)c1ccnc(-n2ccnc2)c1 ; CC(C)c1nccn1-c1cc(C(=O)NC(C)(C)COc2cccc3c2C(N)=NS(=O)(=O)N3)ccn1 ; CC(C)(COc1cccc2c1C(N)=NS(=O)(=O)N2)NC(=O)c1ccnc(-n2cncn2)c1 ; NC1=NS(=O)(=O)Nc2cccc(OCC3(NC(=O)c4ccncc4)CCCC3)c21 ; Cc1cc(C(=O)NC2(COc3cccc4c3C(N)=NS(=O)(=O)N4)CCCC2)ccn1]

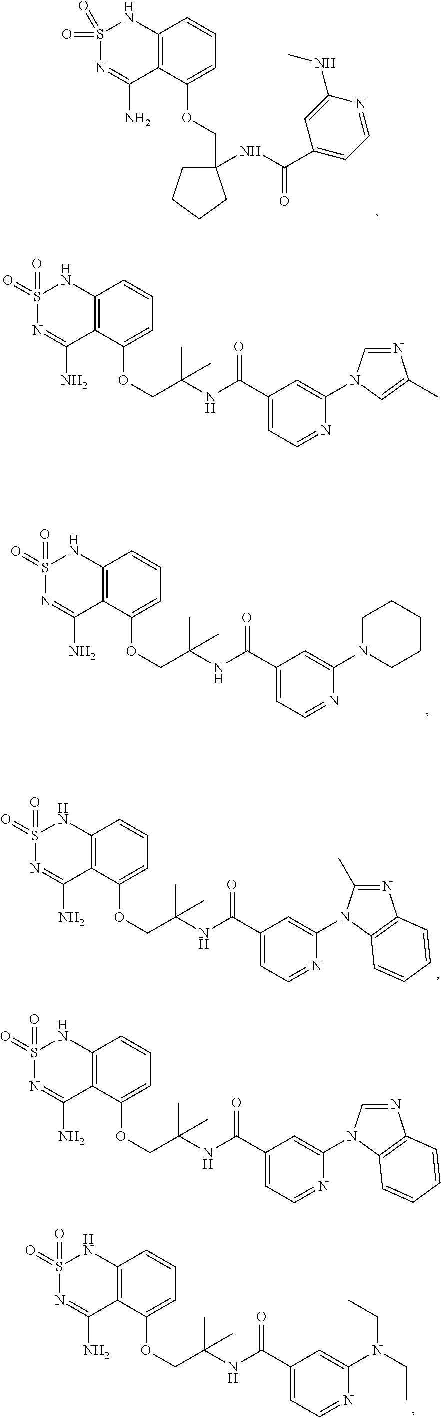 [SMILES: CNc1cc(C(=O)NC2(COc3cccc4c3C(N)=NS(=O)(=O)N4)CCCC2)ccn1 ; Cc1cn(-c2cc(C(=O)NC(C)(C)COc3cccc4c3C(N)=NS(=O)(=O)N4)ccn2)cn1 ; CC(C)(COc1cccc2c1C(N)=NS(=O)(=O)N2)NC(=O)c1ccnc(N2CCCCC2)c1 ; Cc1nc2ccccc2n1-c1cc(C(=O)NC(C)(C)COc2cccc3c2C(N)=NS(=O)(=O)N3)ccn1 ; CC(C)(COc1cccc2c1C(N)=NS(=O)(=O)N2)NC(=O)c1ccnc(-n2cnc3ccccc32)c1 ; CCN(CC)c1cc(C(=O)NC(C)(C)COc2cccc3c2C(N)=NS(=O)(=O)N3)ccn1]

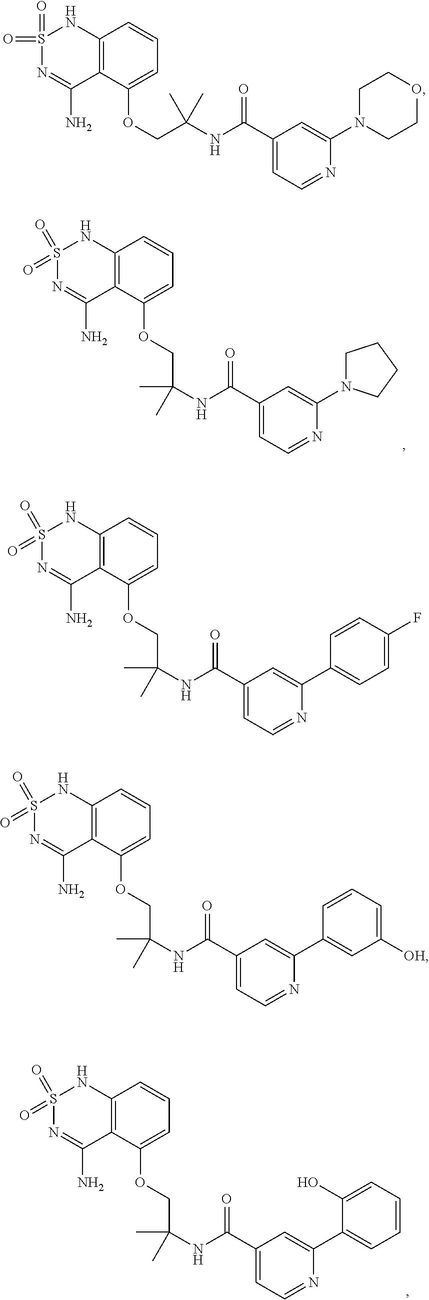 [SMILES: CC(C)(COc1cccc2c1C(N)=NS(=O)(=O)N2)NC(=O)c1ccnc(N2CCOCC2)c1 ; CC(C)(COc1cccc2c1C(N)=NS(=O)(=O)N2)NC(=O)c1ccnc(N2CCCC2)c1 ; CC(C)(COc1cccc2c1C(N)=NS(=O)(=O)N2)NC(=O)c1ccnc(-c2ccc(F)cc2)c1 ; CC(C)(COc1cccc2c1C(N)=NS(=O)(=O)N2)NC(=O)c1ccnc(-c2cccc(O)c2)c1 ; CC(C)(COc1cccc2c1C(N)=NS(=O)(=O)N2)NC(=O)c1ccnc(-c2ccccc2O)c1]

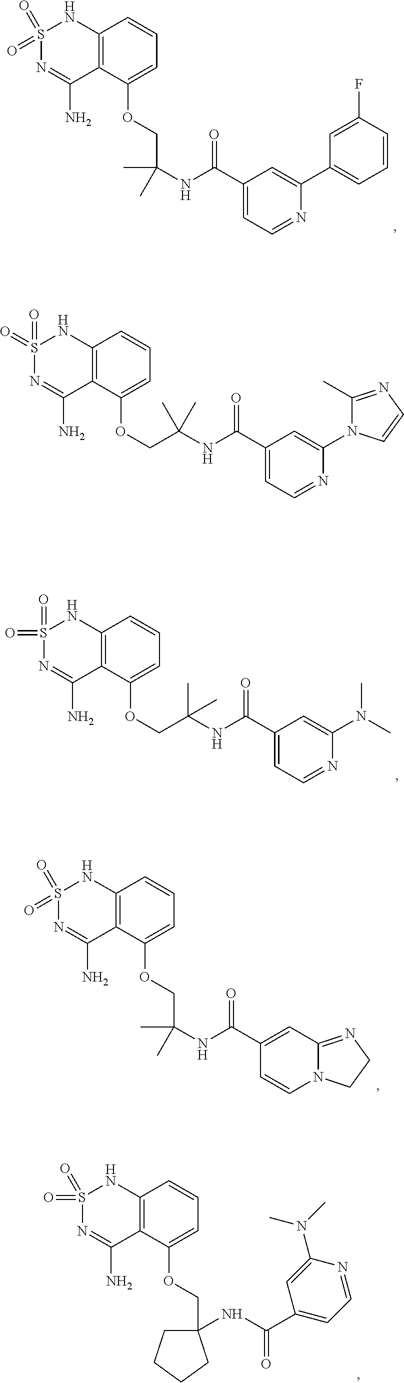 [SMILES: CC(C)(COc1cccc2c1C(N)=NS(=O)(=O)N2)NC(=O)c1ccnc(-c2cccc(F)c2)c1 ; Cc1nccn1-c1cc(C(=O)NC(C)(C)COc2cccc3c2C(N)=NS(=O)(=O)N3)ccn1 ; CN(C)c1cc(C(=O)NC(C)(C)COc2cccc3c2C(N)=NS(=O)(=O)N3)ccn1 ; CC(C)(COc1cccc2c1C(N)=NS(=O)(=O)N2)NC(=O)C1=CC2=NCCN2C=C1 ; CN(C)c1cc(C(=O)NC2(COc3cccc4c3C(N)=NS(=O)(=O)N4)CCCC2)ccn1]

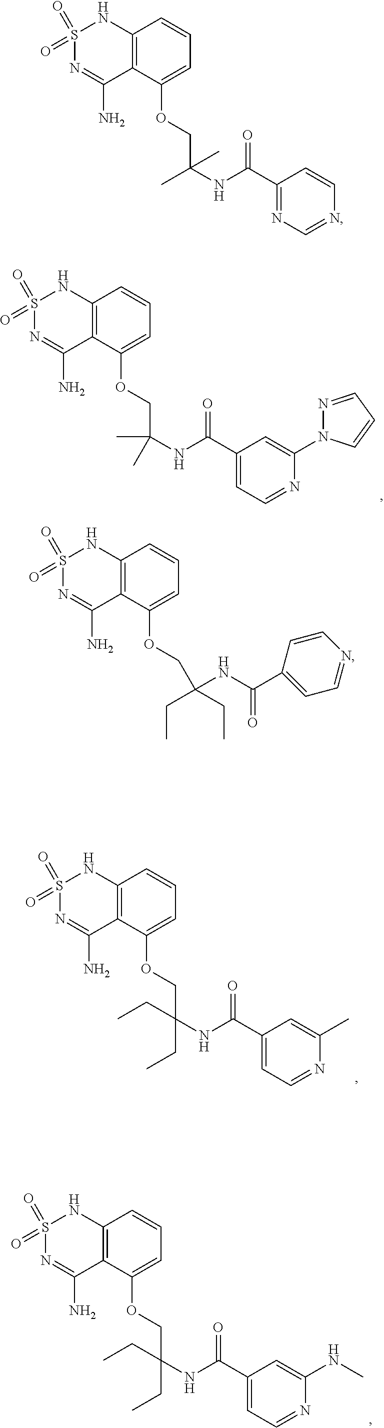 [SMILES: CC(C)(COc1cccc2c1C(N)=NS(=O)(=O)N2)NC(=O)c1ccncn1 ; CC(C)(COc1cccc2c1C(N)=NS(=O)(=O)N2)NC(=O)c1ccnc(-n2cccn2)c1 ; CCC(CC)(COc1cccc2c1C(N)=NS(=O)(=O)N2)NC(=O)c1ccncc1 ; CCC(CC)(COc1cccc2c1C(N)=NS(=O)(=O)N2)NC(=O)c1ccnc(C)c1 ; CCC(CC)(COc1cccc2c1C(N)=NS(=O)(=O)N2)NC(=O)c1ccnc(NC)c1]

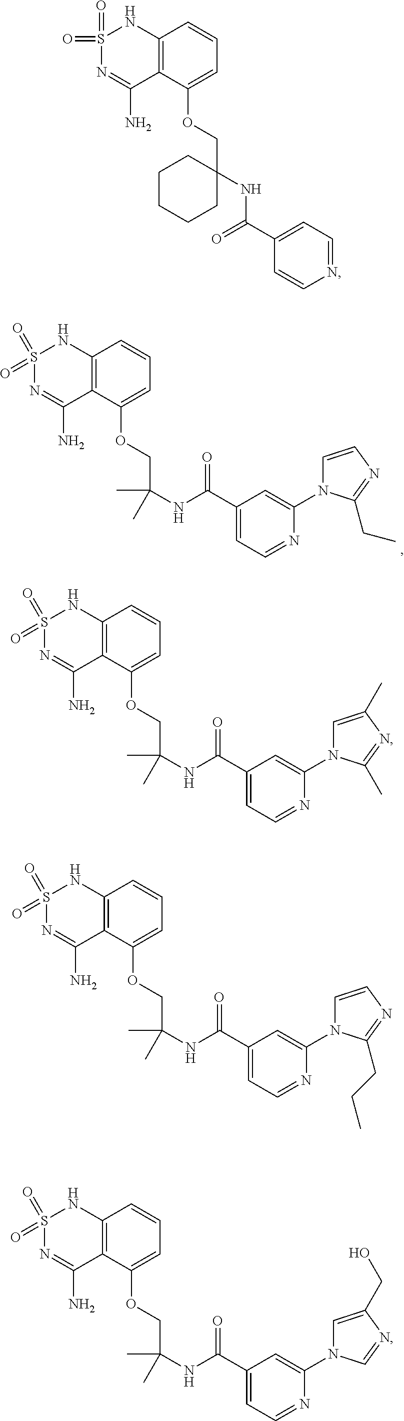 [SMILES: NC1=NS(=O)(=O)Nc2cccc(OCC3(NC(=O)c4ccncc4)CCCCC3)c21 ; CCc1nccn1-c1cc(C(=O)NC(C)(C)COc2cccc3c2C(N)=NS(=O)(=O)N3)ccn1 ; Cc1cn(-c2cc(C(=O)NC(C)(C)COc3cccc4c3C(N)=NS(=O)(=O)N4)ccn2)c(C)n1 ; CCCc1nccn1-c1cc(C(=O)NC(C)(C)COc2cccc3c2C(N)=NS(=O)(=O)N3)ccn1 ; CC(C)(COc1cccc2c1C(N)=NS(=O)(=O)N2)NC(=O)c1ccnc(-n2cnc(CO)c2)c1]

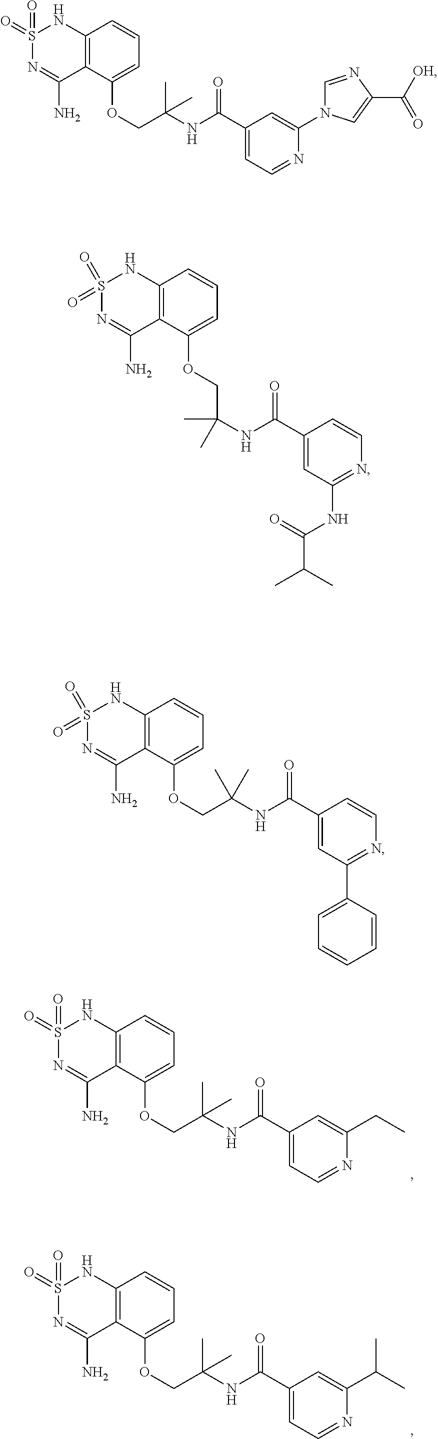 [SMILES: CC(C)(COc1cccc2c1C(N)=NS(=O)(=O)N2)NC(=O)c1ccnc(-n2cnc(C(=O)O)c2)c1 ; CC(C)C(=O)Nc1cc(C(=O)NC(C)(C)COc2cccc3c2C(N)=NS(=O)(=O)N3)ccn1 ; CC(C)(COc1cccc2c1C(N)=NS(=O)(=O)N2)NC(=O)c1ccnc(-c2ccccc2)c1 ; CCc1cc(C(=O)NC(C)(C)COc2cccc3c2C(N)=NS(=O)(=O)N3)ccn1 ; CC(C)c1cc(C(=O)NC(C)(C)COc2cccc3c2C(N)=NS(=O)(=O)N3)ccn1]

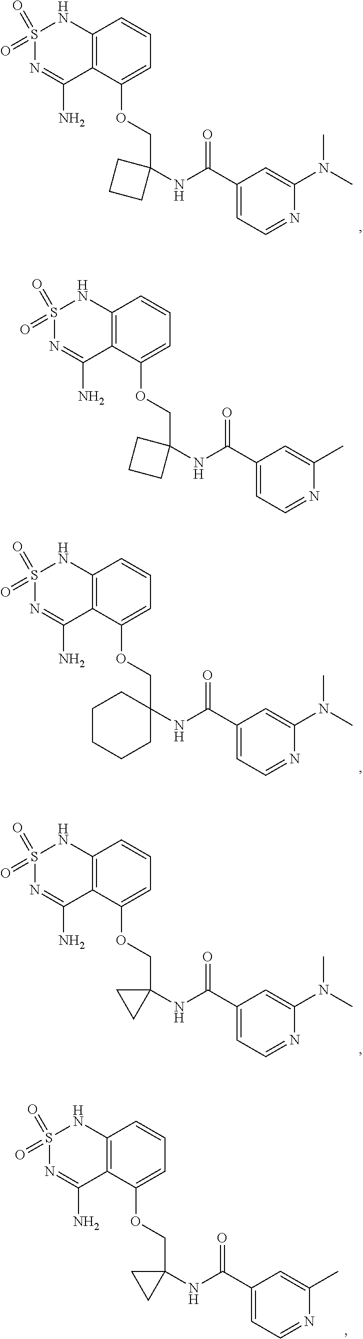 [SMILES: CN(C)c1cc(C(=O)NC2(COc3cccc4c3C(N)=NS(=O)(=O)N4)CCC2)ccn1 ; Cc1cc(C(=O)NC2(COc3cccc4c3C(N)=NS(=O)(=O)N4)CCC2)ccn1 ; CN(C)c1cc(C(=O)NC2(COc3cccc4c3C(N)=NS(=O)(=O)N4)CCCCC2)ccn1 ; CN(C)c1cc(C(=O)NC2(COc3cccc4c3C(N)=NS(=O)(=O)N4)CC2)ccn1 ; Cc1cc(C(=O)NC2(COc3cccc4c3C(N)=NS(=O)(=O)N4)CC2)ccn1]

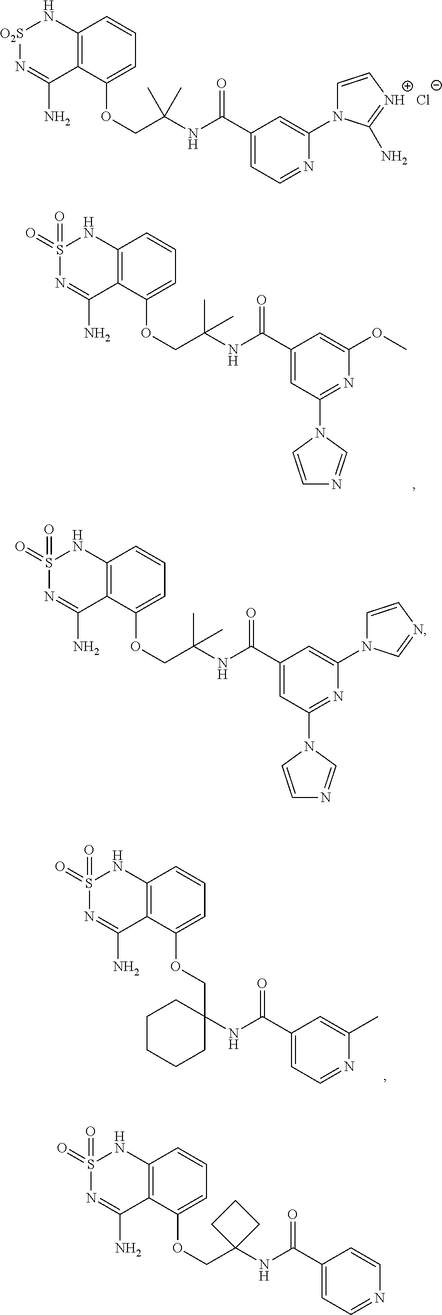

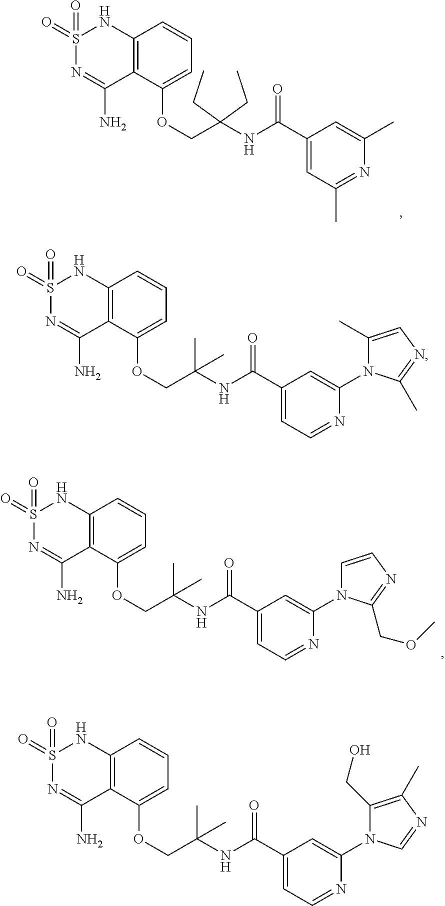 [SMILES: CCC(CC)(COc1cccc2c1C(N)=NS(=O)(=O)N2)NC(=O)c1cc(C)nc(C)c1 ; Cc1cnc(C)n1-c1cc(C(=O)NC(C)(C)COc2cccc3c2C(N)=NS(=O)(=O)N3)ccn1 ; COCc1nccn1-c1cc(C(=O)NC(C)(C)COc2cccc3c2C(N)=NS(=O)(=O)N3)ccn1 ; Cc1ncn(-c2cc(C(=O)NC(C)(C)COc3cccc4c3C(N)=NS(=O)(=O)N4)ccn2)c1CO]

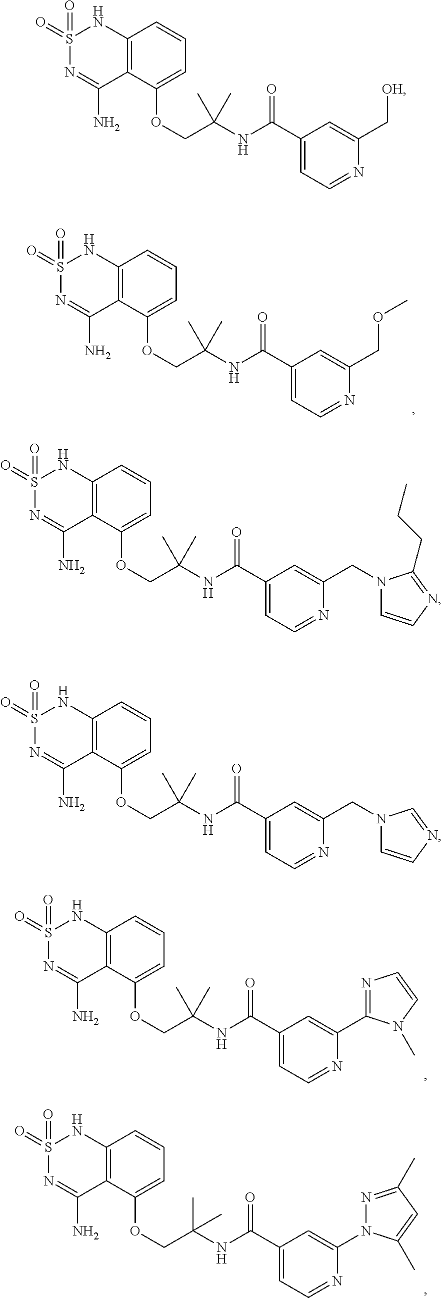 [SMILES: CC(C)(COc1cccc2c1C(N)=NS(=O)(=O)N2)NC(=O)c1ccnc(CO)c1 ; COCc1cc(C(=O)NC(C)(C)COc2cccc3c2C(N)=NS(=O)(=O)N3)ccn1 ; CCCc1nccn1Cc1cc(C(=O)NC(C)(C)COc2cccc3c2C(N)=NS(=O)(=O)N3)ccn1 ; CC(C)(COc1cccc2c1C(N)=NS(=O)(=O)N2)NC(=O)c1ccnc(Cn2ccnc2)c1 ; Cn1ccnc1-c1cc(C(=O)NC(C)(C)COc2cccc3c2C(N)=NS(=O)(=O)N3)ccn1 ; Cc1cc(C)n(-c2cc(C(=O)NC(C)(C)COc3cccc4c3C(N)=NS(=O)(=O)N4)ccn2)n1]

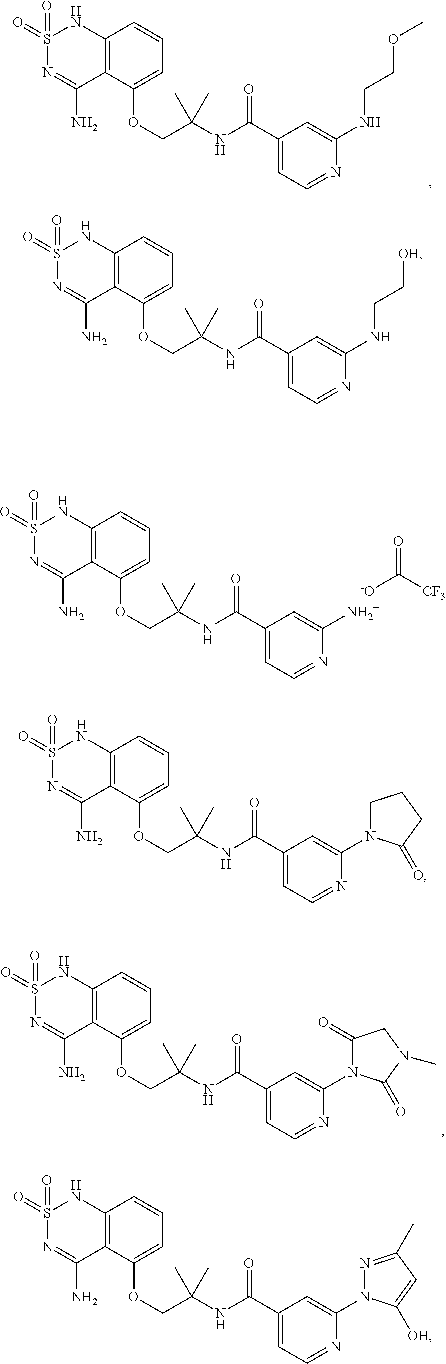

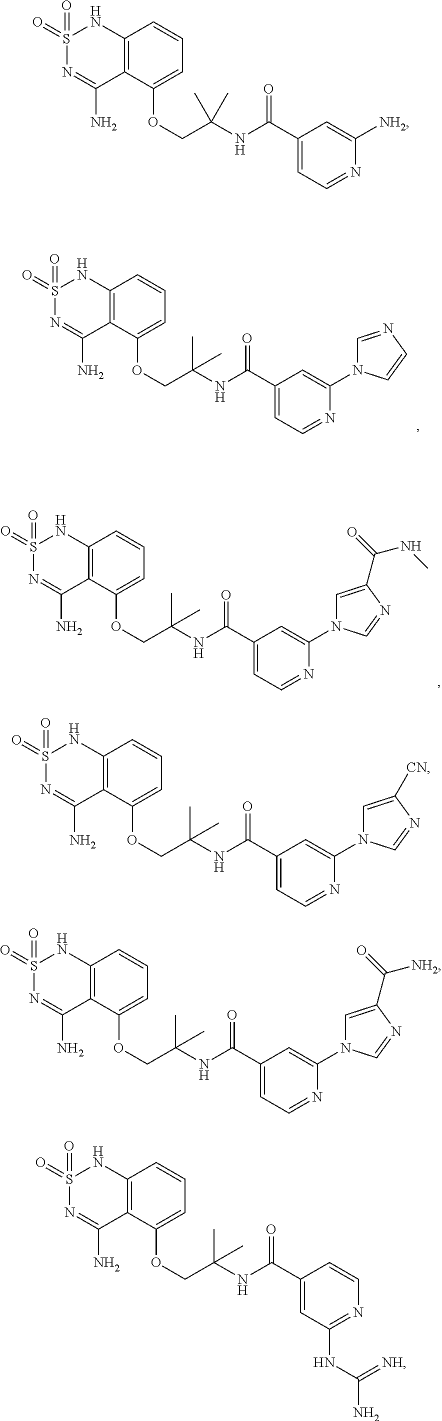 [SMILES: CC(C)(COc1cccc2c1C(N)=NS(=O)(=O)N2)NC(=O)c1ccnc(N)c1 ; CC(C)(COc1cccc2c1C(N)=NS(=O)(=O)N2)NC(=O)c1ccnc(-n2ccnc2)c1 ; CNC(=O)c1cn(-c2cc(C(=O)NC(C)(C)COc3cccc4c3C(N)=NS(=O)(=O)N4)ccn2)cn1 ; CC(C)(COc1cccc2c1C(N)=NS(=O)(=O)N2)NC(=O)c1ccnc(-n2cnc(C#N)c2)c1 ; CC(C)(COc1cccc2c1C(N)=NS(=O)(=O)N2)NC(=O)c1ccnc(-n2cnc(C(N)=O)c2)c1 ; CC(C)(COc1cccc2c1C(N)=NS(=O)(=O)N2)NC(=O)c1ccnc(NC(=N)N)c1]

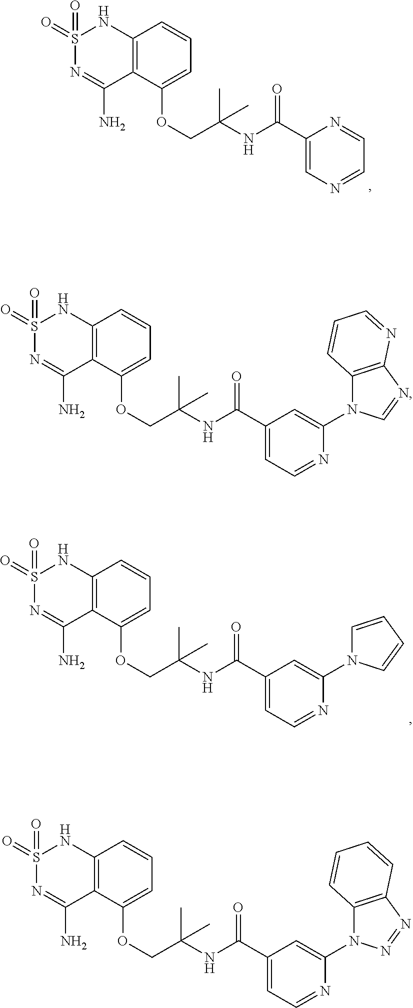 [SMILES: CC(C)(COc1cccc2c1C(N)=NS(=O)(=O)N2)NC(=O)c1cnccn1 ; CC(C)(COc1cccc2c1C(N)=NS(=O)(=O)N2)NC(=O)c1ccnc(-n2cnc3ncccc32)c1 ; CC(C)(COc1cccc2c1C(N)=NS(=O)(=O)N2)NC(=O)c1ccnc(-n2cccc2)c1 ; CC(C)(COc1cccc2c1C(N)=NS(=O)(=O)N2)NC(=O)c1ccnc(-n2nnc3ccccc32)c1]

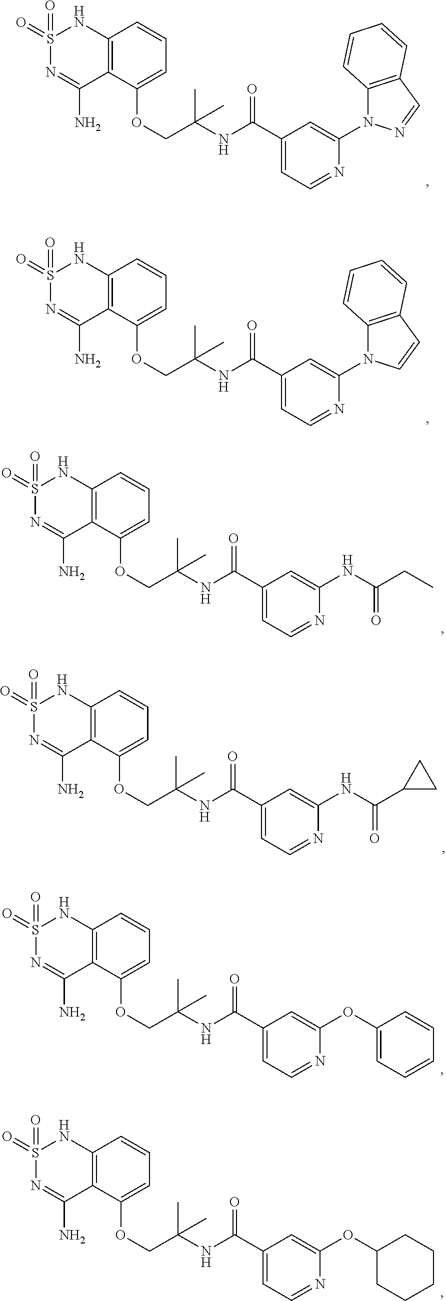 [SMILES: CC(C)(COc1cccc2c1C(N)=NS(=O)(=O)N2)NC(=O)c1ccnc(-n2ncc3ccccc32)c1 ; CC(C)(COc1cccc2c1C(N)=NS(=O)(=O)N2)NC(=O)c1ccnc(-n2ccc3ccccc32)c1 ; CCC(=O)Nc1cc(C(=O)NC(C)(C)COc2cccc3c2C(N)=NS(=O)(=O)N3)ccn1 ; CC(C)(COc1cccc2c1C(N)=NS(=O)(=O)N2)NC(=O)c1ccnc(NC(=O)C2CC2)c1 ; CC(C)(COc1cccc2c1C(N)=NS(=O)(=O)N2)NC(=O)c1ccnc(Oc2ccccc2)c1 ; CC(C)(COc1cccc2c1C(N)=NS(=O)(=O)N2)NC(=O)c1ccnc(OC2CCCCC2)c1]

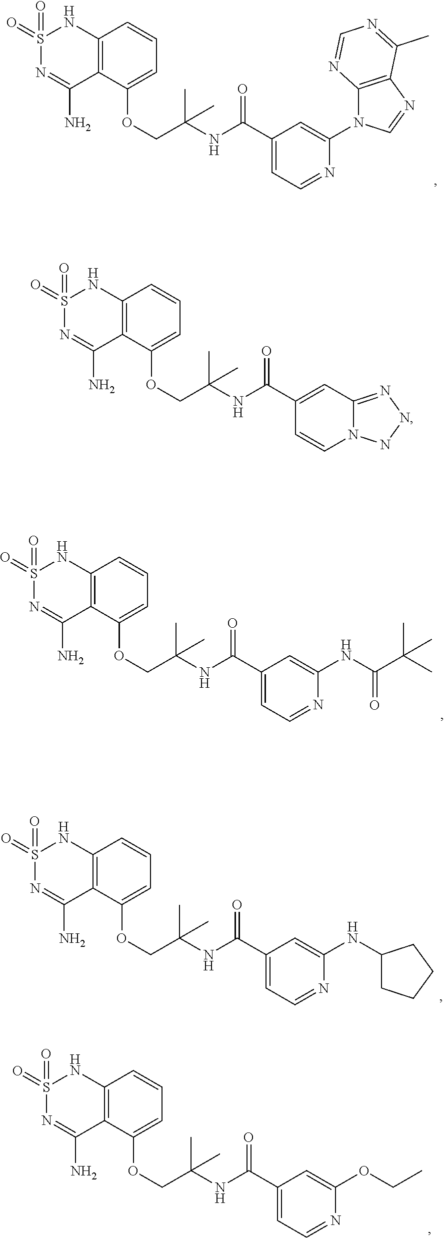

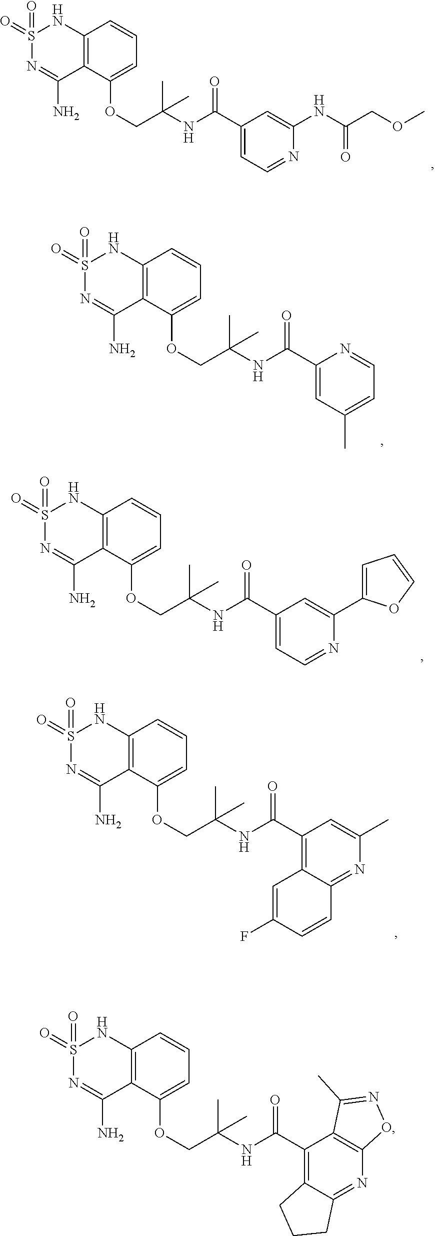 [SMILES: COCC(=O)Nc1cc(C(=O)NC(C)(C)COc2cccc3c2C(N)=NS(=O)(=O)N3)ccn1 ; Cc1ccnc(C(=O)NC(C)(C)COc2cccc3c2C(N)=NS(=O)(=O)N3)c1 ; CC(C)(COc1cccc2c1C(N)=NS(=O)(=O)N2)NC(=O)c1ccnc(-c2ccco2)c1 ; Cc1cc(C(=O)NC(C)(C)COc2cccc3c2C(N)=NS(=O)(=O)N3)c2cc(F)ccc2n1 ; Cc1noc2nc3c(c(C(=O)NC(C)(C)COc4cccc5c4C(N)=NS(=O)(=O)N5)c12)CCC3]

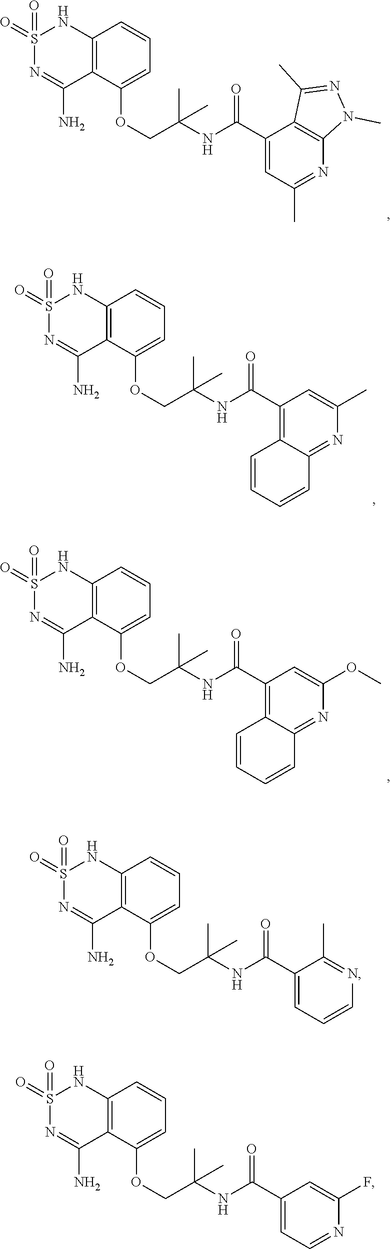 [SMILES: Cc1cc(C(=O)NC(C)(C)COc2cccc3c2C(N)=NS(=O)(=O)N3)c2c(C)nn(C)c2n1 ; Cc1cc(C(=O)NC(C)(C)COc2cccc3c2C(N)=NS(=O)(=O)N3)c2ccccc2n1 ; COc1cc(C(=O)NC(C)(C)COc2cccc3c2C(N)=NS(=O)(=O)N3)c2ccccc2n1 ; Cc1ncccc1C(=O)NC(C)(C)COc1cccc2c1C(N)=NS(=O)(=O)N2 ; CC(C)(COc1cccc2c1C(N)=NS(=O)(=O)N2)NC(=O)c1ccnc(F)c1]

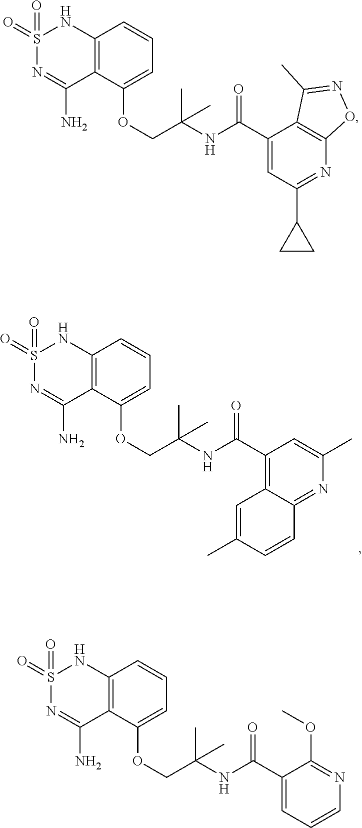 [SMILES: Cc1noc2nc(C3CC3)cc(C(=O)NC(C)(C)COc3cccc4c3C(N)=NS(=O)(=O)N4)c12 ; Cc1ccc2nc(C)cc(C(=O)NC(C)(C)COc3cccc4c3C(N)=NS(=O)(=O)N4)c2c1 ; COc1ncccc1C(=O)NC(C)(C)COc1cccc2c1C(N)=NS(=O)(=O)N2]

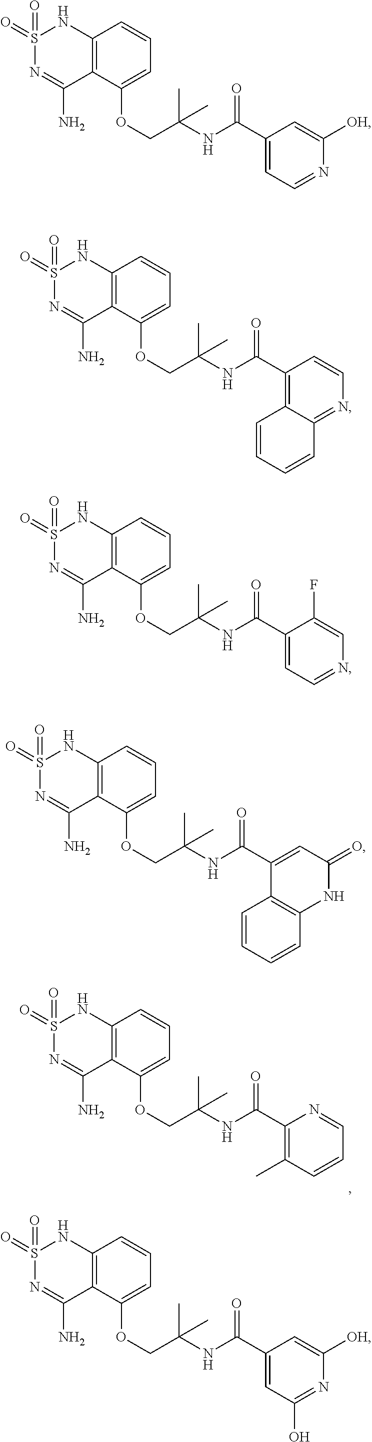 [SMILES: CC(C)(COc1cccc2c1C(N)=NS(=O)(=O)N2)NC(=O)c1ccnc(O)c1 ; CC(C)(COc1cccc2c1C(N)=NS(=O)(=O)N2)NC(=O)c1ccnc2ccccc12 ; CC(C)(COc1cccc2c1C(N)=NS(=O)(=O)N2)NC(=O)c1ccncc1F ; CC(C)(COc1cccc2c1C(N)=NS(=O)(=O)N2)NC(=O)c1cc(=O)[nH]c2ccccc12 ; Cc1cccnc1C(=O)NC(C)(C)COc1cccc2c1C(N)=NS(=O)(=O)N2 ; CC(C)(COc1cccc2c1C(N)=NS(=O)(=O)N2)NC(=O)c1cc(O)nc(O)c1]

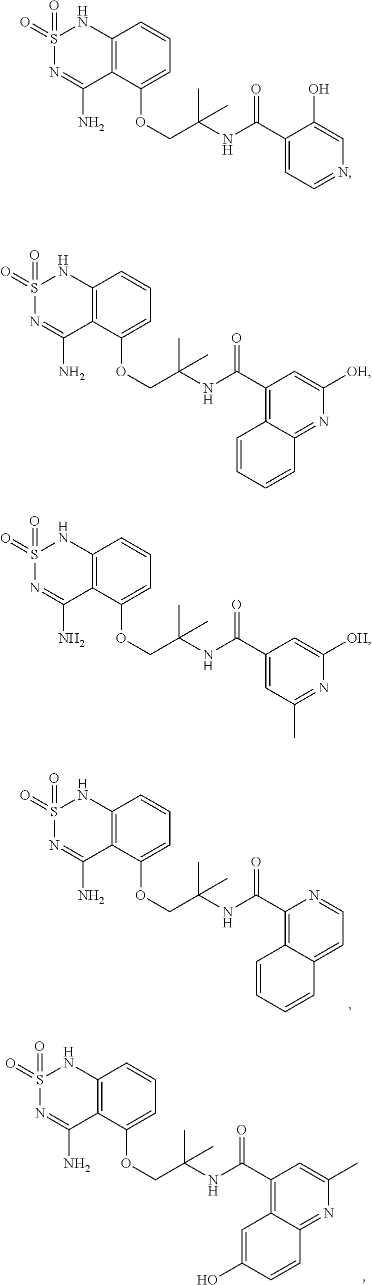 [SMILES: CC(C)(COc1cccc2c1C(N)=NS(=O)(=O)N2)NC(=O)c1ccncc1O ; CC(C)(COc1cccc2c1C(N)=NS(=O)(=O)N2)NC(=O)c1cc(O)nc2ccccc12 ; Cc1cc(C(=O)NC(C)(C)COc2cccc3c2C(N)=NS(=O)(=O)N3)cc(O)n1 ; CC(C)(COc1cccc2c1C(N)=NS(=O)(=O)N2)NC(=O)c1nccc2ccccc12 ; Cc1cc(C(=O)NC(C)(C)COc2cccc3c2C(N)=NS(=O)(=O)N3)c2cc(O)ccc2n1]

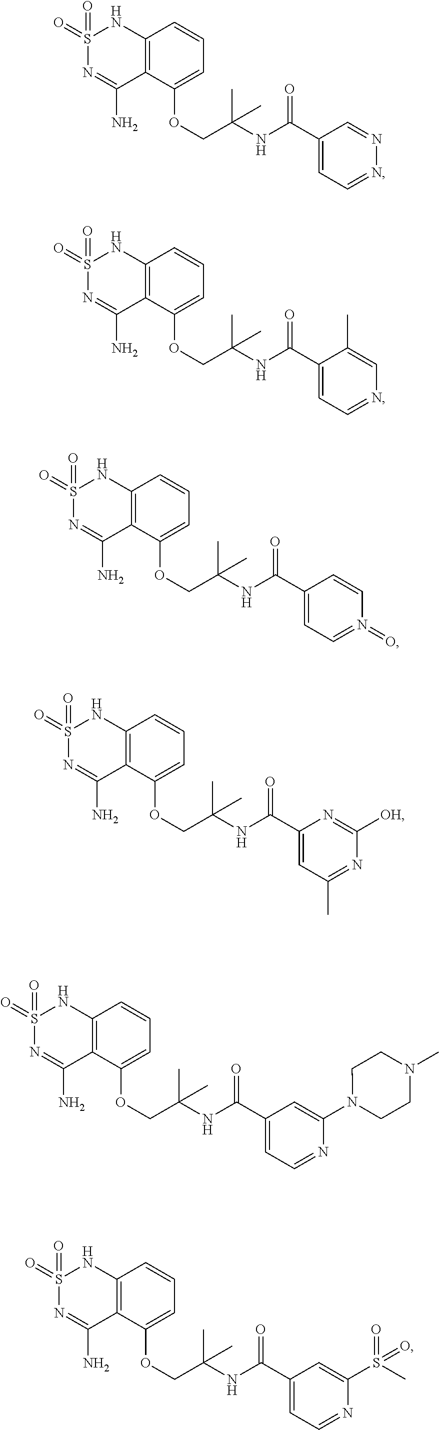 [SMILES: CC(C)(COc1cccc2c1C(N)=NS(=O)(=O)N2)NC(=O)c1ccnnc1 ; Cc1cnccc1C(=O)NC(C)(C)COc1cccc2c1C(N)=NS(=O)(=O)N2 ; CC(C)(COc1cccc2c1C(N)=NS(=O)(=O)N2)NC(=O)c1cc[n+]([O-])cc1 ; Cc1cc(C(=O)NC(C)(C)COc2cccc3c2C(N)=NS(=O)(=O)N3)nc(O)n1 ; CN1CCN(c2cc(C(=O)NC(C)(C)COc3cccc4c3C(N)=NS(=O)(=O)N4)ccn2)CC1 ; CC(C)(COc1cccc2c1C(N)=NS(=O)(=O)N2)NC(=O)c1ccnc(S(C)(=O)=O)c1]

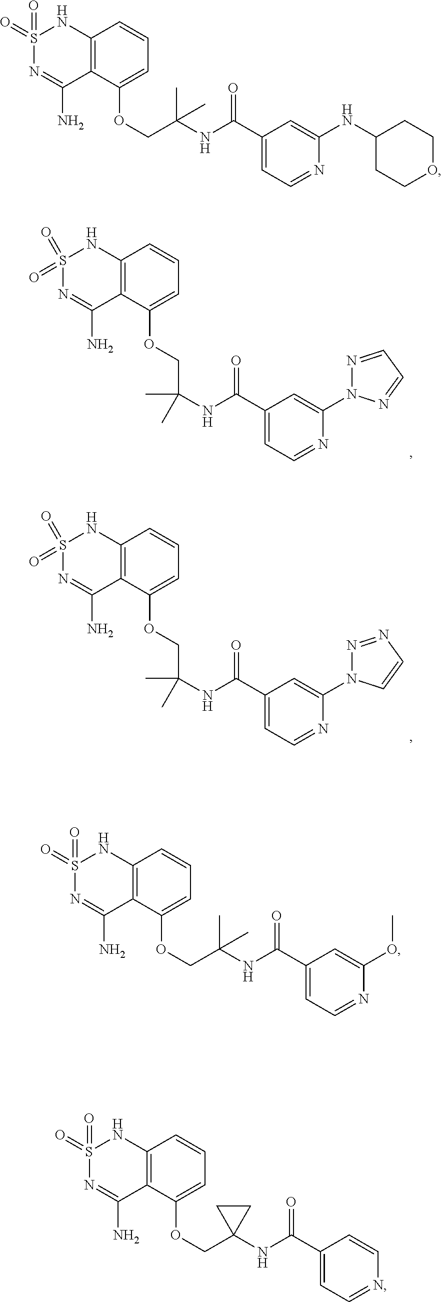 [SMILES: CC(C)(COc1cccc2c1C(N)=NS(=O)(=O)N2)NC(=O)c1ccnc(NC2CCOCC2)c1 ; CC(C)(COc1cccc2c1C(N)=NS(=O)(=O)N2)NC(=O)c1ccnc(-n2nccn2)c1 ; CC(C)(COc1cccc2c1C(N)=NS(=O)(=O)N2)NC(=O)c1ccnc(-n2ccnn2)c1 ; COc1cc(C(=O)NC(C)(C)COc2cccc3c2C(N)=NS(=O)(=O)N3)ccn1 ; NC1=NS(=O)(=O)Nc2cccc(OCC3(NC(=O)c4ccncc4)CC3)c21]

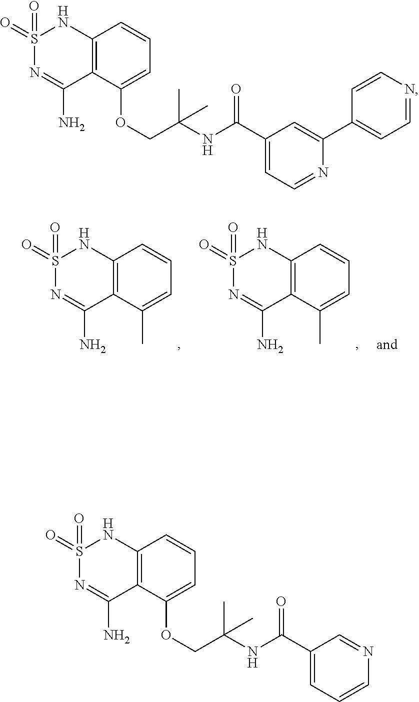

or a salt or solvate thereof.

In some embodiments, the flavor modifying compound is present at an amount from about 0.001 ppm to 500 ppm. Thus, in some alternatives, the amount of the flavor modifying compound is 0.001, 0.01, 0.1, 1, 10, 50, 100, 200, 300, 400, or 500 ppm or a value that is within a range defined by any two of the aforementioned values.

### Compositions and Uses

Some compositions herein relate to formulations comprising one or more sweetener as disclosed herein in combination with one or more flavor modifying compounds as described herein. Thus, it is recognized that the combinations of one or more sweeteners with one or more flavor modifying compounds as disclosed herein are non-limiting. In some embodiments, said formulations comprising one or more sweetener and one or more flavor modifying compounds are used as compositions for ingestible or non-ingestible products.

A formulation comprising one or more sweetener in combination with one or more flavor modifying compound can be used for modulating a chemosensory receptor and/or its ligand, including modulating the activity, structure, function, expression, and/or modification of a chemosensory receptor as well as modulating, treating, or taking prophylactic measure of a condition, e.g., physiological or pathological condition, associated with a chemosensory receptor. In general, a physiological or pathological condition associated with a chemosensory receptor includes a condition, disease, or disorder associated with the chemosensory receptor and/or its ligand, e.g., gastrointestinal disorders, metabolic disorders, functional gastrointestinal disorders, etc. In some embodiments, the formulation increases or enhances sweet flavor. In another embodiment, the formulation modulates a sweet receptor and/or its ligand expressed in a place of the body other than the taste buds, such as an internal organ. In general, the formulations of the present disclosure can be provided in a composition, such as, e.g., an ingestible composition. In some embodiments, the present formulation can impart a more sugar-like temporal profile and/or flavor profile to a sweetener composition by combining one or more flavor modifying compound with one or more sweetener in the combined formulation. In another embodiment, the present formulation can increase or enhance the sweet taste of a composition by contacting the composition thereof with one or more present formulation to form a modified composition. In another embodiment, the present formulations can be in a composition that modulates the sweet receptors and/or their ligands expressed in the body other than in the taste buds.

In some embodiments, the present formulations have sucrose modifying behavior and/or sweet agonist behavior in vitro and/or in vivo (e.g., as shown in sensory studies). In some embodiments, the present formulations demonstrate a favorable side-taste profile in vivo.

Whether or not a formulation exhibits sweet modifying/agonist effects can be determined by any suitable test method. For example, the formulation comprising one or more sweeteners in combination with a flavor modifying compound can be evaluated in a sensory test using a human taste panel.

In some embodiments, the formulation may be diluted before being tested. In some embodiments, the formulation is diluted for about 2 times, about 5, about 10, about 50, about 100, about 200, about 300, about 400, about 500, about 1000, or more times before being tested.

The tests can be conducted with and/or without additives. In some embodiments, the formulation is tested to evaluate the sweet enhancement to one or more additives. In the test, the participants can provide their impression as to the similarities of the characteristics of the sweetener compositions, with and/or without additives, with those comprising sugar. A suitable procedure for determining whether a composition has a more sugar-like taste is through the use of a panel of assessors, who measure the sweetness of the formulations.

One embodiment provides a formulation for use in a method of preparing a ready-to-use composition, such as a final food or beverage product, or animal feed product. The method comprises contacting a first composition, such as a first food or beverage product that may contain a sweetener described herein, with a flavoring concentrate composition or formulation (e.g., solid or liquid) comprising a flavor modifying compound to form the ready-to-use composition.

In some embodiments, an ingestible composition may be a beverage. In some embodiments, the beverage may be selected from the group consisting of enhanced sparkling beverages, colas, lemon-lime flavored sparkling beverages, orange flavored sparkling beverages, grape flavored sparkling beverages, strawberry flavored sparkling beverages, pineapple flavored sparkling beverages, ginger-ales, root beers, fruit juices, fruit-flavored juices, juice drinks, nectars, vegetable juices, vegetable-flavored juices, sports drinks, energy drinks, enhanced water drinks, enhanced water with vitamins, near water drinks, coconut waters, tea type drinks, coffees, cocoa drinks, beverages containing milk components, chocolate milk, dairy only flavored milk drinks, flavored milk drinks with fruit juice, milk alternative beverages including rice milk, almond milk, cashew milk, coconut milk, hazelnut milk, hemp milk, pistachio milk, oat milk, wheat milk, barley milk, millet milk, spelt milk, triticale milk, and soy milk, sour milk drinks, fermented dairy drinks, kefir, beverages containing cereal extracts and smoothies. In some embodiments, the beverage may be a soft drink.

In some embodiments, one or more flavor modifying compound as described herein and one or more sweetener as described herein may be included in a food or beverage product, wherein the food or beverage product may additionally comprise:

acids, including, for example citric acid, phosphoric acid, ascorbic acid, sodium acid sulfate, lactic acid, or tartaric acid;

bitter ingredients, including, for example caffeine, quinine, green tea, catechins, polyphenols, green robusta coffee extract, green coffee extract, whey protein isolate, or potassium chloride;

coloring agents, including, for example caramel color, Red #40, Yellow #5, Yellow #6, Blue #1, Red #3, purple carrot, black carrot juice, purple sweet potato, vegetable juice, fruit juice, beta carotene, turmeric curcumin, or titanium dioxide;

preservatives, including, for example sodium benzoate, potassium benzoate, potassium sorbate, sodium metabisulfate, sorbic acid, or benzoic acid;

antioxidants including, for example ascorbic acid, calcium disodium EDTA, alpha tocopherols, mixed tocopherols, rosemary extract, grape seed extract, resveratrol, or sodium hexametaphosphate;

vitamins or functional ingredients including, for example resveratrol, Co-Q10, omega 3 fatty acids, theanine, choline chloride (citocoline), fibersol, inulin (chicory root), taurine, panax ginseng extract, guanana extract, ginger extract, L-phenylalanine, L-carnitine, L-tartrate, D-glucoronolactone, inositol, bioflavonoids, Echinacea, ginko biloba, yerba mate, flax seed oil, garcinia cambogia rind extract, white tea extract, ribose, milk thistle extract, grape seed extract, pyrodixine HCl (vitamin B6), cyanoobalamin (vitamin B12), niacinamide (vitamin B3), biotin, calcium lactate, calcium pantothenate (pantothenic acid), calcium phosphate, calcium carbonate, chromium chloride, chromium polynicotinate, cupric sulfate, folic acid, ferric pyrophosphate, iron, magnesium lactate, magnesium carbonate, magnesium sulfate, monopotassium phosphate, monosodium phosphate, phosphorus, potassium iodide, potassium phosphate, riboflavin, sodium sulfate, sodium gluconate, sodium polyphosphate, sodium bicarbonate, thiamine mononitrate, vitamin D3, vitamin A palmitate, zinc gluconate, zinc lactate, or zinc sulphate;

clouding agents, including, for example ester gun, brominated vegetable oil (BVO), or sucrose acetate isobutyrate (SAIB);

buffers, including, for example sodium citrate, potassium citrate, or salt;

flavors, including, for example propylene glycol, ethyl alcohol, glycerine, gum Arabic (gum acacia), maltodextrin, modified corn starch, dextrose, natural flavor, natural flavor with other natural flavors (natural flavor WONF), natural and artificial flavors, artificial flavor, silicon dioxide, magnesium carbonate, or tricalcium phosphate; and

stabilizers, including, for example pectin, xanthan gum, carboxylmethylcellulose (CMC), polysorbate 60, polysorbate 80, medium chain triglycerides, cellulose gel, cellulose gum, sodium caseinate, modified food starch, gum Arabic (gum acacia), or carrageenan.

Some embodiments provide supplements, nutraceuticals, functional food products (e.g., any fresh or processed food claimed to have a health-promoting and/or disease-preventing properties beyond the basic nutritional function of supplying nutrients), animal feed products, pharmaceutical product, over the counter (OTC) product, oral care product, cosmetic products such as sweetened lip balms, and other personal care products including a flavor modifying compound as described herein and sweetener as described herein.

In general, over the counter (OTC) product and oral care product generally refer to product for household and/or personal use which may be sold without a prescription and/or without a visit to a medical professional. Examples of the OTC products include, but are not limited to Vitamins and dietary supplements; Topical analgesics and/or anesthetic; Cough, cold and allergy remedies; Antihistamines and/or allergy remedies; and combinations thereof. Vitamins and dietary supplements include, but are not limited to vitamins, dietary supplements, tonics/bottled nutritive drinks, child-specific vitamins, dietary supplements, any other products of or relating to or providing nutrition, and combinations thereof. Topical analgesics and/or anesthetic include any topical creams/ointments/gels used to alleviate superficial or deep-seated aches and pains, e.g., muscle pain; teething gel; patches with analgesic ingredient; and combinations thereof. Cough, cold and allergy remedies include, but are not limited to decongestants, cough remedies, pharyngeal preparations, medicated confectionery, antihistamines and child-specific cough, cold and allergy remedies; and combination products. Antihistamines and/or allergy remedies include, but are not limited to any systemic treatments for hay fever, nasal allergies, insect bites and stings. Examples of oral care product include, but are not limited to mouth cleaning strips, toothpaste, toothbrushes, mouthwashes/dental rinses, denture care, mouth fresheners at-home teeth whiteners, dentifrices, and dental floss.

In some embodiments, a flavor modifying compound as described herein and sweetener as described herein may be included in food or beverage products or formulations. Examples of food and beverage products or formulations include, but are not limited to sweet coatings, frostings, or glazes for ingestible products or any entity included in the Soup category, the Dried Processed Food category, the Beverage category, the Ready Meal category, the Canned or Preserved Food category, the Frozen Processed Food category, the Chilled Processed Food category, the Snack Food category, the Baked Goods category, the Confectionery category, the Dairy Product category, the Ice Cream category, the Meal Replacement category, the Pasta and Noodle category, and the Sauces, Dressings, Condiments category, the Baby Food category, and/or the Spreads category.

In general, the Soup category refers to canned/preserved, dehydrated, instant, chilled, UHT and frozen soup. For the purpose of this definition soup(s) means a food prepared from meat, poultry, fish, vegetables, grains, fruit and other ingredients, cooked in a liquid which may include visible pieces of some or all of these ingredients. It may be clear (as a broth) or thick (as a chowder), smooth, pureed or chunky, ready-to-serve, semi-condensed or condensed and may be served hot or cold, as a first course or as the main course of a meal or as a between meal snack (sipped like a beverage). Soup may be used as an ingredient for preparing other meal components and may range from broths (consommé) to sauces (cream or cheese-based soups).

The Dehydrated and Culinary Food Category usually means: (i) Cooking aid products such as: powders, granules, pastes, concentrated liquid products, including concentrated bouillon, bouillon and bouillon like products in pressed cubes, tablets or powder or granulated form, which are sold separately as a finished product or as an ingredient within a product, sauces and recipe mixes (regardless of technology); (ii) Meal solutions products such as: dehydrated and freeze dried soups, including dehydrated soup mixes, dehydrated instant soups, dehydrated ready-to-cook soups, dehydrated or ambient preparations of ready-made dishes, meals and single serve entrees including pasta, potato and rice dishes; and (iii) Meal embellishment products such as: condiments, marinades, salad dressings, salad toppings, dips, breading, batter mixes, shelf stable spreads, barbecue sauces, liquid recipe mixes, concentrates, sauces or sauce mixes, including recipe mixes for salad, sold as a finished product or as an ingredient within a product, whether dehydrated, liquid or frozen.

The Beverage category usually means beverages, beverage mixes and concentrates, including but not limited to, carbonated and non-carbonated beverages, alcoholic and non-alcoholic beverages, ready to drink beverages, liquid concentrate formulations for preparing beverages such as sodas, and dry powdered beverage precursor mixes. The Beverage category also includes the alcoholic drinks, the soft drinks, sports drinks, isotonic beverages, and hot drinks. The alcoholic drinks include, but are not limited to beer, cider/perry, flavored alcoholic beverages, wine, and spirits. The soft drinks include, but are not limited to carbonates, such as colas and non-cola carbonates; fruit juice, such as juice, nectars, juice drinks and fruit flavored drinks; bottled water, which includes sparkling water, spring water, purified/table water, and vitamin water; functional drinks, which can be carbonated or still and include sport, energy or elixir drinks; concentrates, such as liquid and powder concentrates in ready to drink measure. The drinks, either hot or cold, include, but are not limited to coffee or ice coffee, such as fresh, instant, and combined coffee; tea or ice tea, such as black, green, white, oolong, and flavored tea; and other drinks including flavor-, malt- or plant-based powders, granules, blocks or tablets mixed with milk or water.

The Snack Food category generally refers to any food that can be a light informal meal including, but not limited to Sweet and savory snacks and snack bars. Examples of snack food include, but are not limited to fruit snacks, chips/crisps, extruded snacks, tortilla/corn chips, popcorn, pretzels, nuts and other sweet and savory snacks. Examples of snack bars include, but are not limited to granola/muesli bars, breakfast bars, energy bars, fruit bars and other snack bars.

The Baked Goods category generally refers to any edible product the process of preparing which involves exposure to heat or excessive sunlight. Examples of baked goods include, but are not limited to bread, buns, cookies, muffins, cereal, toaster pastries, pastries, waffles, tortillas, biscuits, pies, bagels, tarts, quiches, cake, any baked foods, and any combination thereof.

The Ice Cream category generally refers to frozen dessert containing cream and sugar and flavoring. Examples of ice cream include, but are not limited to: impulse ice cream; take-home ice cream; frozen yoghurt and artisanal ice cream; gelato; sorbet; sherbet; soy, oat, bean (e.g., red bean and mung bean), coconut, nut and rice-based ice creams.

The Confectionery category generally refers to edible product that is sweet to the taste. Examples of confectionery include, but are not limited to candies, gelatins, chocolate confectionery, sugar confectionery, gum, and the likes and any combination products.

The Meal Replacement category generally refers to any food intended to replace the normal meals, particularly for people having health or fitness concerns. Examples of meal replacement include, but are not limited to slimming products and convalescence products.

The Ready Meal category generally refers to any food that can be served as meal without extensive preparation or processing. The ready meal includes products that have had recipe “skills” added to them by the manufacturer, resulting in a high degree of readiness, completion and convenience. Examples of ready meal include, but are not limited to canned/preserved, frozen, dried, chilled ready meals; dinner mixes; frozen pizza; chilled pizza; and prepared salads.

The Pasta and Noodle category includes any pastas and/or noodles including, but not limited to canned, dried and chilled/fresh pasta; and plain, instant, chilled, frozen and snack noodles.

The Canned/Preserved Food category includes, but is not limited to canned/preserved meat and meat products, fish/seafood, vegetables, tomatoes, beans, fruit, ready meals, soup, pasta, and other canned/preserved foods.

The Frozen Processed Food category includes, but is not limited to frozen processed red meat, processed poultry, processed fish/seafood, processed vegetables, meat substitutes, processed potatoes, bakery products, desserts, ready meals, pizza, soup, noodles, and other frozen food.

The Dried Processed Food category includes, but is not limited to rice, dessert mixes, dried ready meals, dehydrated soup, instant soup, dried pasta, plain noodles, and instant noodles. The Chill Processed Food category includes, but is not limited to chilled processed meats, processed fish/seafood products, lunch kits, fresh cut fruits, ready meals, pizza, prepared salads, soup, fresh pasta and noodles.

The Sauces, Dressings and Condiments category includes, but is not limited to tomato pastes and purees, bouillon/stock cubes, herbs and spices, monosodium glutamate (MSG), table sauces, soy based sauces, pasta sauces, wet/cooking sauces, dry sauces/powder mixes, ketchup, mayonnaise, mustard, salad dressings, vinaigrettes, dips, pickled products, and other sauces, dressings and condiments.

The Baby Food category includes, but is not limited to milk- or soybean-based formula; and prepared, dried and other baby food.

The Spreads category includes, but is not limited to jams and preserves, honey, chocolate spreads, nut based spreads, speculoos spreads, butters, flavored butters, margarine, edible oil spreads, oleos, cheese or cream cheese spreads, savory spread, and yeast based spreads.

The Dairy Product category generally refers to edible product produced from mammal's milk. Examples of dairy product include, but are not limited to drinking milk products, cheese, yoghurt and sour milk drinks, and other dairy products.

Additional examples for ingestible compositions, particularly food and beverage products or formulations, are provided as follows. Exemplary ingestible compositions include one or more confectioneries, chocolate confectionery, tablets, countlines, bagged selflines/softlines, boxed assortments, standard boxed assortments, twist wrapped miniatures, seasonal chocolate, chocolate with toys, alfajores, other chocolate confectionery, mints, standard mints, power mints, boiled sweets, pastilles, gums, jellies and chews, toffees, caramels and nougat, medicated confectionery, lollipops, liquorice, other sugar confectionery, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savory biscuits and crackers, bread substitutes, breakfast cereals, rte cereals, family breakfast cereals, flakes, muesli, other cereals, children's breakfast cereals, hot cereals, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yoghurt, artisanal ice cream, dairy products, milk, fresh/pasteurized milk, full fat fresh/pasteurized milk, semi skimmed fresh/pasteurized milk, long-life/uht milk, full fat long life/uht milk, semi skimmed long life/uht milk, fat-free long life/uht milk, goat milk, condensed/evaporated milk, plain condensed/evaporated milk, flavored, functional and other condensed milk, flavored milk drinks, chocolate milk, dairy only flavored milk drinks, flavored milk drinks with fruit juice, milk alternative beverages including rice milk, almond milk, cashew milk, coconut milk, hazelnut milk, hemp milk, pistachio milk, oat milk, wheat milk, barley milk, millet milk, spelt milk, triticale milk, and soy milk, sour milk drinks, fermented dairy drinks, kefir, coffee whiteners, powder milk, flavored powder milk drinks, cream, cheese, processed cheese, spreadable processed cheese, unspreadable processed cheese, unprocessed cheese, spreadable unprocessed cheese, hard cheese, packaged hard cheese, unpackaged hard cheese, yoghurt, plain/natural yoghurt, flavored yoghurt, fruited yoghurt, probiotic yoghurt, drinking yoghurt, regular drinking yoghurt, probiotic drinking yoghurt, chilled and shelf-stable desserts, dairy-based desserts, soy-based desserts, chilled snacks, fromage frais and quark, plain fromage frais and quark, flavored fromage frais and quark, savory fromage frais and quark, sweet and savory snacks, fruit snacks, chips/crisps, extruded snacks, tortilla/corn chips, popcorn, pretzels, nuts, other sweet and savory snacks, snack bars, granola bars, breakfast bars, energy bars, fruit bars, other snack bars, meal replacement products, slimming products, convalescence drinks, ready meals, canned ready meals, frozen ready meals, dried ready meals, chilled ready meals, dinner mixes, frozen pizza, chilled pizza, soup, canned soup, dehydrated soup, instant soup, chilled soup, hot soup, frozen soup, pasta, canned pasta, dried pasta, chilled/fresh pasta, noodles, plain noodles, instant noodles, cups/bowl instant noodles, pouch instant noodles, chilled noodles, snack noodles, canned food, canned meat and meat products, canned fish/seafood, canned vegetables, canned tomatoes, canned beans, canned fruit, canned ready meals, canned soup, canned pasta, other canned foods, frozen food, frozen processed red meat, frozen processed poultry, frozen processed fish/seafood, frozen processed vegetables, frozen meat substitutes, frozen potatoes, oven baked potato chips, other oven baked potato products, non-oven frozen potatoes, frozen bakery products, frozen desserts, frozen ready meals, frozen pizza, frozen soup, frozen noodles, other frozen food, dried food, dessert mixes, dried ready meals, dehydrated soup, instant soup, dried pasta, plain noodles, instant noodles, cups/bowl instant noodles, pouch instant noodles, chilled food, chilled processed meats, chilled fish/seafood products, chilled processed fish, chilled coated fish, chilled smoked fish, chilled lunch kit, chilled ready meals, chilled pizza, chilled soup, chilled/fresh pasta, chilled noodles, oils and fats, olive oil, vegetable and seed oil, cooking fats, butter, margarine, spreadable oils and fats, functional spreadable oils and fats, sauces, dressings and condiments, tomato pastes and purees, bouillon/stock cubes, stock cubes, gravy granules, liquid stocks and fonds, herbs and spices, fermented sauces, soy based sauces, pasta sauces, wet sauces, dry sauces/powder mixes, ketchup, mayonnaise, regular mayonnaise, mustard, salad dressings, regular salad dressings, low fat salad dressings, vinaigrettes, dips, pickled products, other sauces, dressings and condiments, baby food, milk formula, standard milk formula, follow-on milk formula, toddler milk formula, hypoallergenic milk formula, prepared baby food, dried baby food, other baby food, spreads, jams and preserves, honey, chocolate spreads, nut-based spreads, speculoos spreads, butters, flavored butters, margarine, edible oil spreads, oleos, cheese or cream cheese spreads, savory spread, and yeast-based spreads. Exemplary ingestible compositions also include confectioneries, bakery products, ice creams, dairy products, sweet and savory snacks, snack bars, meal replacement products, ready meals, soups, pastas, noodles, canned foods, frozen foods, dried foods, chilled foods, oils and fats, baby foods, or spreads or a mixture thereof. Exemplary ingestible compositions also include breakfast cereals, sweet beverages or solid or liquid concentrate compositions for preparing beverages, ideally so as to enable the reduction in concentration of previously known saccharide sweeteners, or artificial sweeteners.

Some embodiments provide a chewable composition that may or may not be intended to be swallowed. In some embodiments, the chewable composition may be gum, chewing gum, sugarized gum, sugar-free gum, functional gum, bubble gum including a flavor modifying compound as described herein and sweetener as described herein.

In some embodiments, a flavor modifying compound as described herein and sweetener as described herein may be provided in a flavoring concentrate formulation, e.g., suitable for subsequent processing to produce a ready-to-use (for example, ready-to-serve) product. By “a flavoring concentrate formulation”, it is meant a formulation which should be reconstituted with one or more diluting medium to become a ready-to-use composition. The term “ready-to-use composition” is used herein interchangeably with “ingestible composition”, which denotes any substance that, either alone or together with another substance, can be taken by mouth whether intended for consumption or not. In one embodiment, the ready-to-use composition includes a composition that can be directly consumed by a human or animal. The flavoring concentrate formulation is typically used by mixing with or diluted by one or more diluting medium, e.g., any consumable or ingestible ingredient or product, to impart or modify one or more flavors to the diluting medium. Such a use process is often referred to as reconstitution. The reconstitution can be conducted in a household setting or an industrial setting. For example, a frozen fruit juice concentrate can be reconstituted with water or other aqueous medium by a consumer in a kitchen to obtain the ready-to-use fruit juice beverage. In another example, a soft drink syrup concentrate can be reconstituted with water or other aqueous medium by a manufacturer in large industrial scales to produce the ready-to-use soft drinks. Since the flavoring concentrate formulation has the flavoring agent or flavor modifying agent in a concentration higher than the ready-to-use composition, the flavoring concentrate formulation is typically not suitable for being consumed directly without reconstitution. There are many benefits of using and producing a flavoring concentrate formulation. For example, one benefit is the reduction in weight and volume for transportation as the flavoring concentrate formulation can be reconstituted at the time of usage by the addition of suitable solvent, solid or liquid.

In one embodiment, the flavoring concentrate formulation comprises i) one or more flavor modifying compound as described herein; ii) a carrier; and iii) optionally at least one adjuvant. The term “carrier” denotes a usually inactive accessory substance, such as solvents, binders, or other inert medium, which is used in combination with the flavor modifying compound and one or more optional adjuvants to form the formulation. For example, water or starch can be a carrier for a flavoring concentrate formulation. In some embodiments, the carrier is the same as the diluting medium for reconstituting the flavoring concentrate formulation; and in other embodiments, the carrier is different from the diluting medium. The term “carrier” as used herein includes, but is not limited to, ingestibly acceptable carrier.

The term “adjuvant” denotes an additive which supplements, stabilizes, maintains, or enhances the intended function or effectiveness of the active ingredient, such as the compound of the present disclosure. In one embodiment, the at least one adjuvant comprises one or more flavoring agents. The flavoring agent may be of any flavor known to one skilled in the art or consumers, such as the flavor of chocolate, coffee, tea, mocha, French vanilla, peanut butter, chai, or combinations thereof. In another embodiment, the at least one adjuvant comprises one or more sweeteners. The one or more sweeteners can be any of the sweeteners described above. In another embodiment, the at least one adjuvant comprises one or more ingredients selected from the group consisting of a emulsifier, a stabilizer, an antimicrobial preservative, an antioxidant, vitamins, minerals, fats, starches, protein concentrates and isolates, salts, and combinations thereof. Examples of emulsifiers, stabilizers, antimicrobial preservatives, antioxidants, vitamins, minerals, fats, starches, protein concentrates and isolates, and salts are described in U.S. Pat. No. 6,468,576, the content of which is hereby incorporated by reference in its entirety for all purposes.

In one embodiment, the present flavoring concentrate formulation can be in a form selected from the group consisting of liquid including solution and suspension, solid, foamy material, paste, gel, cream, and a combination thereof, such as a liquid containing certain amount of solid contents. In one embodiment, the flavoring concentrate formulation is in form of a liquid including aqueous-based and nonaqueous-based. In some embodiments, the present flavoring concentrate formulation can be carbonated or non-carbonated.

The flavoring concentrate formulation may further comprise a freezing point depressant, nucleating agent, or both as the at least one adjuvant. The freezing point depressant is an ingestibly acceptable compound or agent which can depress the freezing point of a liquid or solvent to which the compound or agent is added. That is, a liquid or solution containing the freezing point depressant has a lower freezing point than the liquid or solvent without the freezing point depressant. In addition to depress the onset freezing point, the freezing point depressant may also lower the water activity of the flavoring concentrate formulation. The examples of the freezing point depressant include, but are not limited to, carbohydrates, oils, ethyl alcohol, polyol, e.g., glycerol, and combinations thereof. The nucleating agent denotes an ingestibly acceptable compound or agent which is able to facilitate nucleation. The presence of nucleating agent in the flavoring concentrate formulation can improve the mouthfeel of the frozen Blushes of a frozen slush and to help maintain the physical properties and performance of the slush at freezing temperatures by increasing the number of desirable ice crystallization centers. Examples of nucleating agents include, but are not limited to, calcium silicate, calcium carbonate, titanium dioxide, and combinations thereof.

In one embodiment, the flavoring concentrate formulation is formulated to have a low water activity for extended shelf life. Water activity is the ratio of the vapor pressure of water in a formulation to the vapor pressure of pure water at the same temperature. In one embodiment, the flavoring concentrate formulation has a water activity of less than about 0.85. In another embodiment, the flavoring concentrate formulation has a water activity of less than about 0.80. In another embodiment, the flavoring concentrate formulation has a water activity of less than about 0.75.

In one embodiment, the flavoring concentrate formulation has the flavor modifying compound in a concentration that is at least 2 times of the concentration of the compound in a ready-to-use composition. In one embodiment, the flavoring concentrate formulation has the flavor modifying compound in a concentration that is at least 5 times of the concentration of the compound in a ready-to-use composition. In one embodiment, the flavoring concentrate formulation has the flavor modifying compound in a concentration that is at least 10 times of the concentration of the compound in a ready-to-use composition. In one embodiment, the flavoring concentrate formulation has the flavor modifying compound in a concentration that is at least 15 times of the concentration of the compound in a ready-to-use composition. In one embodiment, the flavoring concentrate formulation has the flavor modifying compound in a concentration that is at least 20 times of the concentration of the compound in a ready-to-use composition. In one embodiment, the flavoring concentrate formulation has the flavor modifying compound in a concentration that is at least 30 times of the concentration of the compound in a ready-to-use composition. In one embodiment, the flavoring concentrate formulation has the flavor modifying compound in a concentration that is at least 40 times of the concentration of the compound in a ready-to-use composition. In one embodiment, the flavoring concentrate formulation has the flavor modifying compound in a concentration that is at least 50 times of the concentration of the compound in a ready-to-use composition. In one embodiment, the flavoring concentrate formulation has the flavor modifying compound in a concentration that is at least 60 times of the concentration of the compound in a ready-to-use composition. In one embodiment, the flavoring concentrate formulation has the flavor modifying compound in a concentration that is up to 100 times of the concentration of the compound in a ready-to-use composition.

The foregoing detailed description has been given for clearness of understanding only and no unnecessary limitations should be understood therefrom as modifications will be obvious to those skilled in the art. It is not an admission that any of the information provided herein is prior art or relevant to the presently claimed embodiments, or that any publication specifically or implicitly referenced is prior art.

What is claimed is:

1. A formulation comprising one or more sweetener and one or more flavor modifying compound.

2. The formulation of claim 1, wherein the flavor modifying compound is compound having a cyclic thiadiazine core.

3. The formulation of claim 1, wherein the flavor modifying compound is: 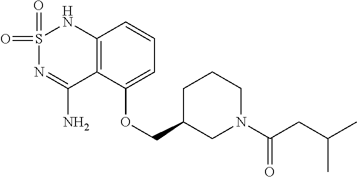 [SMILES: CC(C)CC(=O)N1CCC[C@H](COc2cccc3c2C(N)=NS(=O)(=O)N3)C1]
or a salt or solvate thereof.

4. The formulation of claim 1, wherein the flavor modifying compound is: 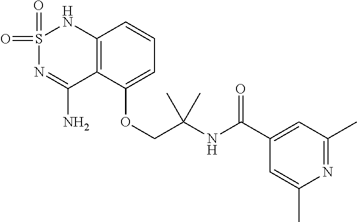 [SMILES: Cc1cc(C(=O)NC(C)(C)COc2cccc3c2C(N)=NS(=O)(=O)N3)cc(C)n1]
or a salt or solvate thereof.

5. The formulation of claim 1, which is an ingestible composition.

6. The ingestible composition of claim 5, which is in the form of a food or beverage product, an animal feed product, a pharmaceutical composition, a nutritional product, a dietary supplement, or over-the-counter medication.

7. The formulation of claim 1, wherein the one or more sweetener is a natural or artificial compound.

8. The formulation of claim 1, wherein the one or more sweetener is selected from the group consisting of:
agave inulin, agave nectar, agave syrup, amazake, brazzein, brown rice syrup, coconut crystals, coconut sugars, coconut syrup, date sugar, fructans (also referred to as inulin fiber, fructo-oligosaccharides, or oligo-fructose), green stevia powder, stevia rebaudiana, rebaudioside A, rebaudioside B, rebaudioside C, rebaudioside D, rebaudioside E, rebaudioside F, rebaudioside I, rebaudioside H, rebaudioside L, rebaudioside K, rebaudioside J, rebaudioside N, rebaudioside O, rebaudioside M and other sweet stevia-based glycosides, stevioside, stevioside extracts, honey, Jerusalem artichoke syrup, licorice root, luo han guo (fruit, powder, or extracts), lucuma (fruit, powder, or extracts), maple sap (including, for example, sap extracted from Acer saccharum, Acer nigrum, Acer rubrum, Acer saccharinum, Acer platanoides, Acer negundo, Acer macrophyllum, Acer grandidentatum, Acer glabrum, Acer mono), maple syrup, maple sugar, walnut sap (including, for example, sap extracted from Juglans cinerea, Juglans nigra, Juglans ailatifolia, Juglans regia), birch sap (including, for example, sap extracted from Betula papyrifera, Betula alleghaniensis, Betula lenta, Betula nigra, Betula populifolia, Betula pendula), sycamore sap (such as, for example, sap extracted from Platanus occidentalis), ironwood sap (such as, for example, sap extracted from Ostrya virginiana), mascobado, molasses (such as, for example, blackstrap molasses), molasses sugar, monatin, monellin, cane sugar (also referred to as natural sugar, unrefined cane sugar, or sucrose), palm sugar, panocha, piloncillo, rapadura, raw sugar, rice syrup, sorghum, sorghum syrup, cassava syrup (also referred to as tapioca syrup), thaumatin, yacon root, malt syrup, barley malt syrup, barley malt powder, beet sugar, cane sugar, crystalline juice crystals, caramel, carbitol, carob syrup, castor sugar, hydrogenated starch hydrolates, hydrolyzed can juice, hydrolyzed starch, invert sugar, anethole, arabinogalactan, arrope, syrup, P-4000, acesulfame potassium (also referred to as acesulfame K or ace-K), alitame (also referred to as aclame), advantame, aspartame, baiyunoside, neotame, benzamide derivatives, bernadame, canderel, carrelame and other guanidine-based sweeteners, vegetable fiber, corn sugar, coupling sugars, curculin, cyclamates, cyclocarioside I, demerara, dextran, dextrin, diastatic malt, dulcin, sucrol, valzin, dulcoside A, dulcoside B, emulin, enoxolone, maltodextrin, saccharin, estragole, ethyl maltol, glucin, gluconic acid, glucono-lactone, glucosamine, glucoronic acid, glycerol, glycine, glycyphillin, glycyrrhizin, golden sugar, yellow sugar, golden syrup, granulated sugar, gynostemma, hernandulcin, isomerized liquid sugars, jallab, chicory root dietary fiber, kynurenine derivatives (including N′-formyl-kynurenine, N′-acetyl-kynurenine, 6-chloro-kynurenine), galactitol, litesse, ligicane, lycasin, lugduname, guanidine, falernum, mabinlin I, mabinlin II, maltol, maltisorb, maltodextrin, maltotriol, mannosamine, miraculin, mizuame, mogrosides (including, for example, mogroside I (including mogrosides I_{A} and I_{E}), mogroside II (including mogrosides II_{A}, II_{A1}, II_{A2}, II_{B}, and II_{E}), mogroside Ill (including mogroside III_{A2}, III_{E}), mogroside IV (including mogrosides IV_{A} and IV_{E}), mogroside V, and mogroside VI), 7-oxomogrosides (including, for example, 7-oxomogroside II_{E} and 7-oxomogroside V), 11-deoxymogrosides (including, for example, 11-deoxymogrosides III and V), 11-oxomogrosides (including, for example, 11-oxomogrosides I_{A}, III_{E}, IV, IV_{A}, IV_{E}, V, and VI), siamenoside, siamenoside I, isomogroside, isomogroside V, mogrol, 11-oxomogrol, neomogroside, mukurozioside, nano sugar, naringin dihydrochalcone, neohesperidine dihydrochalcone, nib sugar, nigero-oligosaccharide, norbu, orgeat syrup, osladin, pekmez, pentadin, periandrin I, perillaldehyde, perillartine, petphyllum, phenylalanine, phlomisoside I, phlorodizin, phyllodulcin, polyglycitol syrups, polypodoside A, pterocaryoside A, pterocaryoside B, rebiana, refiners syrup, rub syrup, rubusoside, selligueain A, shugr, siamenoside I, siraitia grosvenorii, soybean oligosaccharide, Splenda, SRI oxime V, steviol glycoside, steviolbioside, stevioside, strogins 1, 2, and 4, sucronic acid, sucrononate, sugar, suosan, phloridzin, superaspartame, tetrasaccharide, threitol, treacle, trilobtain, tryptophan and derivatives (6-trifluoromethyl-tryptophan, 6-chloro-D-tryptophan), vanilla sugar, volemitol, birch syrup, aspartame-acesulfame, assugrin, and combinations or blends of any two or more thereof.

9. The ingestible composition of claim 5, wherein the sweetener is selected from the group consisting of agave inulin, agave nectar, agave syrup, amazake, brazzein, brown rice syrup, coconut crystals, coconut sugars, coconut syrup, date sugar, fructans (also referred to as inulin fiber, fructo-oligosaccharides, or oligo-fructose), green stevia powder, stevia rebaudiana, rebaudioside A, rebaudioside B, rebaudioside C, rebaudioside D, rebaudioside E, rebaudioside F, rebaudioside I, rebaudioside H, rebaudioside L, rebaudioside K, rebaudioside J, rebaudioside N, rebaudioside O, rebaudioside M and other sweet stevia-based glycosides, stevioside, stevioside extracts, honey, Jerusalem artichoke syrup, licorice root, luo han guo (fruit, powder, or extracts), lucuma (fruit, powder, or extracts), maple sap (including, for example, sap extracted from Acer saccharum, Acer nigrum, Acer rubrum, Acer saccharinum, Acer platanoides, Acer negundo, Acer macrophyllum, Acer grandidentatum, Acer glabrum, Acer mono), maple syrup, maple sugar, walnut sap (including, for example, sap extracted from Juglans cinerea, Juglans nigra, Juglans ailatifolia, Juglans regia), birch sap (including, for example, sap extracted from Betula papyrifera, Betula alleghaniensis, Betula lenta, Betula nigra, Betula populifolia, Betula pendula), sycamore sap (such as, for example, sap extracted from Platanus occidentalis), ironwood sap (such as, for example, sap extracted from Ostrya virginiana), mascobado, molasses (such as, for example, blackstrap molasses), molasses sugar, monatin, monellin, cane sugar (also referred to as natural sugar, unrefined cane sugar, or sucrose), palm sugar, panocha, piloncillo, rapadura, raw sugar, rice syrup, sorghum, sorghum syrup, cassava syrup (also referred to as tapioca syrup), thaumatin, yacon root, malt syrup, barley malt syrup, barley malt powder, beet sugar, cane sugar, crystalline juice crystals, caramel, carbitol, carob syrup, castor sugar, hydrogenated starch hydrolates, hydrolyzed can juice, hydrolyzed starch, invert sugar, anethole, arabinogalactan, arrope, syrup, P-4000, acesulfame potassium (also referred to as acesulfame K or ace-K), alitame (also referred to as aclame), advantame, aspartame, baiyunoside, neotame, benzamide derivatives, bernadame, canderel, carrelame and other guanidine-based sweeteners, vegetable fiber, corn sugar, coupling sugars, curculin, cyclamates, cyclocarioside I, demerara, dextran, dextrin, diastatic malt, dulcin, sucrol, valzin, dulcoside A, dulcoside B, emulin, enoxolone, maltodextrin, saccharin, estragole, ethyl maltol, glucin, gluconic acid, glucono-lactone, glucosamine, glucoronic acid, glycerol, glycine, glycyphillin, glycyrrhizin, golden sugar, yellow sugar, golden syrup, granulated sugar, gynostemma, hernandulcin, isomerized liquid sugars, jallab, chicory root dietary fiber, kynurenine derivatives (including N′-formyl-kynurenine, N′-acetyl-kynurenine, 6-chloro-kynurenine), galactitol, lites se, ligicane, lycasin, lugduname, guanidine, falernum, mabinlin I, mabinlin II, maltol, maltisorb, maltodextrin, maltotriol, mannosamine, miraculin, mizuame, mogrosides (including, for example, mogroside I (including mogrosides I_{A} and I_{E}), mogroside II (including mogrosides II_{A}, II_{A1}, II_{A2}, II_{B}, and II_{E}), mogroside III (including mogroside III_{A2}, III_{E}), mogroside IV (including mogrosides IV_{A} and IV_{E}), mogroside V, and mogroside VI), 7-oxomogrosides (including, for example, 7-oxomogroside II_{E} and 7-oxomogroside V), 11-deoxymogrosides (including, for example, 11-deoxymogrosides III and V), II-oxomogrosides (including, for example, 11-oxomogrosides I_{A}, III_{E}, IV, IV_{A}, IV_{E}, V, and VI), siamenoside, siamenoside I, isomogroside, isomogroside V, mogrol, 11-oxomogrol, neomogroside, mukurozioside, nano sugar, naringin dihydrochalcone, neohesperidine dihydrochalcone, nib sugar, nigero-oligosaccharide, norbu, orgeat syrup, osladin, pekmez, pentadin, periandrin I, perillaldehyde, perillartine, petphyllum, phenylalanine, phlomisoside I, phlorodizin, phyllodulcin, polyglycitol syrups, polypodoside A, pterocaryoside A, pterocaryoside B, rebiana, refiners syrup, rub syrup, rubusoside, selligueain A, shugr, siamenoside I, siraitia grosvenorii, soybean oligosaccharide, Splenda, SRI oxime V, steviol glycoside, steviolbioside, stevioside, strogins 1, 2, and 4, sucronic acid, sucrononate, sugar, suosan, phloridzin, superaspartame, tetrasaccharide, threitol, treacle, trilobtain, tryptophan and derivatives (6-trifluoromethyl-tryptophan, 6-chloro-D-tryptophan), vanilla sugar, volemitol, birch syrup, aspartame-acesulfame, assugrin, and combinations or blends of any two or more thereof.

10. The ingestible composition of claim 9, which is in form of a food or beverage product, an animal feed product, a pharmaceutical composition, a nutritional product, a dietary supplement, or over-the-counter medication.

11. The ingestible composition of claim 10, wherein the food or beverage product is for human or animal consumption.

12. The ingestible composition of claim 11, wherein the beverage is selected from the group consisting of sparkling beverages, fruit juices, fruit-flavored juices, juice drinks, nectars, vegetable juices, vegetable-flavored juices, sports drinks, energy drinks, enhanced water drinks, enhanced water with vitamins, near water drinks, coconut waters, tea type drinks, coffees, cocoa drinks, beverages containing milk components, milk alternative beverages, beverages containing cereal extracts and smoothies.

13. The ingestible composition of claim 10, wherein the beverage product comprises:
citric acid, phosphoric acid, ascorbic acid, sodium acid sulfate, lactic acid, and/or tartaric acid;
caffeine, quinine, green tea, catechins, polyphenols, green robusta coffee extract, green coffee extract, whey protein isolate, and/or potassium chloride;
caramel color, Red #40, Yellow #5, Yellow #6, Blue #1, Red #3, purple carrot, black carrot juice, purple sweet potato, vegetable juice, fruit juice, beta carotene, turmeric curcumin, and/or titanium dioxide;
sodium benzoate, potassium benzoate, potassium sorbate, sodium metabisulfate, sorbic acid, and/or benzoic acid;
calcium disodium EDTA, alpha tocopherols, mixed tocopherols, rosemary extract, grape seed extract, resveratrol, and/or sodium hexametaphosphate;
resveratrol, Co-Q10, omega 3 fatty acids, theanine, choline chloride, fibersol, inulin, taurine, panax ginseng extract, guanana extract, ginger extract, L-phenylalanine, L-carnitine, L-tartrate, D-glucoronolactone, inositol, bioflavonoids, Echinacea, ginko biloba, yerba mate, flax seed oil, garcinia cambogia rind extract, white tea extract, ribose, milk thistle extract, grape seed extract, pyrodixine HCl, cyanoobalamin, niacinamide, biotin, calcium lactate, calcium pantothenate, calcium phosphate, calcium carbonate, chromium chloride, chromium polynicotinate, cupric sulfate, folic acid, ferric pyrophosphate, iron, magnesium lactate, magnesium carbonate, magnesium sulfate, monopotassium phosphate, monosodium phosphate, phosphorus, potassium iodide, potassium phosphate, riboflavin, sodium sulfate, sodium gluconate, sodium polyphosphate, sodium bicarbonate, thiamine mononitrate, vitamin D3, vitamin A palmitate, zinc gluconate, zinc lactate, and/or zinc sulfate;
ester gun, brominated vegetable oil, and/or sucrose acetate isobutyrate;
sodium citrate, potassium citrate, and/or salt;
ethyl alcohol, glycerine, gum Arabic, maltodextrin, modified corn starch, dextrose, natural flavor, natural flavor with other natural flavors, natural and artificial flavors, artificial flavor, silicon dioxide, magnesium carbonate, and/or tricalcium phosphate; and
pectin, xanthan gum, carboxylmethylcellulose, polysorbate 60, polysorbate 80, medium chain triglycerides, cellulose gel, cellulose gum, sodium caseinate, modified food starch, and/or carrageenan.
